(19) United States
(12) Patent Application Publication
(10) Pub. No.: US 2025/0186567 A1
(43) Pub. Date: Jun. 12, 2025

(54) MULTIEPITOPE VACCINE FOR THE TREATMENT OF ALZHEIMER'S DISEASE

(71) Applicant: OTHAIR PROTHENA LIMITED, Dublin 2 (IE)

(72) Inventors: Robin Barbour, Walnut Creek, CA (US); Gene Kinney, Boca Raton, FL (US); Wagner Zago, San Carlos, CA (US)

(21) Appl. No.: 18/020,034

(22) PCT Filed: Aug. 6, 2021
(86) PCT No.: PCT/US2021/045062

§ 371 Date: Feb. 6, 2023

Related U.S. Application Data
(60) Provisional application No. 63/062,919, filed on Aug. 7, 2020.

(51) Int. Cl. A61K 39/00; A61P 25/28; A61P 37/04; C07K 14/47

(52) U.S. Cl. CPC A61K 39/0007; A61P 25/28; A61P 37/04; C07K 14/4711; A61K 2039/545; A61K 2039/55555; A61K 2039/55566; A61K 2039/55572; A61K 2039/55577; A61K 2039/6037; A61K 2039/645; C07K 2319/40

(57) ABSTRACT

The disclosure provides peptide compositions and immunotherapy compositions comprising an amyloid-beta (Aβ, Abeta) peptide, a tau peptide, and an alpha-synuclein peptide. The disclosure also provides methods of treating or effecting prophylaxis of Alzheimer's disease or other diseases with beta-amyloid deposition in a subject, including methods of clearing deposits, inhibiting or reducing aggregation of Aβ and tau and an alpha-synuclein, blocking the uptake by neurons, clearing amyloid, and inhibiting propagation of tau seeds and an alpha-synuclein synucleinopathies in a subject having or at risk of developing Alzheimer's disease or other diseases containing tau and amyloid-beta and an alpha-synuclein accumulations. The methods include administering to such patients the compositions comprising an amyloid-beta (Aβ) peptide and a tau peptide and an alpha-synuclein peptide.

### RELATED APPLICATIONS

This application claims the benefit of U.S. Provisional Patent Application No. 63/062,919, filed Aug. 7, 2021, which is incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the ASCII text file created on Aug. 6, 2021, having the file name “20-1085-WO_Sequence-Listing_ST25.txt” and is 191 kb in size.

### FIELD

The disclosure relates to the technical fields of immunology and medicine, and in particular to the treatment of Alzheimer's disease and other diseases of protein misfolding.

### BACKGROUND

Alzheimer's disease (AD) is a progressive disease resulting in senile dementia. Broadly speaking, the disease falls into two categories: late onset, which occurs in old age (65+ years) and early onset, which develops well before the senile period, i.e., between 35 and 60 years. In both types of disease, the pathology is the same, but the abnormalities tend to be more severe and widespread in cases beginning at an earlier age. The disease is characterized by at least two types of lesions in the brain, neurofibrillary tangles and senile plaques. Neurofibrillary tangles are intracellular deposits of microtubule associated tau protein consisting of two filaments twisted about each other in pairs. Senile plaques (i.e., amyloid plaques) are areas of disorganized neuropil up to 150 μm across with extracellular amyloid deposits at the center which are visible by microscopic analysis of sections of brain tissue. The accumulation of amyloid plaques within the central nervous system is also associated with Down's syndrome and other cognitive disorders, Cerebral amyloid angiopathy (CAA), and the ocular disease Age-Related Macular Degeneration.

A principal constituent of the plaques is a peptide termed Aβ or β-amyloid peptide. Aβ peptide is a 4-kDa internal fragment of 38-43 amino acids of a larger transmembrane glycoprotein named amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The cognitive and physical symptoms associated with this type of neural deterioration characterize Alzheimer's disease.

Another protein reported to occur at increased levels in Alzheimer's patients relative to the general population is tau, the principal constituent of neurofibrillary tangles, which together with amyloid plaques are a hallmark characteristic of Alzheimer's disease. Tau tangles constitute abnormal fibrils measuring 10 nm in diameter occurring in pairs wound in a helical fashion with a regular periodicity of 80 nm. The tau within neurofibrillary tangles is abnormally phosphorylated (hyperphosphorylated) with phosphate groups attached to specific sites on the molecule. Severe involvement of neurofibrillary tangles is seen in the layer II neurons of the entorhinal cortex, the CA1 and subicular regions of the hippocampus, the amygdala, and the deeper layers (layers III, V, and superficial VI) of the neocortex in Alzheimer's disease. Tau pathologies are known to correlate to cognitive decline.

Alpha-synuclein, a protein found in neurons and other cells, is a major component of pathology that characterizes several neurodegenerative disorders including Parkinson's disease, dementia with Lewy bodies, and multiple system atrophy, which collectively are termed synucleinopathies. The understanding of the normal physiological function of alpha-synuclein is limited, but evidence indicates that soluble forms of the protein may interact with other proteins and certain intracellular membranes. In synucleinopathies, the alpha-synuclein protein appears to be abnormally aggregated intracellularly, which contributes to disease pathology. There is increasing evidence that certain aggregated forms of alpha-synuclein can be transmitted from neuron to neuron, resulting in a propagation of pathology that causes neuronal dysfunction and loss. Alpha-synuclein (SNCA) misfolding and aggregation can often be accompanied by β-amyloid deposition in some neurodegenerative diseases, and alpha-synuclein and tau aggregates coexist in several neurodegenerative disorders, including Alzheimer's disease and Parkinson's disease.

Accordingly, there exists the need for new therapies and reagents for the prevention or treatment of Alzheimer's disease, in particular, therapies and reagents capable of causing an immune response to the Aβ, Tau and alpha-synuclein present in patients.

### SUMMARY

In some embodiments, disclosure is directed to a polypeptide including a first peptide comprising 3-10 amino acids from residues 1-10 or 12-25 of SEQ ID NO:01, a second peptide including 3-13 amino acids from residues 244-400 of SEQ ID NO:02, and a third peptide comprising 3-10 amino acids from residues 81-140 of SEQ ID NO:58. In certain embodiments, the polypeptide further comprises a fourth peptide selected from one of: (a) a peptide comprising 3-10 amino acids from residues 1-10 or 12-25 of SEQ ID NO:01; (b) a peptide comprising 3-13 amino acids from residues 244-400 of SEQ ID NO:02; and (c) a peptide comprising 3-10 amino acids from residues 81-140 of SEQ ID NO:58. In some embodiments, the first peptide, second peptide, third peptide, and fourth peptide are arranged in any order in the polypeptide. For example, the second peptide may be from the microtubule binding region (MTBR) of tau (residues 244-372 of SEQ ID NO:02). In addition, the first peptide may include an amino acid sequence of one of SEQ ID NOS: 3 to 38 or 1002-1057, the second peptide may include an amino acid sequence of one of SEQ ID NOS: 39 to 57, or 142 to 1000, the third peptide may include an amino acid sequence of one of SEQ ID NOS: 59-129, and the fourth peptide, if present, is one of any one of the amino acid sequences of SEQ ID NO: 3-38, 1002-1057, 39-57, 142 to 1000, and 59-129. For example, the first polypeptide may be DAEFRHD (SEQ ID NO:06), or EFRHDSG (SEQ ID NO:19), the second polypeptide may be 5-10 amino acids, for example QIVYKPV (SEQ ID NO:39), or NIKHVPG (SEQ ID NO:57), the third polypeptide may be PDNEAYE (SEQ ID NO:75), or DPDNEAY (SEQ ID NO:69), and the fourth polypeptide, if present, may be NIKHVP (SEQ ID NO:48), or QIVYKPV (SEQ ID NO:39).

In other embodiments, two or more of the first peptide, the second peptide, and the third peptide, and the fourth peptide, if present, may be linked by a cleavable linker, which may be an amino acid sequence. A cleavable peptide linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the cleavable peptide linker is 1 amino acid, 2 amino acids, 3 amino acids, 4 amino acids, 5 amino acids, 6 amino acids, 7 amino acids, 8 amino acids, 9 amino acids, or 10 amino acids. For example, the linker may be arginine-arginine (Arg-Arg), arginine-valine-arginine-arginine (Arg-Val-Arg-Arg (SEQ ID NO:138)), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; SEQ ID NO:139), Ala-Gly-Ala-Gly (SEQ ID NO:140), or Lys-Gly-Lys-Gly (SEQ ID NO:141). In particular embodiments, the polypeptide may be DAEFRHDRRPDNEAYERRQIVYKPVKKC (SEQ ID NO:130), DAEFRHDRRQIVYKPVRRPDNEAYEKKC (SEQ ID NO:131), DAEFRHDRRPDNEAYERRNIKHVPGKKC(SEQ ID NO: 132), DAEFRHDRRNIKHVPGRRPDNEAYEKKC (SEQ ID NO:133), DAEFRHDRRQIVYKPVRRPDNEAYERRNIKHVPGGC (SEQ ID NO:134), DAEFRHDRRDPDNEAYRRNIKHVPGRRQIVYKPVGGC (SEQ ID NO:135), EFRHDSGRRQIVYKPVRRPDNEAYERRNIKHVPGGC (SEQ ID NO:136), EFRHDSGRRDPDNEAYRRNIKHVPGRRQIVYKPVGGC (SEQ ID NO:137), DAEFRHDRRDPDNEAYERRENLKHQPGGGC (SEQ ID NO:1058), DAEFRHDRRENLKHQPGRRDPDNEAYEGGC (SEQ ID NO: 1059), DAEFRHDRRPDNEAYERRENLKHQPGGGC (SEQ ID NO:1060), DAEFRHDRRENLKHQPGRRPDNEAYEGGC (SEQ ID NO:1061), DAEFRHDRRSKIGSKDNIKHRRDPDNEAYEGGC (SEQ ID NO:1062), or DAEFRHDRRDPDNEAYERRSKIGSKDNIKHGGC (SEQ ID NO:1063).

In certain embodiments, the polypeptide further comprises a blocked amine at the N-terminus.

In further embodiments, the polypeptide may include a linker to a carrier at a C-terminal portion of the polypeptide, or at a N-terminal portion of the polypeptide. A linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the linker is 1 amino acid, 2 amino acids, 3 amino acids, 4 amino acids, 5 amino acids, 6 amino acids, 7 amino acids, 8 amino acids, 9 amino acids, or 10 amino acids. For example, the linker may include an amino acid sequence of GG, GGG, AA, AAA, KK, KKK, SS, SSS, GAGA (SEQ ID NO:139), AGAG (SEQ ID NO:140), and KGKG (SEQ ID NO:141). In addition, the linker to the carrier, if present at the C-terminus, may include a C-terminal cysteine (C). For example, the polypeptide may include the amino acid sequence of DAEFRHDRRPDNEAYERRQIVYKPVKKC (SEQ ID NO:130), wherein KK and C are independently optional and, if present, KK can be replaced with GG, AA, SS, GAGA (SEQ ID NO:139), AGAG (SEQ ID NO:140), or KGKG (SEQ ID NO:141). Alternatively, the linker to the carrier, if present at the N-terminus, may include a N-terminal cysteine (C). For example, the sequence may be represented as CXX-polypeptide, wherein XX and C are independently optional and, if present, XX can be GG, AA, KK, SS, GAGA (SEQ ID NO:139), AGAG (SEQ ID NO: 140), or KGKG (SEQ ID NO:141).

In other embodiments, the disclosure is directed to an immunotherapy composition including the polypeptides of the disclosure, wherein the polypeptide may be linked to a carrier. The carrier may include serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

Still further, embodiments of the disclosure are directed to a pharmaceutical formulation includes the polypeptides or the immunotherapy compositions of the disclosure, and including at least one adjuvant. The adjuvant may be aluminum hydroxide, aluminum phosphate, aluminum sulfate, 3 De-O-acylated monophosphoryl lipid A (MPL), QS-21, QS-18, QS-17, QS-7, TQL1055, Complete Freund's Adjuvant (CFA), Incomplete Freund's Adjuvant (IFA), oil in water emulsions (such as squalene or peanut oil), CpG, polyglutamic acid, polylysine, AddaVax™, MF59®, and combinations thereof. In addition, the formulation may include a liposomal formulation, a diluent, or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Still further, embodiments of the disclosure are directed to an immunotherapy composition including a first peptide sequence comprising 3-10 amino acid residues from the first ten or 12 to 25 N-terminal residues of SEQ ID NO:01 and a second peptide sequence comprising 3-13 amino acids from residues 244-400 of SEQ ID NO:02, and a third sequence comprising 3-10 amino acids from residues 81-140 of SEQ ID NO:58. The first peptide may include an amino acid sequence of one of SEQ ID NOS: 3 to 38 or SEQ ID NOS:1002 to 1057, and the second peptide may include an amino acid sequence of one of SEQ ID NOS: 39 to 57 or 142-1000, the third peptide sequence may include an amino acid sequence of one of SEQ ID NOS:59-129, and a fourth peptide sequence, if present, is one of any one of the amino acid sequences of SEQ ID NO: 3-38, 1002-1057, 39-57, 142-1000, and 59-129. Each of the first peptide and the second peptide, the third peptide, and the fourth peptide may include a linker to a carrier at a C-terminal portion of the polypeptide, or at a N-terminal portion of the polypeptide. When present, the linker may include an amino acid sequence selected from GG, GGG, AA, AAA, KK, KKK, SS, SSS, GAGA (SEQ ID NO:139), AGAG (SEQ ID NO:140), and KGKG (SEQ ID NO:141), and may include a C-terminal cysteine (C). In some embodiments, where the C-terminal residues in the immunogen are either IVYKPV (SEQ ID NO:194), VYKPV (SEQ ID NO: 195), YKPV (SEQ ID NO:196), KPV, or PV the linker is an amino acid linker that does not have a N-terminal glycine (e.g., GG, GAGA (SEQ ID NO:139)). The carrier may include serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

In addition, the immunotherapy composition may include at least one pharmaceutically acceptable diluent and/or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

The immunotherapy composition may be included in a pharmaceutical composition including the immunotherapy composition and at least one adjuvant such as aluminum hydroxide, aluminum phosphate, aluminum sulfate, 3 De-O-acylated monophosphoryl lipid A (MPL), QS-21, QS-18, QS-17, QS-7, TQL1055, Complete Freund's Adjuvant (CFA), Incomplete Freund's Adjuvant (IFA), oil in water emulsions (such as squalene or peanut oil), CpG, polyglutamic acid, polylysine, AddaVax™, MF59®, and combinations thereof.

Embodiments of the disclosure are also directed to nucleic acid sequences encoding the polypeptides and the immunotherapy compositions of the disclosure. The nucleic acids may be included in a nucleic acid immunotherapy composition including the nucleic acid and at least one adjuvant.

Still further, embodiments of the disclosure are directed to a methods for treating or effecting prophylaxis of Alzheimer's disease in a subject, and methods for inhibiting or reducing aggregation of at least one of Aβ, tau, and alpha-synuclein in a subject having or at risk of developing Alzheimer's disease. The methods include administrating to the subject an immunotherapy composition, a nucleic acids immunotherapy composition, or a pharmaceutical formulation of the disclosure.

The methods of the disclosure may include repeating the administering at least a second time, at least a third time, at least a fourth time, at least a fifth time, or at least a sixth time, and may include repeating the administering at an interval of about 21 to about 28 days.

Still further, methods of the disclosure are directed to inducing an immune response in an animal. The methods include administering to the animal a polypeptide, an immunotherapy composition, a pharmaceutical formulation or a nucleic acid immunotherapy composition of the disclosure in a regimen effective to generate an immune response including antibodies that specifically bind to Aβ, and/or tau, and/or alpha-synuclein. The immune response may include antibodies that specifically bind to the N-terminal region of Aβ and/or the microtubule region of tau, and/or the C-terminal region of alpha-synuclein.

In other embodiments, the disclosure is directed to an immunization kit including an immunotherapy composition of the disclosure and may include an adjuvant, wherein the immunotherapy composition may be in a first container and the adjuvant may be a second container.

Still further, the disclosure is directed to a kit including a nucleic acid immunotherapy composition of the disclosure and may include an adjuvant. The nucleic acid may be in a first container and the adjuvant may be in a second container.

### BRIEF DESCRIPTION OF THE DRAWINGS

FIG. 1A shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 25 (DAEFRHDRRQIVYKPVRRPDNEAYEKKC; SEQ ID NO:131) on day 0, week 3, and week 7, and serum was collected one week after each injection (i.e., week 1, week 4 and week 8).

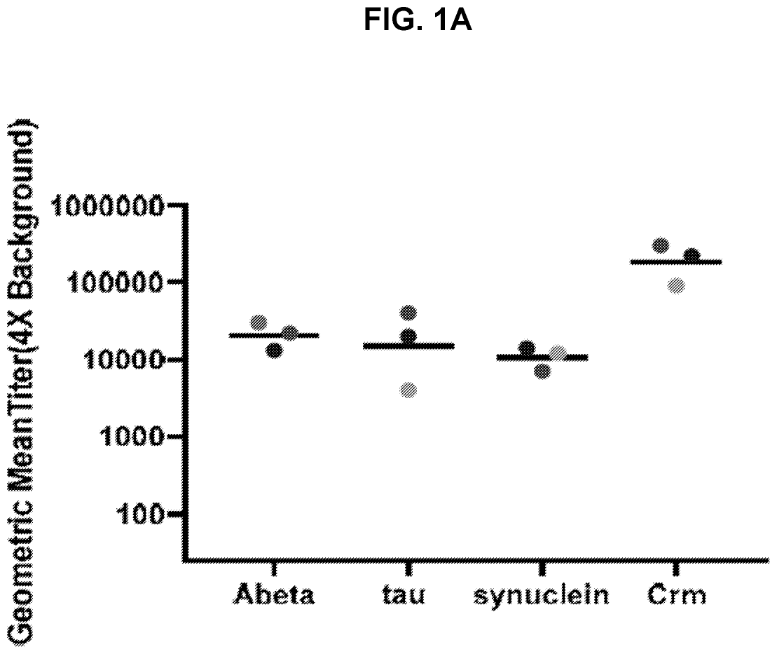

FIG. 1B shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 27 (DAEFRHDRRNIKHVPGRRPDNEAYEKKC; SEQ ID NO:133) on day 0, week 3, and week 7, and serum was collected one week after each injection (i.e., week 1, week 4 and week 8).

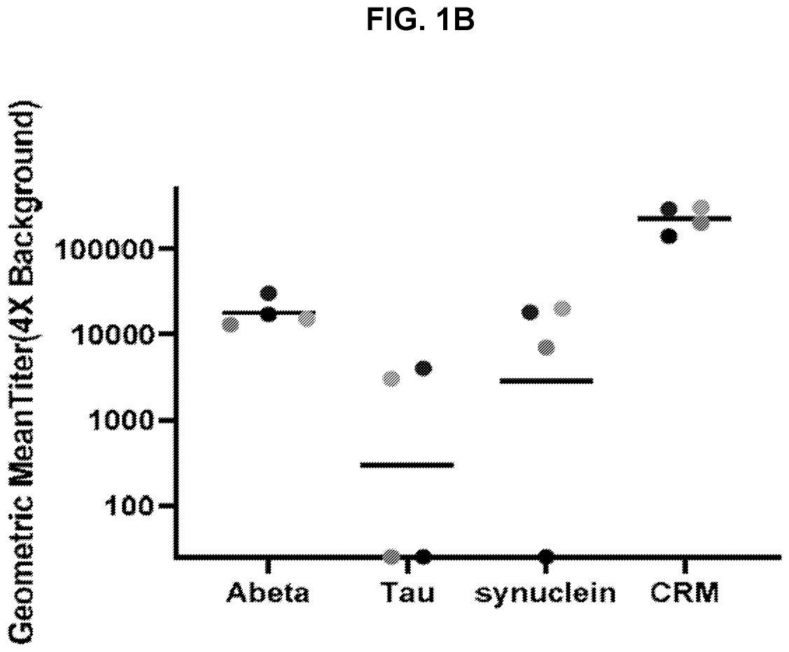

FIG. 1C shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 26 (DAEFRHDRRPDNEAYERRNIKHVPGKKC; SEQ ID NO:132) on day 0, week 3, and week 7, and serum was collected one week after each injection (i.e., week 1, week 4 and week 8).

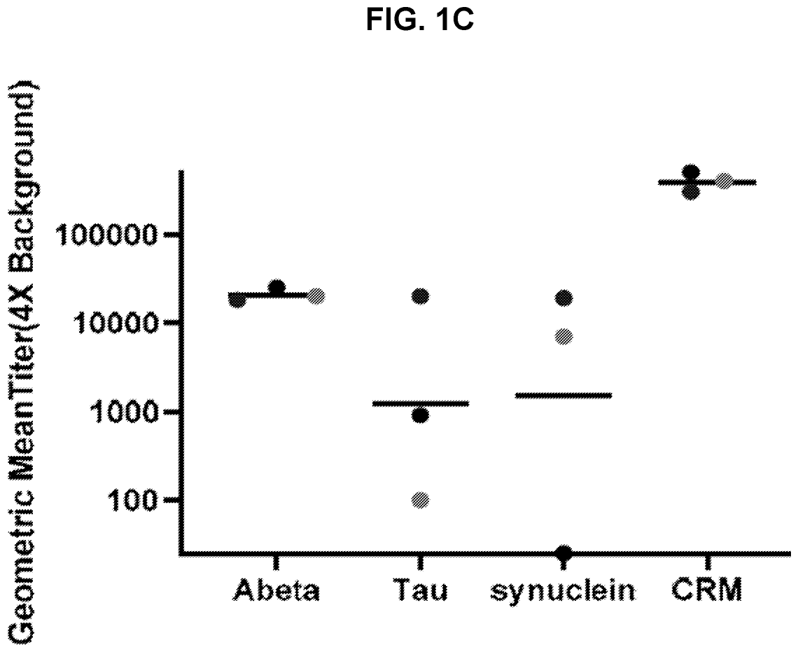

FIG. 1D shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 24 (DAEFRHDRRPDNEAYERRQIVYKPVKKC; SEQ ID NO:130) on day 0, week 3, and week 7, and serum was collected one week after each injection (i.e., week 1, week 4 and week 8).

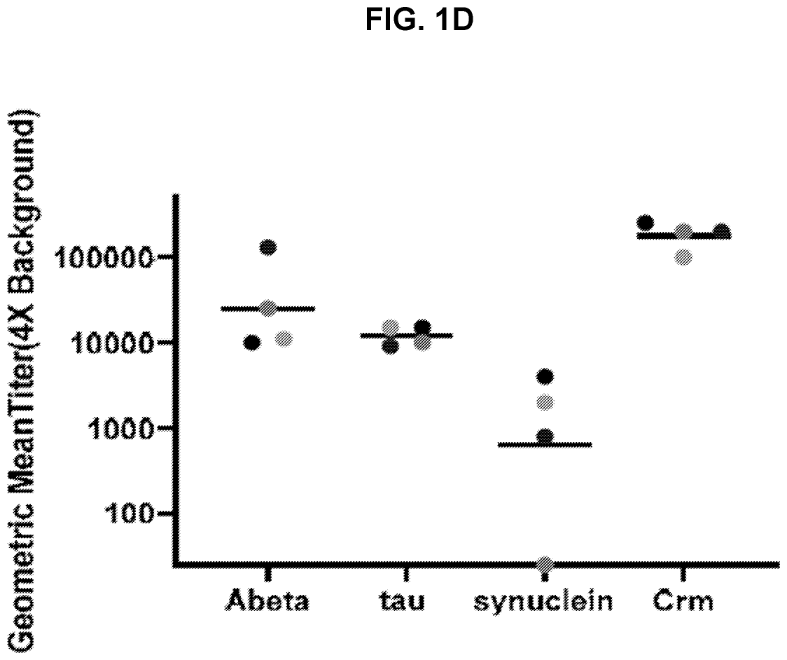

FIG. 2A shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 25 (DAEFRHDRRQIVYKPVRRPDNEAYEKKC; SEQ ID NO:131) on day 0, week 3, week 7, and week 11, and serum was collected one week after each injection (i.e., week 1, week 4, week 8, and week 12).

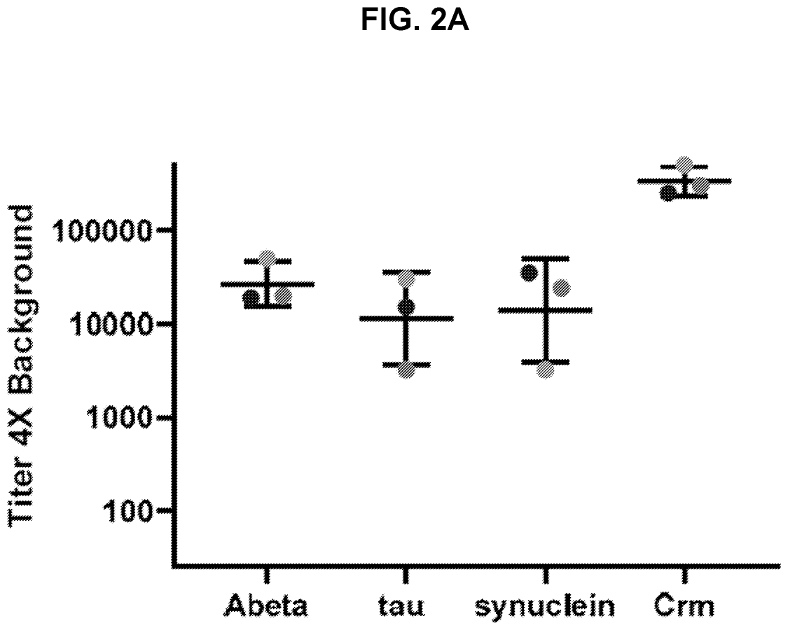

FIG. 2B shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 24 (DAEFRHDRRPDNEAYERRQIVYKPVKKC; SEQ ID NO:130) on day 0, week 3, week 7, and week 11, and serum was collected one week after each injection (i.e., week 1, week 4, week 8, and week 12).

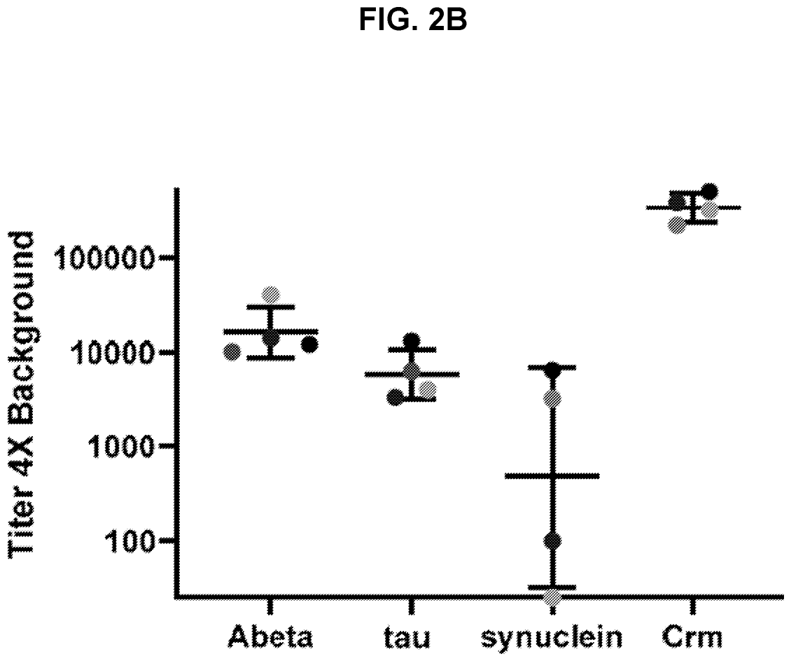

FIG. 2C shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 27 (DAEFRHDRRNIKHVPGRRPDNEAYEKKC; SEQ ID NO:133) on day 0, week 3, week 7, and week 11, and serum was collected one week after each injection (i.e., week 1, week 4, week 8, and week 12).

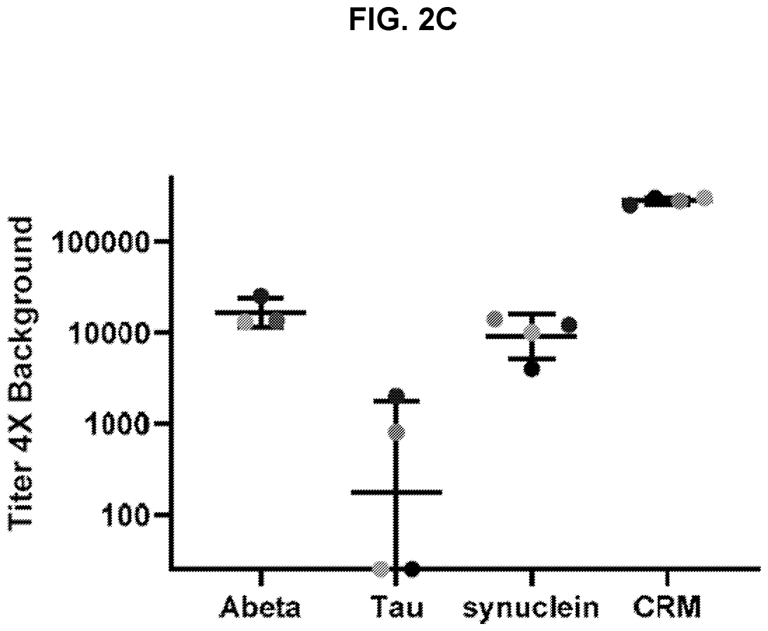

FIG. 2D shows the serum of guinea pigs vaccinated with a tri-peptide immunogen comprising an Aβ peptide, a Tau peptide and an alpha-synuclein peptide antigen produce titers to Aβ, Tau, and alpha-synuclein. Three guinea pigs were injected with Immunogen 26 (DAEFRHDRRPDNEAYERRNIKHVPGKKC; SEQ ID NO:132) on day 0, week 3, week 7, and week 11, and serum was collected one week after each injection (i.e., week 1, week 4, week 8, and week 12).

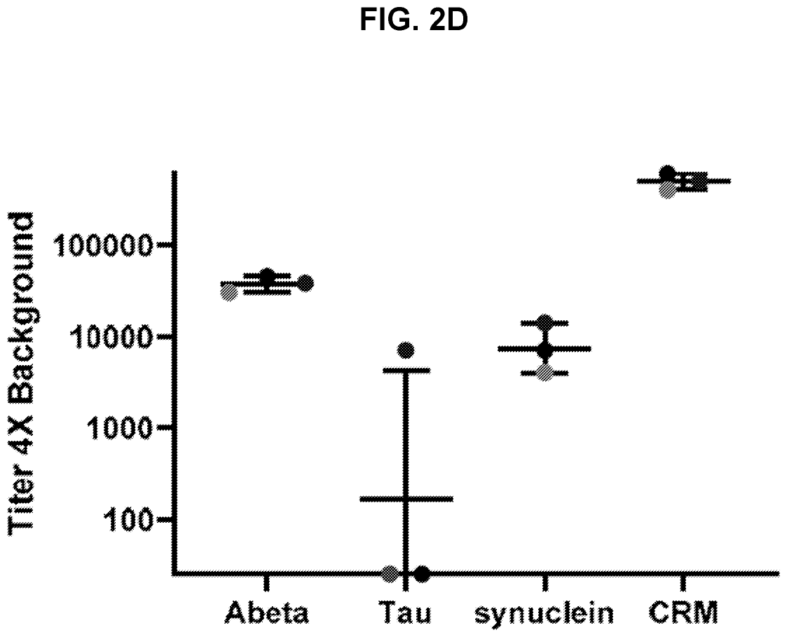

FIG. 3 shows that the serum of mice vaccinated with one of 4 tri-peptide immunogens each comprising an Aβ peptide, a Tau peptide and an alpha-synuclein peptide antigen produce titers to Aβ, Tau, and alpha-synuclein. Four Swiss webster mice per immunogen were injected on day 0 and day 10, and serum was collected on day 16. Tri 2 is DAEFRHDRRDPDNEAYERRENLKHQPGGGC (SEQ ID NO:1058), Tri 1 is DAEFRHDRRENLKHQPGRRDPDNEAYEGGC (SEQ ID NO: 1059), Tri 4 is DAEFRHDRRPDNEAYERRENLKHQPGGGC (SEQ ID NO:1060), Tri 3 is DAEFRHDRRENLKHQPGRRPDNEAYEGGC (SEQ ID NO:1061) (also see Table 4).

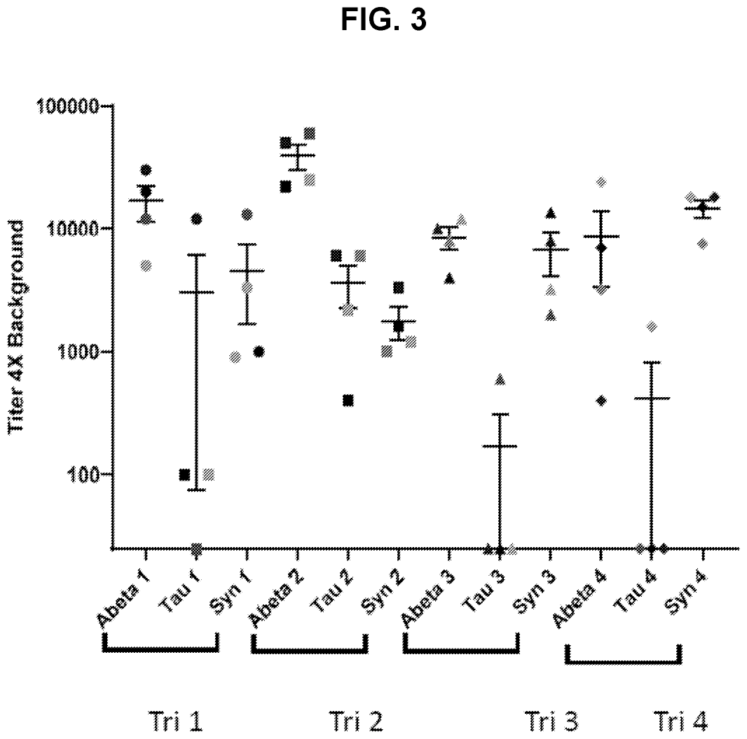

### DESCRIPTION

The disclosure provides peptide compositions and immunotherapy compositions comprising an amyloid-beta (Aβ) peptide, a tau peptide, and an alpha-synuclein peptide. The disclosure also provides methods of treating or effecting prophylaxis of Alzheimer's disease or other diseases with beta-amyloid deposition in a subject, including methods of clearing and preventing formation of deposits, inhibiting or reducing aggregation of Aβ and/or tau, and/or alpha-synuclein, blocking the binding and/or uptake of Aβ and/or tau and/or alpha-synuclein by neurons, inhibiting transmission of tau species between cells, and inhibiting propagation of pathology between brain regions in a subject having or at risk of developing Alzheimer's disease or other diseases containing tau and/or amyloid-beta accumulations. The methods include administering to such patients the compositions comprising an amyloid-beta (Aβ) peptide, a tau peptide, and an alpha-synuclein peptide.

A number of terms are defined below. As used herein, the singular forms “a,” “an”, and “the” include plural referents unless the context clearly dictates otherwise. For example, the term “a compound” or “at least one compound” can include a plurality of compounds, including mixtures thereof.

Unless otherwise apparent from the context, the term “about” encompasses insubstantial variations, such as values within a standard margin of error of measurement (e.g., SEM) of a stated value. For example the term “about” as used herein when referring to a measurable value such as a parameter, an amount, a temporal duration, can encompass variations of +/−10% or less, +/−5% or less, or +/−1% or less or less of and from the specified value. Designation of a range of values includes all integers within or defining the range, and all subranges defined by integers within the range. As used herein, statistical significance means p≤0.05.

Compositions or methods “comprising” or “including” one or more recited elements may include other elements not specifically recited. For example, a composition that “comprises” or “includes” a polypeptide sequence may contain the sequence alone or in combination with other sequences or ingredients.

An individual is at increased risk of a disease if the subject has at least one known risk-factor (e.g., age, genetic, biochemical, family history, and situational exposure) placing individuals with that risk factor at a statistically significant greater risk of developing the disease than individuals without the risk factor.

The term “patient” includes human and other mammalian subjects that receive either prophylactic or therapeutic treatment, including treatment naïve subjects. As used herein, the terms “subject” or “patient” refer to any single subject for which treatment is desired, including other mammalian subjects such as, humans, cattle, dogs, guinea pigs, rabbits, and so on. Also intended to be included as a subject are any subjects involved in clinical research trials not showing any clinical sign of disease, or subjects involved in epidemiological studies, or subjects used as controls.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” refers to objective evidence of a disease as observed by a physician.

As used herein, the terms “treat” and “treatment” refer to the alleviation or amelioration of one or more symptoms or effects associated with the disease, prevention, inhibition or delay of the onset of one or more symptoms or effects of the disease, lessening of the severity or frequency of one or more symptoms or effects of the disease, and/or increasing or trending toward desired outcomes as described herein.

The terms “prevention”, “prevent”, or “preventing” as used herein refer to contacting (for example, administering) the peptide(s) or immunotherapy compositions of the present disclosure with a subject before the onset of a disease, with or without Aβ and/or tau pathology already present (primary and secondary prevention), thereby delaying the onset of clinical symptoms and/or alleviating symptoms of the disease after the onset of the disease, compared to when the subject is not contacted with the peptide or immunotherapy compositions, and does not refer to completely suppressing the onset of the disease. In some cases, prevention may occur for limited time after administration of the peptide or immunotherapy compositions of the present disclosure. In other cases, prevention may occur for the duration of a treatment regimen comprising administering the peptide or immunotherapy compositions of the present disclosure.

The terms “reduction”, “reduce”, or “reducing” as used herein refer to decreasing the amount of Aβ and/or tau and/or alpha-synuclein present in a subject or in tissue of the subject, or suppressing an increase in the amount of Aβ and/or tau and/or alpha-synuclein present in a subject or in tissue of the subject, which encompasses decreasing or suppressing an increase in (e.g., decreasing the rate of increase) the amount of Aβ and/or tau and/or alpha-synuclein present, accumulated, aggregated, or deposited in the subject or tissue in the subject. In certain embodiments, the decrease in or suppression of an increase in (e.g., decreasing the rate of increase) the amount of Aβ and/or tau and/or alpha-synuclein present, accumulated, aggregated, or deposited in the subject refers to an amount of Aβ and/or tau and/or alpha-synuclein present, accumulated, aggregated, or deposited in the central nervous system (CNS) of the subject. In certain embodiments, the decrease in or suppression of an increase in (e.g., decreasing the rate of increase) the amount of Aβ and/or tau and/or alpha-synuclein present, accumulated, aggregated, or deposited in the subject refers to an amount of Aβ and/or tau and/or alpha-synuclein present, accumulated, aggregated, or deposited in the periphery (e.g., peripheral circulatory system) of the subject. In certain embodiments, the decrease in or suppression of an increase in (e.g., decreasing the rate of increase) the amount of Aβ and/or tau and/or alpha-synuclein present, accumulated, aggregated, or deposited in the subject refers to an amount of Aβ and/or tau and/or alpha-synuclein present, accumulated, aggregated, or deposited in the brain of the subject. In some embodiments, the Aβ and/or tau and/or alpha-synuclein reduced is the pathological form(s) of the Aβ (e.g., extracellular plaque deposits of the β-amyloid peptide (Aβ), neuritic amyloid plaques), and/or tau (e.g., neurofibrillary tangles of tau, dystrophic neurites), and/or alpha-synuclein (e.g., fibular alpha-synuclein inclusions, oligomeric or fibrillar alpha-synuclein conglomerates, and protofibrillar intermediates of alpha-synuclein oligomers). In yet other embodiments, pathological indicators of neurodegenerative disease and/or synucleinopathies are decreased.

The terms “epitope” or “antigenic determinant” refers to a site on an antigen to which B and/or T cells respond, or to a site on an antigen to which an antibody binds. Epitopes can be formed both from contiguous amino acids or from noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, at least 4, at least 5, at least 6, at least 7, at least 8, at least 9, at least 10, at least 11, at least 12, or at least 13 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996).

An “immunogenic agent” or “immunogen” or “antigen” is capable of inducing an immunological response against itself or modified/processed versions of itself upon administration to an animal, optionally in conjunction with an adjuvant. The terms “immunogenic agent” or “immunogen” or “antigen” refer to a compound or composition comprising a peptide, polypeptide or protein which is “antigenic” or “immunogenic” when administered in an appropriate amount (an “immunogenically effective amount”), i.e., capable of inducing, eliciting, augmenting or boosting a cellular and/or humoral immune response and of being recognized by the products of that response (T cells, antibodies). An immunogen can be a peptide, or a combination of two or more same or different peptides, that includes at least 3, at least 4, at least 5, at least 6, at least 7, at least 8, at least 9, at least 10, at least 11, at least 12, or at least 13 amino acids in a liner or spatial conformation.

An immunogen may be effective when given alone or in combination, or linked to, or fused to, another substance (which can be administered at one time or over several intervals). An immunogenic agent or immunogen may include an antigenic peptide or polypeptide that is linked to a carrier as described herein.

A nucleic acid such as DNA or RNA that encodes an antigenic peptide, or polypeptide is referred to as a “DNA [or RNA] immunogen,” as the encoded peptide or polypeptide is expressed in vivo after administration of the DNA or RNA. The peptide or polypeptide can be recombinantly expressed from a vaccine vector, which can be naked DNA or RNA that comprises the peptide or polypeptide coding sequence operably linked to a promoter, e.g., an expression vector or cassette as described herein.

The term “adjuvant” refers to a compound that, when administered in conjunction with an antigen, augments the immune response to the antigen, but when administered alone does not generate an immune response to the antigen. Adjuvants can augment an immune response by several mechanisms including lymphocyte recruitment, stimulation of B and/or T cells, and stimulation of macrophages. An adjuvant may be a natural compound, a modified version of or derivative of a natural compound, or a synthetic compound.

The terms “peptide” and “polypeptide” are used interchangeably herein and refer to a chain of two or more consecutive amino acids. If and when a distinction is made, context makes the meaning clear. For example, if two or more peptides described herein are joined to make a dimeric or multimeric peptide, polypeptide may be used to indicate “poly” or “more than one” peptide.

The term “pharmaceutically acceptable” means that the carrier, diluent, excipient, adjuvant, or auxiliary is compatible with the other ingredients of a pharmaceutical formulation and not substantially deleterious to the recipient thereof.

The terms “immunotherapy” or “immune response” refer to the development of a beneficial humoral (antibody mediated) and/or a cellular (mediated by antigen-specific T cells or their secretion products) response directed against an Aβ and/or tau peptide in a recipient. Such a response can be an active response induced by administration of immunogen (e.g. an Aβ peptide and/or tau peptide and/or alpha-synuclein peptide). A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

### Amyloid Beta (Aβ)

Aβ (also referred to herein as beta amyloid peptide or Abeta) peptide is about a 4-kDa internal fragment of 38-43 amino acids of APP (Aβ39, Aβ40, Aβ41, Aβ42, and Aβ43). Aβ40, for example, consists of residues 672-711 of APP and Aβ42 consists of residues 673-713 of APP. As a result of proteolytic processing of APP by different secretase enzymes in vivo or in situ, Aβ is found in both a “short form”, 40 amino acids in length, and a “long form”, ranging from 42-43 amino acids in length. Epitopes or antigenic determinants, as described herein, are located within the N-terminus of the Aβ peptide and include residues within amino acids 1-10 and 12-25 of Aβ, for example from residues 1-3, 1-4, 1-5, 1-6, 1-7, or 3-7 of Aβ42. Additional examples of epitopes or antigenic determinants include residues 2-4, 2-5, 2-6, 2-7, or 2-8 of Aβ, residues 3-5, 3-6, 3-7, 3-8, or 3-9 of Aβ, or residues 4-7, 4-8, 4-9, or 4-10 of Aβ42. Aβ residues 12-24, 12-23, 12-22, 13-25, 13-24, 13-23, 13-22, 14-25, 14-24, 14-23, 14-22, 15-25, 15-24, 15-23, or 15-22 of Aβ. For example, from residues 12-17, 12-18, 12-19, 12-20, 12-21, 13-17, 13-18, 13-19, 13-20, 13-21, 13-22, 14-17, 14-18, 14-19, 14-20, 14-21, 14-22, 14-23, 15-17, 15-18, 15-19, 15-20, 15-21, 15-22, 15-23, or 15-24 of Aβ42. Additional examples of epitopes or antigenic determinants include residues 16-18, 16-19, 16-20, 16-21, 16-22, 16-23, 16-24, 16-25, 17-19, 17-20, 17-21, 17-22, 17-23, 17-24 or 17-25 of Aβ42. Other examples of epitopes or antigenic determinants include residues 18-20, 18-21, 18-22, 18-23, 18-24, 18-25, 19-21, 19-22, 19-23, 19-24, 19-25, 20-22, 20-23, 20-24, 20-25, 21-23, 21-24 or 21-25 of Aβ42.

Aβ (Abeta) is the principal component of characteristic plaques of Alzheimer's disease. Aβ is generated by processing of a larger protein APP by two enzymes, termed beta and gamma secretases. Known mutations in APP associated with Alzheimer's disease occur proximate to the site of beta or gamma secretase, or within Aβ. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize Alzheimer's disease.

### Tau

Tau is a protein with a molecular weight of about 50,000 that is normally present in nerve axons or the like, and contributes to microtubular stability. The tau proteins (or τ proteins) are a group of six highly-soluble protein isoforms produced by alternative splicing from the gene MAPT (microtubule-associated protein tau). They have roles primarily in maintaining the stability of microtubules in axons and are abundant in the neurons of the central nervous system (CNS). They are less common elsewhere but are also expressed at very low levels in CNS astrocytes and oligodendrocytes. Pathologies and dementias of the nervous system such as Alzheimer's disease and Parkinson's disease are associated with tau proteins that have become hyperphosphorylated insoluble aggregates called neurofibrillary tangles. Pathogenic tau species causes toxic effects through direct binding to cells and/or accumulation inside cells and/or initiation of misfolding processes (seeding) and is can be propagated from one cell to another via cell-to-cell transmission. Toxicity could also happen by neurofibrillary tangles (NFTs), which leads to cell death and cognitive decline. Other tauopathies include, for example, progressive supranuclear palsy, corticobasal syndrome, some frontotemporal dementias, and chronic traumatic encephalopathy.

### Alpha-Synuclein

Alpha-synuclein is a highly conserved protein that is abundant in neurons, especially presynaptic terminals. Aggregated alpha-synuclein proteins form brain lesions that are hallmarks of neurodegenerative synucleinopathies. Furthermore, misfolding and aggregation can often be accompanied by β-amyloid deposition in some neurodegenerative diseases, and alpha-synuclein and tau aggregates coexist in several neurodegenerative disorders, including Alzheimer's disease and Parkinson's disease.

### Aβ/Tau/Alpha-Synuclein Polypeptides of an Immunogen

An agent used for active immunization can induce in a patient an immune response and can serve as an immunotherapy. Agents used for active immunization can be, for example, the same types of immunogens used for generating monoclonal antibodies in laboratory animals, and may include 3, 4, 5, 6, 7, 8, 9, 10, 11, 12, or 13 or more contiguous amino acids from a region of Aβ and/or tau peptide and/or alpha-synuclein peptide. In each of the embodiments of the peptides described herein, the peptides may comprise, consist, or consist essentially of the recited sequences.

In some embodiments of the disclosure, an Aβ/tau/alpha-synuclein immunogen can include an Aβ peptide comprising 3-10 amino acids from residues 1-10 or 12-25 of the N-terminal sequence of Aβ (SEQ ID NO:01) linked to a tau peptide comprising 3-13 amino acids from residues 244-400 of the long form of tau (SEQ ID NO:02), and an alpha-synuclein peptide comprising 3-10 amino acids from residues 81-140 of SEQ ID NO:58. For example, the tau peptide may comprise 3-13 amino acids from the microtubule binding region of tau (residues 344-372 of SEQ ID NO:02).

In some embodiments of the disclosure, the Aβ peptide can include 3-10 amino acids from residues 1-10 or 12-25 of DAEFRHDSGYEVHHQKLVFFAEDVGSNKGAIIGLMVGGVVIA (SEQ ID NO:01). For example, the Aβ peptide is selected from the following:

(SEQ ID NO: 03)
DAEFRHDSGY
(SEQ ID NO: 04)
DAEFRHDSG,
(SEQ ID NO: 05)
DAEFRHDS,
(SEQ ID NO: 06)
DAEFRHD,
(SEQ ID NO: 07)
DAEFRH,
(SEQ ID NO: 08)
DAEFR,
(SEQ ID NO: 09)
DAEF,
(SEQ ID NO: 10)
DAE,
(SEQ ID NO: 11)
AEFRHDSGY,
(SEQ ID NO: 12)
AEFRHDSG,
(SEQ ID NO: 13)
AEFRHDS,
(SEQ ID NO: 14)
AEFRHD,
(SEQ ID NO: 15)
AEFRH,
(SEQ ID NO: 16)
AEFR,
(SEQ ID NO: 17)
AEF,
(SEQ ID NO: 18)
EFRHDSGY,
(SEQ ID NO: 19)
EFRHDSG,
(SEQ ID NO: 20)
EFRHDS,
(SEQ ID NO: 21)
EFRHD,
(SEQ ID NO: 22)
EFRH,
(SEQ ID NO: 23)
EFR,
(SEQ ID NO: 24)
FRHDSGY,
(SEQ ID NO: 25)
FRHDSG,
(SEQ ID NO: 26)
FRHDS,
(SEQ ID NO: 27)
FRHD,
(SEQ ID NO: 28)
FRH,
(SEQ ID NO: 29)
RHDSGY,
(SEQ ID NO: 30)
RHDSG,
(SEQ ID NO: 31)
RHDS,
(SEQ ID NO: 32)
RHD,
(SEQ ID NO: 33)
HDSGY,
(SEQ ID NO: 34)
HDSG,
(SEQ ID NO: 35)
HDS,
(SEQ ID NO: 36)
DSGY,
(SEQ ID NO: 37)
DSG,
(SEQ ID NO: 38)
SGY,
(SEQ ID NO: 1002)
VHHQKLVFFA,
(SEQ ID NO: 1003)
VHHQKLVFF,
(SEQ ID NO: 1004)
VHHQKLVF,
(SEQ ID NO: 1005)
VHHQKLV,
(SEQ ID NO: 1006)
VHHQKL,
(SEQ ID NO: 1007)
HHQKLVFFAE,
(SEQ ID NO: 1008)
HHQKLVFFA,
(SEQ ID NO: 1009)
HHQKLVFF,
(SEQ ID NO: 1010)
HHQKLVF,
(SEQ ID NO: 1011)
HHQKLV,
(SEQ ID NO: 1012)
HHQKL,
(SEQ ID NO: 1013)
HQKLVFFAED,
(SEQ ID NO: 1014)
HQKLVFFAE,
(SEQ ID NO: 1015)
HQKLVFFA,
(SEQ ID NO: 1016)
HQKLVFF,
(SEQ ID NO: 1017)
HQKLVF,
(SEQ ID NO: 1018)
HQKLV,
(SEQ ID NO: 1019)
HQKL,
(SEQ ID NO: 1020)
QKLVFFAEDV,
(SEQ ID NO: 1021)
QKLVFFAED,
(SEQ ID NO: 1022)
QKLVFFAE,
(SEQ ID NO: 1023)
QKLVFFA,
(SEQ ID NO: 1024)
QKLVFF,
(SEQ ID NO: 1025)
QKLVF,
(SEQ ID NO: 1026)
QKLV,
(SEQ ID NO: 1027)
QKL,
(SEQ ID NO: 1028)
KLVFFAEDVG,
(SEQ ID NO: 1029)
KLVFFAEDV,
(SEQ ID NO: 1030)
KLVFFAED,
(SEQ ID NO: 1031)
KLVFFAE,
(SEQ ID NO: 1032)
KLVFFA,
(SEQ ID NO: 1033)
KLVFF,
(SEQ ID NO: 1034)
KLVF,
(SEQ ID NO: 1035)
KLV,
(SEQ ID NO: 1036)
LVFFAEDVG,
(SEQ ID NO: 1037)
LVFFAEDV,
(SEQ ID NO: 1038)
LVFFAED,
(SEQ ID NO: 1039)
LVFFAE,
(SEQ ID NO: 1040)
LVFFA,
(SEQ ID NO: 1041)
LVFF,
(SEQ ID NO: 1042)
LVF,
(SEQ ID NO: 1043)
VFFAEDVG,
(SEQ ID NO: 1044)
VFFAEDV,
(SEQ ID NO: 1045)
VFFAED,
(SEQ ID NO: 1046)
VFFAE,
(SEQ ID NO: 1047)
VFFA,
(SEQ ID NO: 1048)
VFF,
(SEQ ID NO: 1049)
FFAEDVG,
(SEQ ID NO: 1050)
FFAEDV,
(SEQ ID NO: 1051)
FFAED,
(SEQ ID NO: 1052)
FFAE,
(SEQ ID NO: 1053)
FFA,
(SEQ ID NO: 1054)
FAEDVG,
(SEQ ID NO: 1055)
FAEDV,
(SEQ ID NO: 1056)
FAED,
and
(SEQ ID NO: 1057)
FAE.

In certain embodiments, the Aβ peptide is DAEFRHD (SEQ ID NO:06), DAEFR (SEQ ID NO:08) or EFRHD (SEQ ID NO:21).

The tau peptide can correspond to a peptide comprising 3-13 amino acids from residues 244-400 of SEQ ID NO:02. In some embodiments, the fragment is unphosphorylated. In some embodiments, the fragment is phosphorylated. In some embodiments, the tau peptide comprises an amino acid sequence represented by the consensus motif (Q/E)IVYK(S/P) (SEQ ID NO:996). In some embodiments, the tau peptide comprises an amino acid sequence represented by the consensus motif KXXSXXNX(K/H)H (SEQ ID NO: 995) where X is any amino acid. In some embodiments, the tau peptide is selected from SEQ ID NOS: 146-1000. In some embodiments, the tau peptide is selected from the following:

(SEQ ID NO: 39)
QIVYKPV,
(SEQ ID NO: 40)
QIVYKP,
(SEQ ID NO: 41)
QIVYKSV,
(SEQ ID NO: 42)
EIVYKSV,
(SEQ ID NO: 997)
QIVYKS,
(SEQ ID NO: 43)
EIVYKSP,
(SEQ ID NO: 998)
EIVYKS,
(SEQ ID NO: 44)
EIVYKPV,
(SEQ ID NO: 999)
EIVYKP,
(SEQ ID NO: 45)
IVYKSPV,
(SEQ ID NO: 46)
IVYK,
(SEQ ID NO: 1000)
CNIKHVPG,
(SEQ ID NO: 47)
CNIKHVP,
(SEQ ID NO: 48)
NIKHVP,
(SEQ ID NO: 49)
HVPGGG,
(SEQ ID NO: 50)
HVPGG,
(SEQ ID NO: 51)
HKPGGG,
(SEQ ID NO: 52)
HKPGG,
(SEQ ID NO: 53)
KHVPGGG,
(SEQ ID NO: 54)
KHVPGG,
(SEQ ID NO: 55)
HQPGGG,
(SEQ ID NO: 56)
HQPGG,
(SEQ ID NO: 57)
NIKHVPG,
and
SEQ ID NOS: 146-996.

The alpha-synuclein peptide can correspond to a peptide comprising 3-10 amino acids from residues 81-140 of SEQ ID NO:58. In some embodiments, alpha-synuclein is unphosphorylated. In some embodiments, alpha-synuclein is phosphorylated. In some compositions, the alpha-synuclein peptide is selected from the following:

(SEQ ID NO: 59)
VDPDNEAYEM,
(SEQ ID NO: 60)
VDPDNEAYE,
(SEQ ID NO: 61)
VDPDNEAY,
(SEQ ID NO: 62)
VDPDNEA,
(SEQ ID NO: 63)
VDPDNE,
(SEQ ID NO: 64)
VDPDN,
(SEQ ID NO: 65)
VDPD,
(SEQ ID NO: 66)
VDP,
(SEQ ID NO: 67)
DPDNEAYEM,
(SEQ ID NO: 68)
DPDNEAYE,
(SEQ ID NO: 69)
DPDNEAY,
(SEQ ID NO: 70)
DPDNEA,
(SEQ ID NO: 71)
DPDNE,
(SEQ ID NO: 72)
DPDN,
(SEQ ID NO: 73)
DPD,
(SEQ ID NO: 74)
PDNEAYEM,
(SEQ ID NO: 75)
PDNEAYE,
(SEQ ID NO: 76)
PDNEAY,
(SEQ ID NO: 77)
PDNEA,
(SEQ ID NO: 78)
PDNE,
(SEQ ID NO: 79)
PDN,
(SEQ ID NO: 80)
DNEAYEM,
(SEQ ID NO: 81)
DNEAYE,
(SEQ ID NO: 82)
DNEAY,
(SEQ ID NO: 83)
DNEA,
(SEQ ID NO: 84)
DNE,
(SEQ ID NO: 85)
NEAYEM,
(SEQ ID NO: 86)
NEAYE,
(SEQ ID NO: 87)
NEAY,
(SEQ ID NO: 88)
NEA,
(SEQ ID NO: 89)
EAYEM,
(SEQ ID NO: 90)
EAYE,
(SEQ ID NO: 91)
EAY,
(SEQ ID NO: 92)
AYEM,
(SEQ ID NO: 93)
AYE,
(SEQ ID NO: 94)
YEM,
(SEQ ID NO: 95)
ATGFVKKDQL,
(SEQ ID NO: 96)
ATGFVKKDQ,
(SEQ ID NO: 97)
ATGFVKKD,
(SEQ ID NO: 98)
ATGFVKK,
(SEQ ID NO: 99)
ATGFVK,
(SEQ ID NO: 100)
ATGFV,
(SEQ ID NO: 101)
ATGF,
(SEQ ID NO: 102)
ATG,
(SEQ ID NO: 103)
TGFVKKDQL,
(SEQ ID NO: 104)
TGFVKKDQ,
(SEQ ID NO: 105)
TGFVKKD,
(SEQ ID NO: 106)
TGFVKK,
(SEQ ID NO: 107)
TGFVK,
(SEQ ID NO: 108)
TGFV,
(SEQ ID NO: 109)
TGF,
(SEQ ID NO: 110)
GFVKKDQL,
(SEQ ID NO: 111)
GFVKKDQ,
(SEQ ID NO: 112)
GFVKKD,
(SEQ ID NO: 113)
GFVKK,
(SEQ ID NO: 114)
GFVK,
(SEQ ID NO: 115)
GFV,
(SEQ ID NO: 116)
FVKKDQL,
(SEQ ID NO: 117)
FVKKDQ,
(SEQ ID NO: 118)
FVKKD,
(SEQ ID NO: 119)
FVKK,
(SEQ ID NO: 120)
FVK,
(SEQ ID NO: 121)
VKKDQL,
(SEQ ID NO: 122)
VKKDQ,
(SEQ ID NO: 123)
VKKD,
(SEQ ID NO: 124)
VKK,
(SEQ ID NO: 125)
KKDQL,
(SEQ ID NO: 126)
KKDQ,
(SEQ ID NO: 127)
KKD,
(SEQ ID NO: 128)
KDQL,
and
(SEQ ID NO: 129)
KDQ.

In each of these embodiments, the peptide may comprise, consist, or consist essentially of the recited sequences.

In some embodiments, the Aβ and/or tau and/or alpha-synuclein peptides are linked to form a multiple Aβ/tau/alpha-synuclein polypeptide. The Aβ, tau, and alpha-synuclein peptides can be linked by an intra-peptide linker. For example, a polypeptide linker located between the C-terminal of the first peptide and the N terminal of the second peptide. With or without the intra-peptide linker, the Aβ peptide and/or the tau peptide and/or the alpha-synuclein peptide may be positioned in a multiple Aβ/tau/alpha-synuclein polypeptide in any order. For example, the Aβ peptide may be positioned at the N-terminal portion of the multiple polypeptide and the alpha-synuclein peptide may be positioned at the C-terminal portion of the multiple polypeptide. Or, the tau peptide may be positioned at the N-terminal portion of the multiple polypeptide and the Aβ peptide may be positioned at the C-terminal portion of the multiple polypeptide side of the tau peptide. Reference to a first peptide or a second peptide or a third peptide or a fourth peptide herein is not intended to suggest an order of the Aβ and/or tau and/or alpha-synuclein peptides in the polypeptide of the immunogens.

In addition, the C-terminal portion of the Aβ peptide, the tau peptide, the alpha-synuclein, or the multiple Aβ/tau/alpha-synuclein polypeptide can include a linker for conjugating the peptides or the polypeptide to a carrier. Linkers that couple the peptides or multiple polypeptide to the carrier may include, for example, GG, GGG, KK, KKK, AA, AAA, SS, SSS, GAGA (SEQ ID NO:139), AGAG (SEQ ID NO:140), KGKG (SEQ ID NO:141), and the like between the peptides or dual polypeptide and the carrier and may further include a C-terminal or a N-terminal cysteine to provide a short peptide linker (e.g., G-G-C-, K-K-C-, A-A-C-, or S-S-C-). In some embodiments, where the C-terminal residues in the immunogen are either IVYKPV (SEQ ID NO:194), VYKPV (SEQ ID NO:195), YKPV (SEQ ID NO:196), KPV, or PV the linker is an amino acid linker that does not have a N-terminal glycine (e.g., GG, GAGA (SEQ ID NO:139), and KGKG (SEQ ID NO:141). In some embodiments, the linker comprises an amino acid sequence any one of AA, AAA, KK, KKK, SS, SSS, AGAG (SEQ ID NO:140), GG, GGG, GAGA (SEQ ID NO:139), and KGKG (SEQ ID NO:141). In some embodiments, any of the Aβ peptide, the tau peptide, the alpha-synuclein peptide and the multiple Aβ/tau/alpha-synuclein polypeptide may include a C-terminal cysteine without the spacer. In some embodiments, any of the Aβ peptide, the tau peptide, the alpha-synuclein peptide and the multiple Aβ/tau/alpha-synuclein polypeptide may include a N-terminal cysteine without the spacer.

When the Aβ, tau, and/or alpha-synuclein polypeptides are linked to form a multiple Aβ/tau/alpha-synuclein polypeptide, the linker may be a cleavable linker. As used herein, the term “cleavable linker” refers to any linker between the antigenic peptides that promotes or otherwise renders the Aβ peptide, the tau peptide, and/or the alpha-synuclein peptide more susceptible to separation from each other by cleavage (for example, by endopeptidases, proteases, low pH or any other means that may occur within or around the antigen-presenting cell) and, thereby, processing by the antigen-presenting cell, than equivalent peptides lacking such a cleavable linker. In some compositions, the cleavable linker is a protease-sensitive dipeptide or oligopeptide cleavable linker. In certain embodiments, the cleavable linker is sensitive to cleavage by a protease of the trypsin family of proteases. In some compositions, the cleavable linker comprises an amino acid sequence selected from the group consisting of arginine-arginine (Arg-Arg), arginine-valine-arginine-arginine (Arg-Val-Arg-Arg; SEQ ID NO:138), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; GAGA (SEQ ID NO:139)), Ala-Gly-Ala-Gly; AGAG (SEQ ID NO:140), and Lys-Gly-Lys-Gly; KGKG (SEQ ID NO:141). In some compositions, the cleavable linker is arginine-arginine (Arg-Arg).

In some embodiments of the disclosure, the multiple Aβ/tau/alpha-synuclein polypeptide comprises, consists or consists essentially of an amino acid sequence selected from SEQ ID NOs: 130-137 and SEQ ID NOs: 1058-1063.

In some embodiments, the multiple Aβ/tau/alpha-synuclein polypeptide is as follows:

[P1]-[CL1]-[P2]-[CL2]-[P3]-[L1]-[Cys]; Formula I:

[P1]-[CL1]-[P2]-[CL2]-[P3]-[CL3]-[P4]-[L1]-[Cys]; Formula II:

wherein, each of P1, P2, P3 and P4 may be independently selected from an Aβ peptide, a tau peptide and an alpha-synuclein peptide, provided that each of an Aβ peptide, a tau peptide and an alpha-synuclein peptide are selected. In embodiments, the P1 is an Aβ peptide, for example if the first peptide [P1] is an Aβ peptide then the second peptide [P2] is a tau peptide, and the third peptide [P3] is an alpha-synuclein peptide, or if [P1] is an Aβ peptide then [P2] is a tau Aβ peptide, and the fourth peptide [P4] is a tau peptide, or if [P1] is an Aβ peptide then [P2] is an alpha-synuclein peptide, and [P3] is a tau peptide, or if [P1] is an Aβ peptide then [P2] is an alpha-synuclein peptide, [P3] is a tau peptide, and [P4] is a tau peptide, and each of [CL1], [CL2], and [CL3] are cleavable linkers and [L1] is a linker, and [CL1], [CL2], [CL3], [L1], and [Cys] are optional.

Examples of the Aβ peptide include any one SEQ ID NOS 3-38 or 1002-1057.

Examples of the tau peptide include any one of SEQ ID NOS: 39-57, or 142-1000.

Examples of the alpha-synuclein peptide include any one of SEQ ID NOS: 59-129.

[CL1], [CL2], and [CL3] are optional, and when present, may be a cleavable linker. A cleavable linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the cleavable linker is 1 amino acid, 2 amino acids, 3 amino acids, 4 amino acids, 5 amino acids, 6 amino acids, 7 amino acids, 8 amino acids, 9 amino acids, or 10 amino acids. In some embodiments, the linker may be a cleavable linker having an amino acid sequence selected from the group consisting of arginine-arginine (Arg-Arg), arginine-valine-arginine-arginine (Arg-Val-Arg-Arg; SEQ ID NO:138), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; SEQ ID NO: 139), alanine-glycine-alanine-glycine (Ala-Gly-Ala-Gly; SEQ ID NO:140), and lysine-glycine-lysine-glycine (Lys-Gly-Lys-Gly; SEQ ID NO:141)).

[L1] is optional, and when present is a linker that couples the polypeptide to a carrier. A linker, if present, can be 1-10 amino acids in length. In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids, or one (1) amino acid. In some embodiments, the linker is 1 amino acid, 2 amino acids, 3 amino acids, 4 amino acids, 5 amino acids, 6 amino acids, 7 amino acids, 8 amino acids, 9 amino acids, or 10 amino acids. In some embodiments, the amino acid composition of a linker can mimic the composition of linkers found in natural multidomain proteins, where certain amino acids are overrepresented, underrepresented or equi-represented in natural linkers as compared to their abundance in whole protein. For example, threonine (Thr), serine (Ser), proline (Pro), glycine (Gly), aspartic acid (Asp), lysine (Lys), glutamine (Gln), asparagine (Asn), arginine (Arg), phenylalanine (Phe), glutamic acid (Glu) and alanine (Ala) are overrepresented in natural linkers. In contrast, isoleucine (Ile), tyrosine (Tyr), tryptophan (Trp), and cysteine (Cys) are underrepresented. In general, overrepresented amino acids were polar uncharged or charged residues, which constitute approximately 50% of naturally encoded amino acids, and Pro, Thr, and Gln were the most preferable amino acids for natural linkers. In some embodiments, the amino acid composition of a linker can mimic the composition of linkers commonly found in recombinant proteins, which can generally by classified as flexible or rigid linkers. For example, flexible linkers found in recombinant proteins are generally composed of small, non-polar (e.g. Gly) or polar (e.g. Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). An example of a widely used flexible linker is (Gly-Gly-Ser)n, (Gly-Gly-Gly-Ser)n (SEQ ID NO:1064) or (Gly-Gly-Gly-Gly-Ser)n (SEQ ID NO: 1065), where n=1-3. Adjusting the copy number “n” can optimize a linker to achieve sufficient separation of the functional immunogen domains to, e.g., maximize an immunogenic response. Many other flexible linkers have been designed for recombinant fusion proteins that can be used herein. In some embodiments, linkers can be rich in small or polar amino acids such as Gly and Ser but also contain additional amino acids such as Thr and Ala to maintain flexibility, as well as polar amino acids such as Lys and Glu to improve solubility. See, e.g., Chen, X. et al., “Fusion Protein Linkers: Property, Design and Functionality” Adv Drug Deliv Rev., 15; 65(10): 1357-1369 (203). In certain embodiments, when present, the linker can be an amino acid sequence selected from the group consisting of as GG, GGG, KK, KKK, AA, AAA, SS, SSS, G-A-G-A (SEQ ID NO: 139), A-G-A-G (SEQ ID NO:140), and K-G-K-G (SEQ ID NO: 142).

[Cys] is optional and can be helpful to conjugate the polypeptide to a carrier. When present, the Cys can be at the C-terminal portion of the polypeptide, or at the N-terminal portion of the polypeptide.

Examples of multiple Aβ/tau/alpha-synuclein polypeptide of the disclosure include the following:

TABLE 1
Formula I: [P1]-[CL1]-[P2]-[CL2]-[P3]-[L1]-[Cys]

| Immunogen | | | | | | | |
|---|---|---|---|---|---|---|---|
| Lab ID | [P1] | | [P2] | | [P3] | | |
| (SEQ | (SEQ | | (SEQ | | (SEQ | | |
| ID NO) | ID NO) | [CL1] | ID NO) | [CL2] | ID NO) | [L1] | Cys |
| 24 | DAEFRHD | RR | PDNEAYE | RR | QIVYKPV | KK | C |
| (130) | (06) | | (75) | | (39) | | |
| 25 | DAEFRHD | RR | QIVYKPV | RR | PDNEAYE | KK | C |
| (131) | (06) | | (39) | | (75) | | |
| 26 | DAEFRHD | RR | PDNEAYE | RR | NIKHVPG | KK | C |
| (132) | (06) | | (75) | | (57) | | |
| 27 | DAEFRHD | RR | NIKHVPG | RR | PDNEAYE | KK | C |
| (133) | (06) | | (57) | | (75) | | |
| Tri 2 | DAEFRHD | RR | DPDNEAYE | RR | ENLKHQPG | GG | C |
| (1058) | (06) | | (68) | | (777) | | |
| Tri 1 | DAEFRHD | RR | ENLKHQPG | RR | DPDNEAYE | GG | C |
| (1059) | (06) | | (777) | | (68) | | |
| Tri 4 | DAEFRHD | RR | PDNEAYE | RR | ENLKHQPG | GG | C |
| (1060) | (06) | | (75) | | (777) | | |
| Tri 3 | DAEFRHD | RR | ENLKHQPG | RR | PDNEAYE | GG | C |
| (1061) | (06) | | (777) | | (75) | | |
| Tri 5 | DAEFRHD | RR | SKIGSKDNIKH | RR | DPDNEAYE | GG | C |
| (1062) | (06) | | (986) | | (68) | | |
| Tri 6 | DAEFRHD | RR | DPDNEAYE | RR | SKIGSKDNIKH | GG | C |
| (1063) | (06) | | (68) | | (986) | | |

TABLE 2
Formula II: [P1]-[CL1]-[P2]-
[CL2]-[P3]-[CL3]-[P4]-[L1]-[Cys]

| Immunogen | | | | | | | | | |
|---|---|---|---|---|---|---|---|---|---|
| Lab ID | [P1] | | [P2] | | [P3] | | [P4] | | |
| (SEQ | (SEQ | | (SEQ | | (SEQ | | (SEQ | | |
| ID NO) | ID NO) | [CL1] | ID NO) | [CL2] | ID NO) | [CL3] | ID NO) | [L1] | Cys |
| 14 | DAEFRHD | RR | QIVYKPV | RR | PDNEAYE | RR | NIKHVP | GG | C |
| (134) | (06) | | (39) | | (75) | | (48) | | |
| 15 | DAEFRHD | RR | DPDNEAY | RR | NIKHVPG | RR | QIVYKPV | GG | C |
| (135) | (06) | | (69) | | (57) | | (39) | | |
| 16 | EFRHDSG | RR | QIVYKPV | RR | PDNEAYE | RR | NIKHVP | GG | C |
| (136) | (19) | | (39) | | (75) | | (48) | | |
| 17 | EFRHDSG | RR | DPDNEAY | RR | NIKHVPG | RR | QIVYKPV | GG | C |
| (137) | (19) | | (69) | | (57) | | (39) | | |

### Polypeptide Immunogens

The Aβ peptide, the tau peptide, the alpha-synuclein, and the multiple Aβ/tau/alpha-synuclein polypeptide are immunogens in accordance with the disclosure. In some embodiments, the peptides and the multiple Aβ/tau/alpha-synuclein polypeptide can be linked to a suitable carrier to help elicit an immune response. Accordingly, one or more the peptides and multiple Aβ/tau/alpha-synuclein polypeptides of the disclosure can be linked to a carrier. For example, each of the Aβ peptide, tau peptide, the alpha-synuclein peptide, and the Aβ/tau/alpha-synuclein polypeptide may be linked to the carrier with or without spacer amino acids (e.g., Gly-Gly, Gly-Gly-Gly, Ala-Ala, Ala-Ala-Ala, Lys-Lys, Lys-Lys-Lys, Ser-Ser, Ser-Ser-Ser, Gly-Ala-Gly-Ala (SEQ ID NO:139), Ala-Gly-Ala-Gly (SEQ ID NO:140), and Lys-Gly-Lys-Gly (SEQ ID NO:141)). In certain embodiments, the multiple Aβ/tau/alpha-synuclein polypeptide can be linked to a suitable carrier using a C-terminal cysteine to provide a linker between the peptide(s) and the carrier or the multiple Aβ/tau/alpha-synuclein polypeptide and the carrier. In certain embodiments, the multiple Aβ/tau/alpha-synuclein polypeptide can be linked to a suitable carrier using an N-terminal cysteine to provide a linker between the peptide(s) and the carrier. In some embodiments, where the C-terminal residues in the immunogen are IVYKPV (SEQ ID NO:194), VYKPV (SEQ ID NO:195), YKPV (SEQ ID NO:196), KPV, or PV the linker is an amino acid linker that does not have a N-terminal glycine (e.g., GG, GAGA (SEQ ID NO:139)).

Suitable carriers include, but are not limited to serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking peptide immunogens of the invention to an immunostimulatory polymer molecule (e.g., tripalmitoyl-S-glycerine cysteine (Pam3Cys), mannan (a mannose polymer), or glucan (a β 1-2 polymer)), cytokines (e.g., IL-1, IL-1 alpha and β peptides, IL-2, γ-INF, IL-10, GM-CSF), and chemokines (e.g., MIP1-α and β, and RANTES). Additional carriers include virus-like particles. In some compositions, immunogenic peptides can also be linked to carriers by chemical crosslinking. Techniques for linking an immunogen to a carrier include the formation of disulfide linkages using N-succinimidyl 3-(2-pyridylthio)propionate (SPDP), and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the epsilon-amino on a lysine, or other free amino group in other amino acids. In some embodiments, chemical crosslinking can comprise use of SBAP (succinimidyl 3-(bromoacetamido)propionate), which is a short (6.2 angstrom) cross-linker for amine-to-sulfhydryl conjugation via N-hydroxysuccinimide (NHS) ester and bromoacetyl reactive groups. A variety of such disulfide/amide-forming agents are described by Jansen et al., “Immunotoxins: Hybrid Molecules Combining High Specificity and Potent Cytotoxicity” Immunological Reviews 62:185-216 (February 1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimido-methyl)cy-clohexane-1-carboxylic acid. The carboxyl groups can be activated by combining them with succinimide or 1-hydroxyl-2-nitro-4-sulfonic acid, sodium salt. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLoS ONE 2(5):e415.) Alternatively, peptide immunogens can be linked to at least one artificial T-cell epitope capable of binding a large proportion of MHC Class II molecules, such as the pan DR epitope (“PADRE”). Pan DR-binding peptides (PADREs described in U.S. Pat. No. 5,736,142, WO 95/07707, and Alexander, et al, Immunity, 1:751-761 (1994).

Active immunogens can be presented in multimeric form in which multiple copies of an immunogen (peptide of polypeptide) are presented on a carrier as a single covalent molecule. In some embodiments, the carrier includes various forms of the multiple Aβ/tau/alpha-synuclein polypeptide. For instance, the multiple Aβ/tau/alpha-synuclein polypeptide of the immunogen can include polypeptides that have the Aβ antigen, the tau antigen and alpha-synuclein antigen in different orders, or may be present with or without an intrapeptide linker and/or a linker to a carrier.

In some compositions, the immunogenic peptides can also be expressed as fusion proteins with carriers. In certain compositions, the immunogenic peptides can be linked at the amino terminus, the carboxyl terminus, or internally to the carrier. In some compositions, the carrier is CRM197. In some compositions, the carrier is diphtheria toxoid.

### Nucleic Acids

The disclosure further provides nucleic acids encoding any of the amyloid-beta (Aβ) peptides, the tau peptides, and/or the alpha-synuclein peptides as disclosed herein. The nucleic acid immunotherapy compositions, as disclosed herein, comprise, consist of, or consist essentially of, a first nucleic acid sequence encoding an amyloid-beta (Aβ) peptide, a second nucleic acid sequence encoding a tau peptide, and/or a third nucleic acid sequence encoding an alpha-synuclein peptide as disclosed herein. For example, the Aβ peptide is a sequence that is 3-10 amino acid residues in length and from the first ten or 12 to 25 N-terminal residues of SEQ ID NO:01, and the tau peptide is a sequence that is 3-13 amino acids in length and from residues 244-400 of SEQ ID NO:02, and the alpha-synuclein peptide is a sequence that is 3-10 amino acids in length and from residues 81-140 of SEQ ID NO:58. Accordingly, a nucleic acid encoding any of SEQ ID NOS: 3-38 or 1002 to 1057 may be combined with a nucleic acid encoding any of SEQ ID NOS: 39-57, or 142-1000, and/or a nucleic acid encoding any of SEQ ID NOS: 59-129 to provide an immunogen and a component of pharmaceutical composition of the disclosure. Likewise, one or more nucleic acids encoding any of Aβ, tau and alpha-synuclein sequences may include the codons for an RR- N-terminal or -RR C-terminal dipeptide or polypeptide. In certain embodiments, the Aβ, tau and alpha-synuclein peptide sequences may be encoded by the same nucleic acid sequence or by separate nucleic acid sequences. In some embodiments, the nucleic acid sequences may also encode a linker to a carrier and/or a C-terminal cysteine as described herein. In addition, when a single nucleic acid sequence encodes both peptides, the sequence may also encode an intra-peptide linker as described herein. The nucleic acid compositions described herein (pharmaceutical compositions) can be used in methods for treating or effecting prophylaxis and/or prevention of Alzheimer's disease. In another embodiment, the nucleic acid immunotherapy compositions as disclosed herein provide compositions for reducing pathogenic forms of Aβ and/or tau and/or alpha-synuclein in the subject and/or in the tissue of the subject. In some embodiments, the Aβ and/or tau and/or alpha-synuclein reduced by the immunotherapy compositions is the pathological form(s) of the Aβ (e.g. extracellular plaque deposits of the β-amyloid peptide (Aβ); neuritic amyloid plaques), tau (e.g. flame-shaped neurofibrillary tangles of tau; neurofibrillary tangles of tau), and/or alpha-synuclein (e.g. oligomeric or fibrillar alpha-synuclein conglomerates and protofibrillar intermediates of alpha-synuclein oligomers). In yet other embodiment, pathological indicators of neurodegenerative disease and/or synucleinopathies are decreased by the nucleic acid immunotherapy compositions. In another embodiment, the nucleic acid immunotherapy compositions as disclosed herein provide compositions for reducing brain Aβ, brain tau and brain alpha-synuclein.

A nucleic acid such as DNA that encodes an immunogen and is used as a vaccine can be referred to as a “DNA immunogen” or “DNA vaccine” as the encoded polypeptides are expressed in vivo after administration of the DNA. DNA vaccines are intended to induce antibodies against the proteins of interest they encode in a subject by: integrating DNA encoding the proteins of interest into a vector (a plasmid or virus); administering the vector to the subject; and expressing the proteins of interest in the subject in which the vector has been administered to stimulate the immune system of the subject. A DNA vaccine remains in the body of the subject for a long time after the administration, and continues to slowly produce the encoded proteins. Thus, excessive immune responses can be avoided. DNA vaccines can also be modified using a genetic engineering techniques. Optionally, such nucleic acids further encode a signal peptide and can be expressed with the signal peptide linked to peptide. Coding sequences of nucleic acids can be operably linked with regulatory sequences to ensure expression of the coding sequences, such as a promoter, enhancer, ribosome binding site, transcription termination signal, and the like. The nucleic acids encoding Aβ, tau, and/or alpha-synuclein can occur in isolated form or can be cloned into one or more vectors. The nucleic acids can be synthesized by, for example, solid state synthesis or PCR of overlapping oligonucleotides. Nucleic acids encoding Aβ, tau, and/or alpha-synuclein peptides and polypeptides with and without linkers or cleavable linkers and with our without protein based carriers can be joined as one contiguous nucleic acid, e.g., within an expression vector.

DNA is more stable than RNA, but involves some potential safety risks such as induction of anti-DNA antibodies, thus in some embodiments, the nucleic acid can be RNA. RNA nucleic acid that encodes an immunogen and is used as a vaccine can be referred to as a “RNA immunogen” or “RNA vaccine” or “mRNA vaccine” as the encoded polypeptides are expressed in vivo after administration of the RNA. Ribonucleic acid (RNA) vaccines can safely direct a subject's cellular machinery to produce one or more polypeptide(s) of interest. In some embodiments, a RNA vaccine can be a non-replicating mRNA (messenger-RNA) or a virally derived, self-amplifying RNA. mRNA-based vaccines encode the antigens of interest and contain 5′ and 3′ untranslated regions (UTRs), whereas self-amplifying RNAs encode not only the antigens, but also the viral replication machinery that enables intracellular RNA amplification and abundant protein expression. In vitro transcribed mRNA can be produced from a linear DNA template using a T7, a T3 or an Sp6 phage RNA polymerase. The resulting product can contain an open reading frame that encodes the peptides of interest as disclosed herein, flanking 5′- and 3′-UTR sequences, a 5′ cap and a poly(A) tail. In some embodiments, a RNA vaccine can comprise trans-amplifying RNA (for example, see Beissert et al., Molecular Therapy January 2020 28(1):119-128). In certain embodiments, RNA vaccines encode an Aβ peptide and a tau peptide as disclosed herein, and are capable of expressing the Aβ and a tau peptides, in particular if transferred into a cell such as an immature antigen presenting cell. RNA may also contain sequences which encode other polypeptide sequences such as immune stimulating elements. In some embodiments, the RNA of a RNA vaccine can be modified RNA. The term “modified” in the context of the RNA can include any modification of RNA which is not naturally present in RNA. For example, modified RNA can refer to RNA with a 5′-cap; however, RNA may comprise further modifications. A 5′-cap can be modified to possess the ability to stabilize RNA when attached thereto. In certain embodiments, a further modification may be an extension or truncation of the naturally occurring poly(A) tail or an alteration of the 5′- or 3′-untranslated regions (UTR). In some embodiments, the RNA e.g. or mRNA vaccine is formulated in an effective amount to produce an antigen specific immune response in a subject. For example, the RNA vaccine formulation is administered to a subject in order to stimulate the humoral and/or cellular immune system of the subject against the Aβ, tau and alpha synuclein antigens, and thus may further comprise one or more adjuvant(s), diluents, carriers, and/or excipients, and is applied to the subject in any suitable route in order to elicit a protective and/or therapeutic immune reaction against the Aβ, tau and alpha synuclein antigens.

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

Techniques for the manipulation of nucleic acids, such as, e.g., generating mutations in sequences, sub-cloning, labeling probes, sequencing, hybridization and the like are well described in the scientific and patent literature. See, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

### Pharmaceutical Compositions

Each of the peptides and immunogens described herein can be presented in a pharmaceutical composition that is administered with pharmaceutically acceptable adjuvants and pharmaceutically acceptable excipients. The adjuvant increases the titer of induced antibodies and/or the binding affinity of induced antibodies relative to the situation if the peptide were used alone. A variety of adjuvants can be used in combination with an immunogen of the disclosure to elicit an immune response. Some adjuvants augment the intrinsic response to an immunogen without causing conformational changes in the immunogen that affect the qualitative form of the response. An adjuvant may be a natural compound, a modified version of or derivative of a natural compound, or a synthetic compound.

Some adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). As used herein, MPL refers to natural and synthetic versions of MPL. Examples of synthetic versions include PHAD®, 3D-PHAD® and 3D(6A)-PHAD® (Avanti Polar Lipids, Alabaster, Alabama).

QS-21 is a triterpene glycoside or saponin isolated from the bark of the Quillaja saponaria Molina tree found in South America (see Kensil et al., in Vaccine Design: The Subunit and Adjuvant Approach (eds. Powell & Newman, Plenum Press, NY, 1995)) QS-21 products include Stimulon® (Antigenics, Inc., New York, NY; now Agenus, Inc. Lexington, MA) and QS-21 Vaccine Adjuvant (Desert King, San Diego, CA). QS-21 has been disclosed, characterized, and evaluated in U.S. Pat. Nos. 5,057,540, and 8,034,348, the disclosures of which are herein incorporated by reference. Additionally, QS-21 has been evaluated in numerous clinical trials in various dosages. See, NCT00960531 (clinicaltrials.gov/ct2/show/study/NCT00960531), Hüll et al., Curr Alzheimer Res. 2017 July; 14(7): 696-708 (evaluated 50 mcg of QS-21 in with various doses of vaccine ACC-001); Gilman et al., “Clinical effects of Abeta immunization (AN1792) in patients with AD in an interrupted trial” Neurology. 2005 May 10; 64(9):1553-62; Wald et al., “Safety and immunogenicity of long HSV-2 peptides complexed with rhHsc70 in HSV-2 seropositive persons” Vaccine 2011; 29(47):8520-8529; and Cunningham et al., “Efficacy of the Herpes Zoster Subunit Vaccine in Adults 70 Years of Age or Older.” NEJM 2016 Sep. 15; 375(11):1019-32. QS-21 is used in FDA approved vaccines including SHINGRIX. SHINGRIX contains 50 mcg of QS-21. In certain embodiments, the amount of QS-21 is from about 10 μg to about 500 μg.

TQL1055 is an analogue of QS-21 (Adjuvance Technologies, Lincoln, NE). The semi-synthetic TQL1055 has been characterized in comparison to QS-21 as having high purity, increased stability, decreased local tolerability, decreased systemic tolerability. TQL1055 has been disclosed, characterized, and evaluated in US20180327436 A1, WO2018191598 A1, WO2018200656 A1, and WO2019079160 A1, the disclosures of which are herein incorporated by reference. US20180327436 A1 teaches that 2.5 fold more TQ1055 was superior to 20 μg QS-21 but there was not an improvement over 50 μg TQ1055. However, unlike QS-21 there was no increase in either weight loss or hemolysis of RBC as the TQL1055 dose increased. WO2018200656 A1 teaches that with an optimal amount of TQ1055, one can lower the amount of antigen and achieve superior titers. In certain embodiments, the amount of TQL1055 is from about 10 μg to about 500 μg.

Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Other adjuvants can be CpG oligonucleotides (see WO 98/40100), cytokines (e.g., IL-1, IL-1 alpha and β peptides, IL-2, γ-INF, IL-10, GM-CSF), chemokines (e.g., MIP1-α and β, and RANTES), saponins, RNA, and/or TLR agonists (for example, TLR4 agonists such as MPL and synthetic MPL molecules), aminoalkyl glucosaminide phosphate and other TLR agonists. Adjuvants can be administered as a component of a therapeutic composition with an active agent or can be administered separately, before, concurrently with, or after administration of the therapeutic agent.

In various embodiments of the disclosure, the adjuvant is QS-21 (Stimulon™). In some compositions, the adjuvant is MPL. In certain embodiments, the amount of MPL is from about 10 μg to about 500 μg. In some compositions, the adjuvant is TQL1055. In certain embodiments, the amount of TQL1055 is from about 10 μg to about 500 μg. In some compositions, the adjuvant is QS21. In certain embodiments, the amount of QS21 is from about 10 μg to about 500 μg. In some compositions, the adjuvant is a combination of MPL and QS-21. In some compositions, the adjuvant is a combination of MPL and TQL1055. In some compositions, the adjuvant can be in a liposomal formulation.

In addition, some embodiments of the disclosure can comprise a multiple antigen presenting system (MAP). Multiple antigen-presenting peptide vaccine systems have been developed to avoid the adverse effects associated with conventional vaccines (i.e., live-attenuated, killed or inactivated pathogens), carrier proteins and cytotoxic adjuvants. Two main approaches have been used to develop multiple antigen presenting peptide vaccine systems: (1) the addition of functional components, e.g., T-cell epitopes, cell-penetrating peptides, and lipophilic moieties; and (2) synthetic approaches using size-defined nanomaterials, e.g., self-assembling peptides, non-peptidic dendrimers, and gold nanoparticles, as antigen-displaying platforms. Use of a multiple antigenic peptide (MAP) system can improve the sometimes poor immunogenicity of subunit peptide vaccines. In a MAP system, multiple copies of antigenic peptides are simultaneously bound to the a- and ε-amino groups of a non-immunogenic Lys-based dendritic scaffold, helping to confer stability from degradation, thus enhancing molecular recognition by immune cells, and induction of stronger immune responses compared with small antigenic peptides alone. In some compositions, the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the peptides of the disclosure can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, peptide compositions can be in lyophilized form for constitution with a suitable vehicle, e.g., sterile pyrogen-free water, before use.

Peptides (and optionally a carrier fused to the peptide(s)) can also be administered in the form of a nucleic acid encoding the peptide(s) and expressed in situ in a subject. A nucleic acid segment encoding an immunogen is typically linked to regulatory elements, such as a promoter and enhancer that allow expression of the DNA segment in the intended target cells of a subject. For expression in blood cells, as is desirable for induction of an immune response, promoter and enhancer elements from, for example, light or heavy chain immunoglobulin genes or the CMV major intermediate early promoter and enhancer are suitable to direct expression. The linked regulatory elements and coding sequences are often cloned into a vector.

DNA and RNA can be delivered in naked form (i.e., without colloidal or encapsulating materials). Alternatively a number of viral vector systems can be used including retroviral systems (see, e.g., Boris-Lawrie and Teumin, Cur. Opin. Genet. Develop. 3(1):102-109 (1993)); adenoviral vectors (see, e.g., Bett et al, J. Virol. 67(10); 5911-21 (1993)); adeno-associated virus vectors (see, e.g., Zhou et al., J. Exp. Med. 179(6):1867-75 (1994)), viral vectors from the pox family including vaccinia virus and the avian pox viruses, viral vectors from the alpha virus genus such as those derived from Sindbis and Semliki Forest Viruses (see, e.g., Dubensky et al., J Virol. 70(1):508-519 (1996)), Venezuelan equine encephalitis virus (see U.S. Pat. No. 5,643,576) and rhabdoviruses, such as vesicular stomatitis virus (see WO 96/34625) and papillomaviruses (WO 94/12629; Ohe et al., Human Gene Therapy 6(3):325-333 (1995); and Xiao & Brandsma, Nucleic Acids. Res. 24(13):2620-2622 (1996)).

DNA and RNA encoding an immunogen, or a vector containing the same, can be packaged into liposomes, nanoparticles or lipoproteins complexes. Suitable other polymers, include, for example, protamine liposomes, polysaccharide particles, cationic nanoemulsion, cationic polymer, cationic polymer liposome, cationic lipid nanoparticles, cationic lipid, cholesterol nanoparticles, cationic lipid-cholesterol, PEG nanoparticle, or dendrimer nanoparticles. Additional suitable lipids and related analogs are described by U.S. Pat. Nos. 5,208,036, 5,264,618, 5,279,833, and 5,283,185, each of which are herein incorporated by reference in their entirety. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap. March-April 1997; 14(2):197-210).

Pharmaceutically acceptable carrier compositions can also include additives, including, but not limited to, water, pharmaceutically acceptable organic solvents, collagen, polyvinyl alcohol, polyvinylpyrrolidone, carboxyvinyl polymers, carboxymethylcellulose sodium, sodium polyacrylate, sodium alginate, water-soluble dextran, carboxymethyl starch sodium, pectin, methylcellulose, ethylcellulose, xanthan gum, gum arabic, casein, agar, polyethylene glycol, diglycerine, glycerine, propylene glycol, petrolatum, paraffin, stearyl alcohol, stearic acid, human serum albumin, mannitol, sorbitol, lactose, and surfactants acceptable as pharmaceutical additives.

### Subjects Amenable to Treatment

The presence of Aβ plaques and/or neurofibrillary tangles has been found in several diseases including Alzheimer's disease, Down's syndrome, mild cognitive impairment, cerebral amyloid angiopathy, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer's disease (LBVAD), chronic traumatic encephalopathy (CTE), globular glial tauopathy (GGT), Parkinson's disease, progressive supranuclear palsy (PSP), dry age-related macular degeneration (AMD), and inclusion-body myositis.

The compositions and methods of the disclosure can be used in treatment or prophylaxis of any of these diseases. Because of the widespread association between neurological diseases and Aβ and/or tau and/or alpha-synuclein, the compositions and methods of the disclosure can be used in treatment or prophylaxis of any subject showing elevated levels of Aβ and/or tau and/or alpha-synuclein (e.g., in the CSF) compared with a mean value in individuals without neurological disease. The compositions and methods of the disclosure can also be used in treatment or prophylaxis of neurological disease in individuals having a mutation in Aβ and/or tau and/or alpha-synuclein associated with neurological disease. The methods are particularly suitable for treatment or prophylaxis of Alzheimer's disease.

Subjects amenable to treatment include individuals at risk of disease but not showing symptoms, as well as patients presently showing symptoms, including treatment naïve subjects that have not been previous treated for disease. Subjects at risk of disease include those in an aging population, asymptomatic subjects with Aβ and/or tau and/or alpha-synuclein pathologies and having a known genetic risk of disease. Such individuals include those having relatives who have experienced this disease, and those whose risk is determined by analysis of genetic or biochemical markers. Genetic markers of risk include mutations in Aβ and/or tau and/or alpha-synuclein, as well as mutations in other genes associated with neurological disease. For example, the ApoE4 allele in heterozygous and even more so in homozygous form is associated with risk of Alzheimer's disease (AD). Other markers of risk of Alzheimer's disease include mutations in the APP gene, particularly mutations at position 717 and positions 670 and 671 referred to as the Hardy and Swedish mutations respectively, mutations in the presenilin genes, PS1 and PS2, a family history of Aβ, hypercholesterolemia or atherosclerosis. Individuals presently suffering from Alzheimer's disease can be recognized by PET imaging, from characteristic dementia, as well as the presence of risk factors described above. In addition, a number of diagnostic tests are available for identifying individuals who have AD. These include measurement of CSF or blood tau or phospho-tau and Aβ42 levels. Elevated tau or phospho-tau and decreased Aβ42 levels signify the presence of AD. Some mutations associated with Parkinson's disease, for example, Ala30Pro or Ala53Thr, or mutations in other genes associated with Parkinson's disease such as leucine-rich repeat kinase (LRRK2 or PARK8). Subjects can also be diagnosed with any of the neurological diseases mentioned above by the criteria of the DSM IV TR.

In asymptomatic subjects, treatment can begin at any age (e.g., 10, 20, 30, or more). Usually, however, it is not necessary to begin treatment until a subject reaches 20, 30, 40, 50, 60, 70, 80, or 90 years of age. Treatment typically entails multiple dosages over a period of time. Treatment can be monitored by assaying antibody levels over time. If the response falls, a booster dosage is indicated. In the case of potential Down's syndrome patients, treatment can begin antenatally by administering therapeutic agent to the mother or shortly after birth.

### Methods of Treatments and Uses

The disclosure provides methods of inhibiting or reducing aggregation of Abeta and/or tau and/or alpha-synuclein in a subject having or at risk of developing a neurodegenerative disease (e.g., Alzheimer's disease). The methods include administering to the subject the compositions as disclosed herein. A therapeutically effective amount is a dosage that, when given for an effective period of time, achieves the desired immunological or clinical effect. Dosage regimens may be adjusted to provide the optimum therapeutic response. For example, several divided doses may be administered at set intervals (e.g., weekly, monthly) or the dose may be proportionally reduced as indicated by the exigencies of the therapeutic situation.

In prophylactic applications, the compositions described herein can be administered to a subject susceptible to, or otherwise at risk of a disease (e.g., Alzheimer's disease) in a regimen (dose, frequency and route of administration) effective to reduce the risk, lessen the severity, or delay the onset of at least one sign or symptom of the disease. In particular, the regimen is effective to inhibit or delay Aβ plaque formation and/or inhibit or delay tau or phospho-tau and paired filaments and/or alpha-synuclein synucleinopathies formed from it in the brain, and/or inhibit or delay its toxic effects and/or inhibit/or delay development of behavioral deficits. In therapeutic applications, the compositions described herein are administered to a subject suspected of, or a patient already suffering from a disease (e.g., Alzheimer's disease) in a regimen (dose, frequency and route of administration) effective to ameliorate or at least inhibit further deterioration of at least one sign or symptom of the disease. In particular, the regimen is preferably effective to reduce or at least inhibit further increase of levels of Aβ plaques and/or tau, phosphor-tau, or paired filaments formed from it, and/or alpha-synuclein synucleinopathies, and associated toxicities and/or behavioral deficits.

A regimen is considered therapeutically or prophylactically effective if an individual treated achieves an outcome more favorable than the mean outcome in a control population of comparable subjects not treated by methods of the invention, or if a more favorable outcome is demonstrated in treated subjects versus control subjects in a controlled clinical trial (e.g., a phase II, phase II/III or phase III trial) at the p<0.05 or 0.01 or even 0.001 level.

Effective doses of vary depending on many different factors, such as means of administration, target site, physiological state of the patient, whether the patient is an ApoE carrier, whether the patient is human or an animal, other medications administered, and whether treatment is prophylactic or therapeutic.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times. In some embodiments, a RNA (e.g., mRNA) vaccine is administered to a subject by intradermal, intramuscular injection, or by intranasal administration.

In some embodiments, the amount of an agent for active immunotherapy varies from 1 to 1,000 micrograms (μg), or from 0.1-500 μg, or from 10 to 500 μg, or from 50 to 250 μg per patient and can be from 1-100 or 1-10 μg per injection for human administration. The timing of injections can vary significantly from once a day, to once a week, to once a month, to once a year, to once a decade. A typical regimen consists of an immunization followed by booster injections at time intervals, such as 6 week intervals or two months. Another regimen consists of an immunization followed by one or more booster injections 1, 2, 3, 4, 5, 6, 7, 8, 9, 10, 11, or 12 months later. Another regimen entails an injection every two months for life. Alternatively, booster injections can be on an irregular basis as indicated by monitoring of immune response. The frequency of administration may be once or more as long as the side effects are within a clinically acceptable range.

In some embodiments, the compositions or methods as disclosed herein comprise administering to a subject a nucleic acid vaccine comprising one or more DNA or RNA polynucleotides having an open reading frame encoding a first peptide and a second peptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid is administered to the subject by intradermal or intramuscular injection. In some embodiments, the nucleic acid is administered to the subject on day zero. In some embodiments, a second dose of the nucleic acid is administered to the subject on day seven, or fourteen, or twenty one.

The compositions described herein are preferably administered via a peripheral route (i.e., one in which the administered composition results in a robust immune response and/or the induced antibody population crosses the blood brain barrier to reach an intended site in the brain, spinal cord, or eye). For peripheral diseases, the induced antibodies leave the vasculature to reach the intended peripheral organs. Routes of administration include oral, subcutaneous, intranasal, intradermal, or intramuscular. Some routes for active immunization are subcutaneous and intramuscular. Intramuscular administration and subcutaneous administration can be made at a single site or multiple sites. Intramuscular injection is most typically performed in the arm or leg muscles. In some methods, agents are injected directly into a particular tissue where deposits have accumulated.

The number of dosages administered can be adjusted to result in a more robust immune response (for example, higher titers). For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. For chronic disorders, a vaccine/immunotherapy as disclosed herein can be administered at regular intervals, e.g., weekly, fortnightly, monthly, quarterly, every six months for at least 1, 5 or 10 years, or the life of the patient.

An effective amount of a DNA or RNA encoded immunogen can be between about 1 nanogram and about 1 gram per kilogram of body weight of the recipient, or about between about 0.1 μg/kg and about 10 mg/kg, or about between about 1 μg/kg and about 1 mg/kg. Dosage forms suitable for internal administration preferably contain (for the latter dose range) from about 0.1 μg to 100 μg of active ingredient per unit. The active ingredient may vary from 0.5 to 95% by weight based on the total weight of the composition. Alternatively, an effective dose of dendritic cells loaded with the antigen is between about 10⁴ and 10⁸ cells. Those skilled in the art of immunotherapy will be able to adjust these doses without undue experimentation.

The nucleic acid compositions may be administered in a convenient manner, e.g., injection by a convenient and effective route. Routes can include, but are not limited to, intradermal “gene gun” delivery or intramuscular injection. The modified dendritic cells are administered by subcutaneous, intravenous or intramuscular routes. Other possible routes include oral administration, intrathecal, inhalation, transdermal application, or rectal administration.

Depending on the route of administration, the composition may be coated in a material to protect the compound from the action of enzymes, acids and other natural conditions which may inactivate the compound. Thus, it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol) or in an appropriate carrier such as liposomes (including water-in-oil-in-water emulsions) as well as conventional liposomes (Strejan et al., J. Neuroimmunol 7(1):27-41, 1984).

The immunotherapeutic compositions disclosed herein may also be used in combination with other treatments for diseases associated with the accumulation of Aβ and/or tau and/or alpha-synuclein, for example, anti-Aβ antibodies such as antibodies that specifically bind to any of the Aβ epitopes disclosed herein. For example, aducanumab or any of the antibodies disclosed in, for example, U.S. Patent Publication No. 20100202968 and U.S. Pat. No. 8,906,367, and/or anti-tau antibodies such as antibodies that specifically bind to any of the tau epitopes disclosed herein, ABBV-8E12, gosuranemab, zagotenemab, RG-6100, BIIB076 or any of the antibodies disclosed in WO2014/165271, U.S. Pat. No. 10,501,531, WO2017/191560, US2019/0330314, WO2017/191561, US2019/0330316, WO2017/191559, and WO2018/204546; and/or anti-alpha-synuclein antibodies such as antibodies that specifically bind to any of the alpha-synuclein epitopes disclosed herein, or antibodies and/or other alpha-synuclein binding compounds, such as, PRX002/RO7046015, PRX002/RG7935 (Prasinezumab), NPT200-11/UCB0599, NPT088, BIIB054 (Cinpanemab), ABBV-0805, MEDI-1341, NPT088, Lu AF82422. In some combination therapy methods, the patient receives passive immunotherapy prior to the active immunotherapy methods disclosed herein. In other methods, the patient receives passive and active immunotherapy during the same period of treatment. Alternatively, patients may receive active immunotherapy prior to passive immunotherapy. Combinations may also include small molecule therapies and non-immunogenic therapies such as RAZADYNE® (galantamine), EXELON® (rivastigmine), and ARICEPT® (donepezil) and other compositions that improve the function of nerve cells in the brain.

The compositions of the disclosure may be used in the manufacture of medicaments for the treatment regimens described herein.

### Treatment Regimens

Desired outcomes of the methods of treatment as disclosed herein vary according to the disease and patient profile and are determinable to those skilled in the art. Desired outcomes include an improvement in the patient's health status. Generally, desired outcomes include measurable indices such as reduction or clearance of pathologic amyloid fibrils, decreased or inhibited amyloid aggregation and/or deposition of amyloid fibrils, reduction or clearance of Aβ plaque formation and/or inhibit or delay tau or phospho-tau and paired filaments and/or decreased alpha-synuclein synucleinopathies, and increased immune response to pathologic and/or aggregated amyloid fibrils. Desired outcomes also include amelioration of amyloid disease-specific symptoms. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same amyloid disease as the individual being treated, who is about the same age as the individual being treated (to ensure that the stages of the disease in the treated individual and the control individual are comparable), but who has not received treatment using the disclosed immunotherapy/vaccine formulations. Alternatively, a control individual is a healthy individual, who is about the same age as the individual being treated. Changes or improvements in response to therapy are generally statistically significant and described by a p-value less than or equal to 0.1, less than 0.05, less than 0.01, less than 0.005, or less than 0.001 may be regarded as significant.

Effective doses of the compositions as disclosed herein, for the treatment of a subject vary depending upon many different factors, including means of administration, target site, physiological state of the patient, whether the patient is human or an animal, other medications administered, if any, and whether treatment is prophylactic or therapeutic. Treatment dosages can be titrated to optimize safety and efficacy. The amount of immunogen can also depend on whether adjuvant is also administered, with higher dosages being required in the absence of adjuvant. The amount of an immunogen for administration sometimes varies from 1-500 μg per patient and more usually from 5-500 μg per injection for human administration. Occasionally, a higher dose of 1-2 mg per dosage is used. Typically, about 10, 20, 50 or 100 μg is used for each human dosage. The timing of dosages can vary significantly from once a day, to once a year, to once a decade. On any given day that a dosage of immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster dosage(s) at 6-week intervals. Another regimen consists of an immunization followed by booster dosage(s) 1, 2, 3, 4, 5, 6, 7, 8, 9, 10, 11, or 12 months later. Another regimen entails dosage(s) every two months for life. Alternatively, booster dosage(s) can be on an irregular basis as indicated by monitoring of immune response.

When administered in combination with a second treatment for Alzheimer's disease, such as, Razadyne® (galantamine), Exelon® (rivastigmine), and Aricept® (donepezil), the second treatment can be administered according the product label or as necessary in view of the treatment with the compositions of the disclosure.

### Kits

The disclosure further provides kits (e.g., containers) comprising the compositions disclosed herein and related materials, such as instructions for use (e.g., package insert). The instructions for use may contain, for example, instructions for administration of the compositions and optionally one or more additional agents. The containers of peptide and/or nucleic acid compositions may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products. Kits can also include a second container comprising a pharmaceutically acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

### Uses

Each of the peptides, polypeptides, immunogens, and pharmaceutical compositions described herein may be for use in treating one or more of the diseases as described herein. In addition, each of the peptides, polypeptides, immunogens, and pharmaceutical compositions described herein may be for use in methods for treating one or more of the diseases as described herein. Each of the peptides, polypeptides, immunogens, and pharmaceutical compositions described herein may be used in a method for manufacturing a medicament for treating or use in treating one or more of the diseases as described herein.

The following are provided for exemplification purposes only and are not intended to limit the scope of the invention described in broad terms above.

All U.S. and international patent applications identified herein are incorporated by reference in their entirety.

### EXAMPLES

### Example 1: Animal Immunizations

Female Swiss Webster mice are injected subcutaneously at two sites with 100 μl (200 μl in total) of immunogen on day 0, 14, and 28. Immunogens are prepared by combining 25 μg of test immunogen and 25 μg of QS21 adjuvant in 200 μl phosphate buffered saline (PBS). Mice are bled at least on day 21 and 35 by nicking tails and collecting 50 μl of blood, followed by processing to serum. The immunogens tested include:

TABLE 3
Immunogens comprising
Aβ/tau/alpha-synuclein peptides

| Immunogen | Immunogen sequence (SEQ ID NO.) |
|---|---|
| 24 | DAEFRHDRRPDNEAYERRQIVYKPVKKC |
| | (SEQ ID NO: 130) |
| 25 | DAEFRHDRRQIVYKPVRRPDNEAYEKKC |
| | (SEQ ID NO: 131) |
| 26 | DAEFRHDRRPDNEAYERRNIKHVPGKKC |
| | (SEQ ID NO: 132) |
| 27 | DAEFRHDRRNIKHVPGRRPDNEAYEKKC |
| | (SEQ ID NO: 133) |
| 14 | DAEFRHDRRQIVYKPVRRPDNEAYERRN |
| | IKHVPGGC (SEQ ID NO: 134) |
| 15 | DAEFRHDRRDPDNEAYRRNIKHVPGRRQ |
| | IVYKPVGGC (SEQ ID NO: 135) |
| 16 | blocked amine-EFRHDSGRRQIVYK |
| | PVRRPDNEAYERRNIKHVPGGC (SEQ |
| | ID NO: 136) |
| 17 | blocked amine-EFRHDSGRRDPDNE |
| | AYRRNIKHVPGRRQIVYKPVGGC (SEQ |
| | ID NO: 137) |
| Tri 2 | DAEFRHDRRDPDNEAYERRENLKHQPGG |
| | GC (SEQ ID NO: 1058) |
| Tri 1 | DAEFRHDRRENLKHQPGRRDPDNEAYEG |
| | GC (SEQ ID NO: 1059) |
| Tri 4 | DAEFRHDRRPDNEAYERRENLKHQPGGG |
| | C (SEQ ID NO: 1060) |
| Tri 3 | DAEFRHDRRENLKHQPGRRPDNEAYEGG |
| | C (SEQ ID NO: 1061) |
| Tri 5 | DAEFRHDRRSKIGSKDNIKHRRDPDNEA |
| | YEGGC (SEQ ID NO: 1062) |
| Tri 6 | DAEFRHDRRDPDNEAYERRSKIGSKDNI |
| | KHGGC (SEQ ID NO: 1063) |

Immunogens comprise Aβ/tau/alpha-synuclein peptides, a C-terminal linker, and a C-terminal cysteine, and are coupled through the C-terminal cysteine to CRM-197 with a maleimide linkage.

Guinea pigs were injected intramuscularly with 50 μg of a test immunogen, 25 μg QS21 in 200 μl of Addavax on day 0, 21, 49 and 77. Bleeds were done starting 7 days post immunization. The immunogens tested are shown in Table 3. The immunogens peptides are coupled through the C-terminal cysteine to CRM-197 with a maleimide linkage.

Female Guinea Pigs were at least 5 weeks old at the start of the study having an approximate body weight of 350-500 g. Appropriate animal housing and research procedures for animal husbandry and care were conducted in an accredited facility in accordance with the guidelines of the U.S. Department of Agriculture's (USDA) and the Assessment and Accreditation of Laboratory Animal Care (AAALAC) International.

The immunogen concentration was 0.5 mg/ml. Prior to each administration of the test immunogen, approximately a 3 cm² area on each hind limb was shaved and wiped with ethanol for visualization of the injection site. Each animal received a test immunogen dose of 200 microliters (0.25 micrograms/microliter) divided into two separate sites each of 100 microliter per injection (i.e., animals received 50 μg of immunogen in 100 μl PBS+25 μg of QS21 in 100 μl MF59). A 25G-27G needle was inserted intramuscularly into the hind limb, approximately 0.25-0.5 cm deep, and injected at 100 microliters per site. Injection sites were rotated each administration between four separate sites per hind limb and separated by at least 2 cm.

### Example 2: Measurement of Antibody Titers

Whole blood samples were collected into clot activator tubes via jugular vein at 250-350 microliters per collection at weeks 1, 4, 8 and 12 for Guinea pigs and 50 microliters per collection at weeks 1, 3, 7 and 11 by nicking tails for mice. The maximum volume of whole blood was collected into clot activator tubes via cardiac puncture at termination on the final collection week. All blood samples were allowed to clot at room temperature for greater than 30 minutes, centrifuged at ambient temperature (approximately 20-25° C.) at 3,000 RPM for 10-15 minutes, and serum supernatant was transferred individually into clean cryovials. Serum supernatant was stored frozen at −80° C. (±12° C.).

### Aβ Titers (Mice)

2 μg/ml Aβ 1-28 monomers are coated at coated on to the plate 100 μl per well in PBS and incubated overnight at room temperature. Plates were blocked for 1 hour with 1% BSA in PBS. Plates were aspirated, and to row A 200 μl of 0.1% BSA in PBS Tween was added. In column 1, negative mouse serum was added at 1/100 while the rest of the row contained 1/100 test serums. Rows were serially diluted ½ down the plate giving dilution of 1/100 to 1/12800. Wells were incubated 2 hours at room temperature then were washed. A 1/5000 dilution of anti-mouse IgG HRP in 0.1% BSA in PBS Tween was prepared and then 100 μl added to the washed well. This incubated for 1 hour and was washed. OPD substrate was prepared using Thermo-Fisher OPD tablets at 1 tablet per 10 mL. Thermo-Fisher substrate buffer was added at 1/10, and each well had 100 μl added and was incubated for 15 minutes. 50 μl of 2N H₂SO₄ was added to stop the reaction and plates were read on a Molecular Devices Spectromax at 490 nM. Titer was defined as the dilution giving 50% maximum OD and were extrapolated if it fell between dilutions.

### Tau Titers (Mice)

2 μg/ml recombinant WT Tau 4R2N was coated on to the plate using 100 μl per well in PBS and incubated overnight at room temperature. Plates were blocked for 1 hour with 1% BSA in PBS. Plates were aspirated and to row A 200 μl of 0.1% BSA in PBS Tween was added. In column 1, negative mouse serum was added at 1/100 while the rest of the row contained 1/100 test sera. Rows were serially diluted ½ down the plate giving dilution of 1/100 to 1/12800. Wells were incubated 2 hours at room temperature then were washed. A 1/5000 dilution of anti-mouse IgG HRP in 0.1% BSA in PBS Tween was prepared and then 100 μl added to the washed well. This reaction mixture was incubated for 1 hour and was washed. OPD substrate was prepared using Thermo-Fisher OPD tablets at 1 tablet per 10 mL. Thermo-Fisher substrate buffer was added at 1/10 and each well had 100 μl added and was incubated for 15 minutes. 50 μl of 2N H₂SO₄ was added to stop the reaction and plates were read at 490 nm on a Molecular Devices Spectromax. Titer was defined as the dilution giving 50% maximum OD measurement and was extrapolated if it fell between dilutions.

### Alpha-Synuclein Titers (Mice)

2 μg/ml recombinant human alpha-synuclein was coated on to the plate 100 μl per well in PBS and incubated overnight at room temperature. Plates were blocked for 1 hour with 1% BSA in PBS. Plates were aspirated and to row A 200 μl of 0.1% BSA in PBS Tween was added. In column 1, negative mouse serum was added at 1/100 while the rest of the row contained 1/100 test serums. Rows were serially diluted ½ down the plate giving dilution of 1/100 to 1/12800. Wells incubated 2 hours at room temperature then were washed and a 1/5000 dilution of anti-mouse IgG HRP in 0.1% BSA in PBS Tween was prepared and then 100 μl added to the washed well. This incubated for 1 hour and was washed. OPD substrate was prepared using Thermo-Fisher OPD tablets at 1 tablet per 10 mL. Thermo-Fisher substrate buffer was added at 1/10 and each well had 100 μl added and was incubated for 15 minutes. 50 μl of 2N H₂SO₄ was added to stop the reaction and plates were read on a Molecular Devices Spectromax at 490 nm. Titer was defined as the dilution giving 50% maximum OD and was extrapolated if it fell between dilutions.

### Aβ Titers (Guinea Pig)

2 μg/ml Aβ 1-28 monomers were coated onto the plate at 100 μl per well in PBS and incubated overnight at room temperature. Plates were blocked for 1 hour with 1% BSA in PBS. Plates were re-aspirated, and to row A 200 μL of 0.1% BSA in PBS Tween was added. In column 1, negative Guinea Pig serum was added at 1/100 while the rest of the rows contained 1/100 test serums. Rows were serially diluted ½ down the plate giving dilution of 1/100 to 1/12800. Wells incubated 2 hours at room temperature then were washed and a 1/5000 dilution of anti-Guinea Pig IgG HRP in 0.1% BSA in PBS Tween was prepared and then 100 μl added to the washed well. This incubated for 1 hour and was washed. OPD substrate was prepared using Thermo-Fisher OPD tablets at 1 tablet per 10 mL. Thermo-Fisher substrate buffer was added at 1/10 and each well had 100 μl added and was incubated for 15 minutes. 50 μl of 2N H₂SO₄ was added to stop the reaction and plates were read on a Molecular Devices Spectromax at 490 nm. Titer was defined as the dilution giving 50% maximum OD and was extrapolated if it fell between dilutions.

### Tau Titers (Guinea Pig)

2 μg/ml recombinant wild-type Tau 4R2N was coated on to the plate at 100 μl per well in PBS and incubated overnight at room temperature. Plates were blocked for 1 hour with 1% BSA in PBS. Plates were aspirated, and to row A 200 μl of 0.1% BSA in PBS Tween was added. In column 1, negative Rabbit serum was added at 1/100 while the rest of the row contained 1/100 test serums. Rows were serially diluted ½ down the plate giving dilution of 1/100 to 1/12800. Wells were incubated 2 hours at room temperature then washed. A 1/5000 dilution of anti-rabbit IgG HRP in 0.1% BSA in PBS Tween was prepared and then 100 μl added to the washed well. This mixture was incubated for 1 hour and then washed. OPD substrate was prepared using Thermo-Fisher OPD tablets at 1 tablet per 10 mL. Thermo-Fisher substrate buffer was added at 1/10 (each well having 100 μl added) and was incubated for 15 minutes. 50 μl of 2N H₂SO₄ was added to stop the reaction and plates were read on a Molecular Devices Spectromax at 490 nm. Titer was defined as the dilution giving 50% maximum OD and was extrapolated if it fell between dilutions.

TABLE 4
Titers of mice vaccinated with one of 4 tri-peptide immunogens.

| | | |
|---|---|---|
| Aβ titer | Tau titer | AS titer |

Tri 1 immunogen:
DAEFRHDRRENLKHQPGRRDPDNEAYEGGC (SEQ ID NO: 1059)

| | | | |
|---|---|---|---|
| Mouse 1 | 20000 | 100 | 1000 |
| Mouse 2 | 12000 | 25 | 13000 |
| Mouse 3 | 5000 | 100 | 900 |
| Mouse 4 | 30000 | 12000 | 3300 |

Tri 2 immunogen:
DAEFRHDRRDPDNEAYERRENLKHQPGGGC (SEQ ID NO: 1058)

| | | | |
|---|---|---|---|
| Mouse 1 | 22000 | 400 | 1600 |
| Mouse 2 | 60000 | 6000 | 1000 |
| Mouse 3 | 25000 | 2200 | 1200 |
| Mouse 4 | 50000 | 6000 | 3300 |

Tri 3 immunogen:
DAEFRHDRRENLKHQPGRRPDNEAYEGGC (SEQ ID NO: 1061)

| | | | |
|---|---|---|---|
| Mouse 1 | 4000 | 25 | 13500 |
| Mouse 2 | 8000 | 600 | 2000 |
| Mouse 3 | 12000 | 25 | 3200 |
| Mouse 4 | 10000 | 25 | 8000 |

Tri 4 immunogen:
DAEFRHDRRPDNEAYERRENLKHQPGGGC (SEQ ID NO: 1060)

| | | | |
|---|---|---|---|
| Mouse 1 | 7000 | 25 | 15000 |
| Mouse 2 | 3200 | 25 | 7500 |
| Mouse 3 | 24000 | 1600 | 18000 |
| Mouse 4 | 400 | 25 | 18000 |

### Alpha-Synuclein Titers (Guinea Pig)

2 μg/ml recombinant human alpha synuclein was coated on to the plate at 100 μl per well in PBS and incubated overnight at room temperature. Plates were blocked for 1 hour with 1% BSA in PBS. Plates were re-aspirated, and to row A 200 μl of 0.1% BSA in PBS Tween was added. In column 1, negative Guinea pig serum was added at 1/100, while the rest of the row contained 1/100 test sera. Rows were serially diluted ½ down the plate giving dilution of 1/100 to 1/12800. Wells were incubated 2 hours at room temperature then washed. A 1/5000 dilution of anti-Guinea Pig IgG HRP in 0.1% BSA in PBS Tween was prepared and then 100 μl added to the washed well. This mixture was incubated for 1 hour and then washed. OPD substrate was prepared using Thermo-Fisher OPD tablets at 1 tablet per 10 mL. Thermo-Fisher substrate buffer was added at 1/10 (each well having 100 μl added) and the mixture was incubated for 15 minutes. 50 μl of 2N H₂SO₄ was added to stop the reaction and plates were read on a Molecular Devices Spectromax at 490 nm. Titer was defined as the dilution giving 50% maximum OD and was extrapolated if it fell between dilutions.

### Example 3: Staining of Alzheimer's Brain Tissue with Sera from Animals Immunized with Immunogens as Disclosed Herein

Autopsy blocks of fresh frozen human brain tissue (˜0.5 g) were embedded in optimal cutting temperature compound (OCT compound) and cut using a cryostat to generate 10 μm sections. The sections were placed into a solution of glucose oxidase and beta D-glucose, in the presence of sodium azide, to block endogenous peroxidase. Once tissue sections were prepared, the staining with the specified animal sera from animals immunized with a vaccine as disclosed herein was carried out at two dilutions (1:300 and 1:1500), using a species appropriate secondary antibody and a DAKO DAB Detection Kit as per the manufacturer's instructions. The staining was processed using an automated Leica Bond Stainer. The results indicate whether sera from animals immunized with a vaccine as disclosed herein comprise antibodies specific to Aβ, tau, and/or alpha-synuclein in human brain tissue of Alzheimer's patients.

### Example 4: Serum from Vaccinated Animals Blocks Soluble Aβ Aggregates from Binding to Neurons

E18 primary rat hippocampal neurons are cultured as described previously (Zago, et al. “Neutralization of Soluble, Synaptotoxic Amyloid β Species by Antibodies Is Epitope Specific,” J Neurosci. 2012 Feb. 22; 32(8): 2696-2702). Soluble Aβ aggregate was pre-incubated with or without animal vaccine serum on culture DIV14-21 to block soluble Aβ aggregate from neuritic binding. Animal serum was isolated from animals vaccinated with immunogen peptides as shown in Table 3. Fresh unlabeled, biotinylated or (9:1) soluble Aβ was prepared one day prior and incubated overnight at 4° C. Each diluted serum sample (1:1000, 1:300, and 1:100) and soluble Aβ solution was prepared at 2× the final concentration in one-half of final treatment volume using NeuroBasal-no phenol red (NB-NPR) medium. This was combined with one-half final volume of 2× soluble Aβ and with one-half final volume of 2× diluted animal vaccine serum to make up a 1× final concentration in total final treatment volume, which was mixed well and then pre-incubated for 30 minutes at 37° C. E18 neurons were rinsed with NB-NPR at 150 μL/well before adding binding treatment. Animal serum from vaccinated animals/AD treatment was added to E18 neurons at 60 μL/well, and then incubated for 30 minutes at 37° C. under normal incubator conditions (5% CO₂; 9% O₂). Cells were rinsed twice using 150 μL/well of NB-NPR, and then fixed in 4% paraformaldehyde in 1×DPBS for 20 minutes. Cells were permeabilized using 0.1 TX-100 for 5 minutes, and blocked using 10% normal goat serum (NGS) for 1 hour at room temperature (RT). Cells were incubated with MAP2 & NeuN primary antibodies in 100 μL/well, 1×DPBS containing 1% BSA+1% NGS overnight at 4° C. The next day, cells were rinsed twice in 150 μL/well 1×DPBS for 5 minutes each wash. Secondary antibodies were added for 1 hour at RT in 100 μL/well 1×DPBS+1% BSA+1% NGS. High-content imaging (HCl) analysis was performed to quantify soluble aggregate Aβ neuritic binding using Operetta HCl CLS instrument (Perkin Elmer; modified Neurite Outgrowth algorithm: 40× H₂O objective; 40 fields per well; (n=3) per condition; data shown as mean (+/−) SD); MAP2 & NeuN (Abcam) neuronal markers used to each trace neurite tree and count cell body number per optical field; Neuritic Aβ soluble aggregate spots detected using streptavidin-488 or polyclonal Aβ antibody (Thermo; Millipore); and data reported as Aβ soluble aggregate spots/neuron (or as Integrated Intensity)).

Approximately 80-150 neurons were observed per well for each condition tested. The results indicate whether animal serum from animals vaccinated with the immunogen peptides as disclosed herein comprise antibodies specific to Aβ that can block soluble Aβ aggregates from binding to neurons.

### Example 5: Serum from Vaccinated Animals Stains Aβ Plaques and/or Tau Pathologies and/or Synucleinopathies in Human Brain Tissue

Fresh frozen human brain tissues from autopsied Alzheimer's disease donors or non-diseased controls was embedded in OCT, and cut in a cryostat to generate 10 μm frozen sections. The tissue sections were incubated in a solution of glucose oxidase and beta D-glucose, in the presence of sodium azide, to block endogenous peroxidase. The staining with sera from vaccinated animals (animals were vaccinated with the immunogen peptides as disclosed in Table 3) or control animals were then carried out at 1:1000 dilution, in an automated Leica Bond Rx Stainer (Leica Biosystems). Antibody binding was detected using the Bond Polymer Refine Detection Kit (DS9800, Leica Biosystems), which is based on an anti-mouse polymer detection, DAB visualization and hematoxylin nuclear counter-staining. After cover-slipping, the stained tissue slides were digitally imaged with a Hamamatsu NanoZoomer 2.0HT slide scanner (Hamamatsu Corporation) with an NDP.scan, 2.5.85 software. The digitized images were viewed and analyzed using the NDP.view, 2.7.43.0 software.

Results demonstrate Aβ plaques and/or tau neurofibrillary tangles, and/or alpha-synuclein based synucleinopathies were identified based on their typical histopathological characteristics. Such pathologies were absent from tissues incubated with control animal serum. Non-diseased tissue had no such pathological staining after incubation with the sera from animals vaccinated with the immunogen peptides as disclosed herein. The immunogens disclosed herein produce Aβ antibody titers capable of causing phagocytosis of Aβ, and the synuclein titers are able to prevent the internalization of synuclein soluble aggregates into cells.

Although various specific embodiments of the present invention have been described herein, it is to be understood that the invention is not limited to those precise embodiments and that various changes or modifications can be affected therein by one skilled in the art without departing from the scope and spirit of the invention.

| SEQUENCES | |
|---|---|
| Abeta (Aβ) 1-42 | |
| SEQ ID NO: 01 | |
| DAEFRHDSGYEVHHQKLVFFAEDVGSNKGAIIGLMVGGVVIA | |
| TAU (UNIPROTKB-P10636 (Homo sapiens) | |
| >P10636-8 (Isoform Tau-F) | |
| SEQ ID NO: 02 | |
| 10 20 30 40 50 | |
| MAEPRQEFEV MEDHAGTYGL GDRKDQGGYT MHQDQEGDTD AGLKESPLQT | |
| 60 70 80 90 100 | |
| PTEDGSEEPG SETSDAKSTP TAEDVTAPLV DEGAPGKQAA AQPHTEIPEG | |
| 110 120 130 140 150 | |
| TTAEEAGIGD TPSLEDEAAG HVTQARMVSK SKDGTGSDDK KAKGADGKTK | |
| 160 170 180 190 200 | |
| IATPRGAAPP GQKGQANATR IPAKTPPAPK TPPSSGEPPK SGDRSGYSSP | |
| 210 220 230 240 250 | |
| GSPGTPGSRS RTPSLPTPPT REPKKVAVVR TPPKSPSSAK SRLQTAPVPM | |
| 260 270 280 290 300 | |
| PDLKNVKSKI GSTENLKHQP GGGKVQIINK KLDLSNVQSK CGSKDNIKHV | |
| 310 320 330 340 350 | |
| PGGGSVQIVY KPVDLSKVTS KCGSLGNIHH KPGGGQVEVK SEKLDFKDRV | |
| 360 370 380 390 400 | |
| QSKIGSLDNI THVPGGGNKK IETHKLTFRE NAKAKTDHGA EIVYKSPVVS | |
| 410 420 430 440 | |
| GDTSPRHLSN VSSTGSIDMV DSPQLATLAD EVSASLAKQG L | |
| A-beta immunogens: | |
| (SEQ ID NO: 03) | |
| DAEFRHDSGY | |
| (SEQ ID NO: 04) | |
| DAEFRHDSG | |
| (SEQ ID NO: 05) | |
| DAEFRHDS | |
| (SEQ ID NO: 06) | |
| DAEFRHD | |
| (SEQ ID NO: 07) | |
| DAEFRH | |
| (SEQ ID NO: 08) | |
| DAEFR | |
| (SEQ ID NO: 09) | |
| DAEF | |
| (SEQ ID NO: 10) | |
| DAE | |
| (SEQ ID NO: 11) | |
| AEFRHDSGY | |
| (SEQ ID NO: 12) | |
| AEFRHDSG | |
| (SEQ ID NO: 13) | |
| AEFRHDS | |
| (SEQ ID NO: 14) | |
| AEFRHD | |
| (SEQ ID NO: 15) | |
| AEFRH | |
| (SEQ ID NO: 16) | |
| AEFR | |
| (SEQ ID NO: 17) | |
| AEF | |
| (SEQ ID NO: 18) | |
| EFRHDSGY | |
| (SEQ ID NO: 19) | |
| EFRHDSG | |
| (SEQ ID NO: 20) | |
| EFRHDS | |
| (SEQ ID NO: 21) | |
| EFRHD | |
| (SEQ ID NO: 22) | |
| EFRH | |
| (SEQ ID NO: 23) | |
| EFR | |
| (SEQ ID NO: 24) | |
| FRHDSGY | |
| (SEQ ID NO: 25) | |
| FRHDSG | |
| (SEQ ID NO: 26) | |
| FRHDS | |
| (SEQ ID NO: 27) | |
| FRHD | |
| (SEQ ID NO: 28) | |
| FRH | |
| (SEQ ID NO: 29) | |
| RHDSGY | |
| (SEQ ID NO: 30) | |
| RHDSG | |
| (SEQ ID NO: 31) | |
| RHDS | |
| (SEQ ID NO: 32) | |
| RHD | |
| (SEQ ID NO: 33) | |
| HDSGY | |
| (SEQ ID NO: 34) | |
| HDSG | |
| (SEQ ID NO: 35) | |
| HDS | |
| (SEQ ID NO: 36) | |
| DSGY | |
| (SEQ ID NO: 37) | |
| DSG | |
| (SEQ ID NO: 38) | |
| SGY | |
| (SEQ ID NO: 1002) | |
| VHHQKLVFFA | |
| (SEQ ID NO: 1003) | |
| VHHQKLVFF | |
| (SEQ ID NO: 1004) | |
| VHHQKLVF | |
| (SEQ ID NO: 1005) | |
| VHHQKLV | |
| (SEQ ID NO: 1006) | |
| VHHQKL | |
| (SEQ ID NO: 1007) | |
| HHQKLVFFAE | |
| (SEQ ID NO: 1008) | |
| HHQKLVFFA | |
| (SEQ ID NO: 1009) | |
| HHQKLVFF | |
| (SEQ ID NO: 1010) | |
| HHQKLVF | |
| (SEQ ID NO: 1011) | |
| HHQKLV | |
| (SEQ ID NO: 1012) | |
| HHQKL | |
| (SEQ ID NO: 1013) | |
| HQKLVFFAED | |
| (SEQ ID NO: 1014) | |
| HQKLVFFAE | |
| (SEQ ID NO: 1015) | |
| HQKLVFFA | |
| (SEQ ID NO: 1016) | |
| HQKLVFF | |
| (SEQ ID NO: 1017) | |
| HQKLVF | |
| (SEQ ID NO: 1018) | |
| HQKLV | |
| (SEQ ID NO: 1019) | |
| HQKL | |
| (SEQ ID NO: 1020) | |
| QKLVFFAEDV | |
| (SEQ ID NO: 1021) | |
| QKLVFFAED | |
| (SEQ ID NO: 1022) | |
| QKLVFFAE | |
| (SEQ ID NO: 1023) | |
| QKLVFFA | |
| (SEQ ID NO: 1024) | |
| QKLVFF | |
| (SEQ ID NO: 1025) | |
| QKLVF | |
| (SEQ ID NO: 1026) | |
| QKLV | |
| (SEQ ID NO: 1027) | |
| QKL | |
| (SEQ ID NO: 1028) | |
| KLVFFAEDVG | |
| (SEQ ID NO: 1029) | |
| KLVFFAEDV | |
| (SEQ ID NO: 1030) | |
| KLVFFAED | |
| (SEQ ID NO: 1031) | |
| KLVFFAE | |
| (SEQ ID NO: 1032) | |
| KLVFFA | |
| (SEQ ID NO: 1033) | |
| KLVFF | |
| (SEQ ID NO: 1034) | |
| KLVF | |
| (SEQ ID NO: 1035) | |
| KLV | |
| (SEQ ID NO: 1036) | |
| LVFFAEDVG | |
| (SEQ ID NO: 1037) | |
| LVFFAEDV | |
| (SEQ ID NO: 1038) | |
| LVFFAED | |
| (SEQ ID NO: 1039) | |
| LVFFAE | |
| (SEQ ID NO: 1040) | |
| LVFFA | |
| (SEQ ID NO: 1041) | |
| LVFF | |
| (SEQ ID NO: 1042) | |
| LVF | |
| (SEQ ID NO: 1043) | |
| VFFAEDVG | |
| (SEQ ID NO: 1044) | |
| VFFAEDV | |
| (SEQ ID NO: 1045) | |
| VFFAED | |
| (SEQ ID NO: 1046) | |
| VFFAE | |
| (SEQ ID NO: 1047) | |
| VFFA | |
| (SEQ ID NO: 1048) | |
| VFF | |
| (SEQ ID NO: 1049) | |
| FFAEDVG | |
| (SEQ ID NO: 1050) | |
| FFAEDV | |
| (SEQ ID NO: 1051) | |
| FFAED | |
| (SEQ ID NO: 1052) | |
| FFAE | |
| (SEQ ID NO: 1053) | |
| FFA | |
| (SEQ ID NO: 1054) | |
| FAEDVG | |
| (SEQ ID NO: 1055) | |
| FAEDV | |
| (SEQ ID NO: 1056) | |
| FAED | |
| (SEQ ID NO: 1057) | |
| FAE | |
| Tau immunogens: | |
| (SEQ ID NO: 39) | |
| QIVYKPV | |
| (SEQ ID NO: 40) | |
| QIVYKP | |
| (SEQ ID NO: 41) | |
| QIVYKSV | |
| (SEQ ID NO: 42) | |
| EIVYKSV | |
| (SEQ ID NO: 997) | |
| QIVYKS | |
| (SEQ ID NO: 43) | |
| EIVYKSP | |
| (SEQ ID NO: 998) | |
| EIVYKS | |
| (SEQ ID NO: 44) | |
| EIVYKPV | |
| (SEQ ID NO: 999) | |
| EIVYKP | |
| (SEQ ID NO: 45) | |
| IVYKSPV | |
| (SEQ ID NO: 46) | |
| IVYK | |
| (SEQ ID NO: 1000) | |
| CNIKHVPG | |
| (SEQ ID NO: 47) | |
| CNIKHVP | |
| (SEQ ID NO: 48) | |
| NIKHVP | |
| (SEQ ID NO: 49) | |
| HVPGGG | |
| (SEQ ID NO: 50) | |
| HVPGG | |
| (SEQ ID NO: 51) | |
| HKPGGG | |
| (SEQ ID NO: 52) | |
| HKPGG | |
| (SEQ ID NO: 53) | |
| KHVPGGG | |
| (SEQ ID NO: 54) | |
| KHVPGG | |
| (SEQ ID NO: 55) | |
| HQPGGG | |
| (SEQ ID NO: 56) | |
| HQPGG | |
| (SEQ ID NO: 57) | |
| NIKHVPG | |
| (SEQ ID NO: 146) | |
| VQIINK | |
| (SEQ ID NO: 147) | |
| VQIINKK | |
| (SEQ ID NO: 148) | |
| VQIINKKL | |
| (SEQ ID NO: 149) | |
| QIINK | |
| (SEQ ID NO: 150) | |
| QIINKK | |
| (SEQ ID NO: 151) | |
| QIINKKL | |
| (SEQ ID NO: 152) | |
| EAAGHVTQC | |
| (SEQ ID NO: 153) | |
| EAAGHVTQAR | |
| (SEQ ID NO: 154) | |
| AAGHVTQAC | |
| (SEQ ID NO: 155) | |
| AGHVTQARC | |
| (SEQ ID NO: 156) | |
| AGHVTQAR | |
| (SEQ ID NO: 157) | |
| GYTMHQD | |
| (SEQ ID NO: 158) | |
| QGGYTMHC | |
| (SEQ ID NO: 159) | |
| QGGYTMHQD | |
| (SEQ ID NO: 160) | |
| GGYTMHQC | |
| (SEQ ID NO: 161) | |
| VPGGGSVQIV | |
| (SEQ ID NO: 162) | |
| PGGGSVQIV | |
| (SEQ ID NO: 163) | |
| GGGSVQIV | |
| (SEQ ID NO: 164) | |
| GGSVQIV | |
| (SEQ ID NO: 165) | |
| GSVQIV | |
| (SEQ ID NO: 166) | |
| SVQIV | |
| (SEQ ID NO: 167) | |
| VQIV | |
| (SEQ ID NO: 168) | |
| QIV | |
| (SEQ ID NO: 169) | |
| PGGGSVQIVY | |
| (SEQ ID NO: 170) | |
| GGGSVQIVY | |
| (SEQ ID NO: 171) | |
| GGSVQIVY | |
| (SEQ ID NO: 172) | |
| GSVQIVY | |
| (SEQ ID NO: 173) | |
| SVQIVY | |
| (SEQ ID NO: 174) | |
| VQIVY | |
| (SEQ ID NO: 175) | |
| QIVY | |
| (SEQ ID NO: 176) | |
| IVY | |
| (SEQ ID NO: 177) | |
| GGGSVQIVYK | |
| (SEQ ID NO: 178) | |
| GGSVQIVYK | |
| (SEQ ID NO: 179) | |
| GSVQIVYK | |
| (SEQ ID NO: 180) | |
| SVQIVYK | |
| (SEQ ID NO: 181) | |
| VQIVYK | |
| (SEQ ID NO: 182) | |
| QIVYK | |
| (SEQ ID NO: 183) | |
| VYK | |
| (SEQ ID NO: 184) | |
| GGSVQIVYKP | |
| (SEQ ID NO: 185) | |
| GSVQIVYKP | |
| (SEQ ID NO: 186) | |
| SVQIVYKP | |
| (SEQ ID NO: 187) | |
| VQIVYKP | |
| (SEQ ID NO: 188) | |
| IVYKP | |
| (SEQ ID NO: 189) | |
| VYKP | |
| (SEQ ID NO: 190) | |
| YKP | |
| (SEQ ID NO: 191) | |
| GSVQIVYKPV | |
| (SEQ ID NO: 192) | |
| SVQIVYKPV | |
| (SEQ ID NO: 193) | |
| VQIVYKPV | |
| (SEQ ID NO: 194) | |
| IVYKPV | |
| (SEQ ID NO: 195) | |
| VYKPV | |
| (SEQ ID NO: 196) | |
| YKPV | |
| (SEQ ID NO: 197) | |
| KPV | |
| (SEQ ID NO: 198) | |
| SVQIVYKPVD | |
| (SEQ ID NO: 199) | |
| VQIVYKPVD | |
| (SEQ ID NO: 200) | |
| QIVYKPVD | |
| (SEQ ID NO: 201) | |
| IVYKPVD | |
| (SEQ ID NO: 203) | |
| VYKPVD | |
| (SEQ ID NO: 204) | |
| YKPVD | |
| (SEQ ID NO: 205) | |
| KPVD | |
| (SEQ ID NO: 206) | |
| PVD | |
| (SEQ ID NO: 207) | |
| VQIVYKPVDL | |
| (SEQ ID NO: 208) | |
| QIVYKPVDL | |
| (SEQ ID NO: 209) | |
| IVYKPVDL | |
| (SEQ ID NO: 210) | |
| VYKPVDL | |
| (SEQ ID NO: 211) | |
| YKPVDL | |
| (SEQ ID NO: 212) | |
| KPVDL | |
| (SEQ ID NO: 213) | |
| PVDL | |
| (SEQ ID NO: 214) | |
| VDL | |
| (SEQ ID NO: 215) | |
| QIVYKPVDLS | |
| (SEQ ID NO: 216) | |
| IVYKPVDLS | |
| (SEQ ID NO: 217) | |
| VYKPVDLS | |
| (SEQ ID NO: 218) | |
| YKPVDLS | |
| (SEQ ID NO: 219) | |
| KPVDLS | |
| (SEQ ID NO: 220) | |
| PVDLS | |
| (SEQ ID NO: 221) | |
| VDLS | |
| (SEQ ID NO: 222) | |
| DLS | |
| (SEQ ID NO: 223) | |
| IVYKPVDLSK | |
| (SEQ ID NO: 224) | |
| VYKPVDLSK | |
| (SEQ ID NO: 225) | |
| YKPVDLSK | |
| (SEQ ID NO: 226) | |
| KPVDLSK | |
| (SEQ ID NO: 227) | |
| PVDLSK | |
| (SEQ ID NO: 228) | |
| VDLSK | |
| (SEQ ID NO: 229) | |
| DLSK | |
| (SEQ ID NO: 230) | |
| LSK | |
| (SEQ ID NO: 231) | |
| VYKPVDLSKV | |
| (SEQ ID NO: 232) | |
| YKPVDLSKV | |
| (SEQ ID NO: 233) | |
| KPVDLSKV | |
| (SEQ ID NO: 234) | |
| PVDLSKV | |
| (SEQ ID NO: 235) | |
| VDLSKV | |
| (SEQ ID NO: 236) | |
| DLSKV | |
| (SEQ ID NO: 237) | |
| LSKV | |
| (SEQ ID NO: 238) | |
| SKV | |
| (SEQ ID NO: 239) | |
| YKPVDLSKVT | |
| (SEQ ID NO: 240) | |
| KPVDLSKVT | |
| (SEQ ID NO: 241) | |
| PVDLSKVT | |
| (SEQ ID NO: 242) | |
| VDLSKVT | |
| (SEQ ID NO: 243) | |
| AKTDHGAEIV | |
| (SEQ ID NO: 244) | |
| KTDHGAEIV | |
| (SEQ ID NO: 245) | |
| TDHGAEIV | |
| (SEQ ID NO: 246) | |
| DHGAEIV | |
| (SEQ ID NO: 247) | |
| HGAEIV | |
| (SEQ ID NO: 248) | |
| GAEIV | |
| (SEQ ID NO: 249) | |
| AEIV | |
| (SEQ ID NO: 250) | |
| EIV | |
| (SEQ ID NO: 251) | |
| KTDHGAEIVY | |
| (SEQ ID NO: 252) | |
| TDHGAEIVY | |
| (SEQ ID NO: 253) | |
| DHGAEIVY | |
| (SEQ ID NO: 254) | |
| HGAEIVY | |
| (SEQ ID NO: 255) | |
| GAEIVY | |
| (SEQ ID NO: 256) | |
| AEIVY | |
| (SEQ ID NO: 257) | |
| EIVY | |
| (SEQ ID NO: 258) | |
| IVY | |
| (SEQ ID NO: 259) | |
| TDHGAEIVYK | |
| (SEQ ID NO: 260) | |
| DHGAEIVYK | |
| (SEQ ID NO: 261) | |
| HGAEIVYK | |
| (SEQ ID NO: 262) | |
| GAEIVYK | |
| (SEQ ID NO: 263) | |
| AEIVYK | |
| (SEQ ID NO: 264) | |
| EIVYK | |
| (SEQ ID NO: 265) | |
| IVYK | |
| (SEQ ID NO: 266) | |
| DHGAEIVYKS | |
| (SEQ ID NO: 267) | |
| HGAEIVYKS | |
| (SEQ ID NO: 268) | |
| GAEIVYKS | |
| (SEQ ID NO: 269) | |
| AEIVYKS | |
| (SEQ ID NO: 270) | |
| EIVYKS | |
| (SEQ ID NO: 271) | |
| IVYKS | |
| (SEQ ID NO: 272) | |
| VYKS | |
| (SEQ ID NO: 273) | |
| YKS | |
| (SEQ ID NO: 274) | |
| HGAEIVYKSP | |
| (SEQ ID NO: 275) | |
| GAEIVYKSP | |
| (SEQ ID NO: 276) | |
| AEIVYKSP | |
| (SEQ ID NO: 277) | |
| EIVYKSP | |
| (SEQ ID NO: 278) | |
| IVYKSP | |
| (SEQ ID NO: 279) | |
| VYKSP | |
| (SEQ ID NO: 280) | |
| YKSP | |
| (SEQ ID NO: 281) | |
| KSP | |
| (SEQ ID NO: 282) | |
| GAEIVYKSPV | |
| (SEQ ID NO: 283) | |
| AEIVYKSPV | |
| (SEQ ID NO: 284) | |
| EIVYKSPV | |
| (SEQ ID NO: 285) | |
| IVYKSPV | |
| (SEQ ID NO: 286) | |
| VYKSPV | |
| (SEQ ID NO: 287) | |
| YKSPV | |
| (SEQ ID NO: 288) | |
| KSPV | |
| (SEQ ID NO: 289) | |
| SPV | |
| (SEQ ID NO: 290) | |
| AEIVYKSPVV | |
| (SEQ ID NO: 291) | |
| EIVYKSPVV | |
| (SEQ ID NO: 292) | |
| IVYKSPVV | |
| (SEQ ID NO: 293) | |
| VYKSPVV | |
| (SEQ ID NO: 294) | |
| YKSPVV | |
| (SEQ ID NO: 295) | |
| KSPVV | |
| (SEQ ID NO: 296) | |
| SPVV | |
| (SEQ ID NO: 297) | |
| PVV | |
| (SEQ ID NO: 298) | |
| EIVYKSPVVS | |
| (SEQ ID NO: 299) | |
| IVYKSPVVS | |
| (SEQ ID NO: 300) | |
| VYKSPVVS | |
| (SEQ ID NO: 301) | |
| YKSPVVS | |
| (SEQ ID NO: 302) | |
| KSPVVS | |
| (SEQ ID NO: 303) | |
| SPVVS | |
| (SEQ ID NO: 304) | |
| PVVS | |
| (SEQ ID NO: 305) | |
| VVS | |
| (SEQ ID NO: 306) | |
| IVYKSPVVSG | |
| (SEQ ID NO: 307) | |
| VYKSPVVSG | |
| (SEQ ID NO: 308) | |
| YKSPVVSG | |
| (SEQ ID NO: 309) | |
| KSPVVSG | |
| (SEQ ID NO: 310) | |
| SPVVSG | |
| (SEQ ID NO: 311) | |
| PVVSG | |
| (SEQ ID NO: 312) | |
| VVSG | |
| (SEQ ID NO: 313) | |
| VSG | |
| (SEQ ID NO: 314) | |
| VYKSPVVSGD | |
| (SEQ ID NO: 315) | |
| YKSPVVSGD | |
| (SEQ ID NO: 316) | |
| KSPVVSGD | |
| (SEQ ID NO: 317) | |
| SPVVSGD | |
| (SEQ ID NO: 318) | |
| PVVSGD | |
| (SEQ ID NO: 319) | |
| VVSGD | |
| (SEQ ID NO: 320) | |
| VSGD | |
| (SEQ ID NO: 321) | |
| SGD | |
| (SEQ ID NO: 322) | |
| YKSPVVSGDT | |
| (SEQ ID NO: 323) | |
| KSPVVSGDT | |
| (SEQ ID NO: 324) | |
| SPVVSGDT | |
| (SEQ ID NO: 325) | |
| PVVSGDT | |
| (SEQ ID NO: 326) | |
| VVSGDT | |
| (SEQ ID NO: 327) | |
| VSGDT | |
| (SEQ ID NO: 328) | |
| SGDT | |
| (SEQ ID NO: 329) | |
| GDT | |
| (SEQ ID NO: 330) | |
| KSPVVSGDTS | |
| (SEQ ID NO: 331) | |
| SPVVSGDTS | |
| (SEQ ID NO: 332) | |
| PVVSGDTS | |
| (SEQ ID NO: 333) | |
| VVSGDTS | |
| (SEQ ID NO: 334) | |
| VSGDTS | |
| (SEQ ID NO: 335) | |
| SGDTS | |
| (SEQ ID NO: 336) | |
| GDTS | |
| (SEQ ID NO: 337) | |
| DTS | |
| (SEQ ID NO: 338) | |
| SPVVSGDTSP | |
| (SEQ ID NO: 339) | |
| PVVSGDTSP | |
| (SEQ ID NO: 340) | |
| VVSGDTSP | |
| (SEQ ID NO: 341) | |
| VSGDTSP | |
| (SEQ ID NO: 342) | |
| SGDTSP | |
| (SEQ ID NO: 343) | |
| GDTSP | |
| (SEQ ID NO: 344) | |
| DTSP | |
| (SEQ ID NO: 345) | |
| TSP | |
| (SEQ ID NO: 346) | |
| PVVSGDTSPR | |
| (SEQ ID NO: 347) | |
| VVSGDTSPR | |
| (SEQ ID NO: 348) | |
| VSGDTSPR | |
| (SEQ ID NO: 349) | |
| SGDTSPR | |
| (SEQ ID NO: 350) | |
| GDTSPR | |
| (SEQ ID NO: 351) | |
| DTSPR | |
| (SEQ ID NO: 352) | |
| TSPR | |
| (SEQ ID NO: 353) | |
| SPR | |
| (SEQ ID NO: 354) | |
| HQPGGGKVQI | |
| (SEQ ID NO: 355) | |
| QPGGGKVQI | |
| (SEQ ID NO: 356) | |
| PGGGKVQI | |
| (SEQ ID NO: 357) | |
| GGGKVQI | |
| (SEQ ID NO: 358) | |
| GGKVQI | |
| (SEQ ID NO: 359) | |
| GKVQI | |
| (SEQ ID NO: 360) | |
| KVQI | |
| (SEQ ID NO: 361) | |
| VQI | |
| (SEQ ID NO: 362) | |
| QPGGGKVQII | |
| (SEQ ID NO: 363) | |
| PGGGKVQII | |
| (SEQ ID NO: 365) | |
| GGGKVQII | |
| (SEQ ID NO: 366) | |
| GGKVQII | |
| (SEQ ID NO: 367) | |
| GKVQII | |
| (SEQ ID NO: 368) | |
| KVQII | |
| (SEQ ID NO: 369) | |
| VQII | |
| (SEQ ID NO: 370) | |
| QII | |
| (SEQ ID NO: 371) | |
| PGGGKVQIIN | |
| (SEQ ID NO: 372) | |
| GGGKVQIIN | |
| (SEQ ID NO: 373) | |
| GGKVQIIN | |
| (SEQ ID NO: 374) | |
| GKVQIIN | |
| (SEQ ID NO: 375) | |
| KVQIIN | |
| (SEQ ID NO: 376) | |
| VQIIN | |
| (SEQ ID NO: 377) | |
| QIIN | |
| (SEQ ID NO: 378) | |
| IIN | |
| (SEQ ID NO: 379) | |
| GGGKVQIINK | |
| (SEQ ID NO: 380) | |
| GGKVQIINK | |
| (SEQ ID NO: 381) | |
| GKVQIINK | |
| (SEQ ID NO: 382) | |
| KVQIINK | |
| (SEQ ID NO: 383) | |
| IINK | |
| (SEQ ID NO: 384) | |
| INK | |
| (SEQ ID NO: 385) | |
| GGKVQIINKK | |
| (SEQ ID NO: 386) | |
| GKVQIINKK | |
| (SEQ ID NO: 387) | |
| KVQIINKK | |
| (SEQ ID NO: 388) | |
| IINKK | |
| (SEQ ID NO: 389) | |
| INKK | |
| (SEQ ID NO: 390) | |
| NKK | |
| (SEQ ID NO: 391) | |
| GKVQIINKKL | |
| (SEQ ID NO: 392) | |
| KVQIINKKL | |
| (SEQ ID NO: 393) | |
| IINKKL | |
| (SEQ ID NO: 394) | |
| INKKL | |
| (SEQ ID NO: 395) | |
| NKKL | |
| (SEQ ID NO: 396) | |
| KKL | |
| (SEQ ID NO: 397) | |
| KVQIINKKLD | |
| (SEQ ID NO: 398) | |
| VQIINKKLD | |
| (SEQ ID NO: 399) | |
| QIINKKLD | |
| (SEQ ID NO: 400) | |
| IINKKLD | |
| (SEQ ID NO: 401) | |
| INKKLD | |
| (SEQ ID NO: 402) | |
| NKKLD | |
| (SEQ ID NO: 403) | |
| KKLD | |
| (SEQ ID NO: 404) | |
| KLD | |
| (SEQ ID NO: 405) | |
| VQIINKKLDL | |
| (SEQ ID NO: 406) | |
| QIINKKLDL | |
| (SEQ ID NO: 407) | |
| IINKKLDL | |
| (SEQ ID NO: 408) | |
| INKKLDL | |
| (SEQ ID NO: 409) | |
| NKKLDL | |
| (SEQ ID NO: 410) | |
| KKLDL | |
| (SEQ ID NO: 411) | |
| KLDL | |
| (SEQ ID NO: 412) | |
| LDL | |
| (SEQ ID NO: 413) | |
| QIINKKLDLS | |
| (SEQ ID NO: 414) | |
| IINKKLDLS | |
| (SEQ ID NO: 415) | |
| INKKLDLS | |
| (SEQ ID NO: 416) | |
| NKKLDLS | |
| (SEQ ID NO: 417) | |
| KKLDLS | |
| (SEQ ID NO: 418) | |
| KLDLS | |
| (SEQ ID NO: 419) | |
| LDLS | |
| (SEQ ID NO: 420) | |
| IINKKLDLSN | |
| (SEQ ID NO: 421) | |
| INKKLDLSN | |
| (SEQ ID NO: 422) | |
| NKKLDLSN | |
| (SEQ ID NO: 423) | |
| KKLDLSN | |
| (SEQ ID NO: 424) | |
| KLDLSN | |
| (SEQ ID NO: 425) | |
| LDLSN | |
| (SEQ ID NO: 426) | |
| DLSN | |
| (SEQ ID NO: 427) | |
| LSN | |
| (SEQ ID NO: 428) | |
| INKKLDLSNV | |
| (SEQ ID NO: 429) | |
| NKKLDLSNV | |
| (SEQ ID NO: 430) | |
| KKLDLSNV | |
| (SEQ ID NO: 431) | |
| KLDLSNV | |
| (SEQ ID NO: 432) | |
| LDLSNV | |
| (SEQ ID NO: 433) | |
| DLSNV | |
| (SEQ ID NO: 434) | |
| LSNV | |
| (SEQ ID NO: 435) | |
| SNV | |
| (SEQ ID NO: 436) | |
| NKKLDLSNVQ | |
| (SEQ ID NO: 437) | |
| KKLDLSNVQ | |
| (SEQ ID NO: 438) | |
| KLDLSNVQ | |
| (SEQ ID NO: 439) | |
| LDLSNVQ | |
| (SEQ ID NO: 440) | |
| DLSNVQ | |
| (SEQ ID NO: 441) | |
| LSNVQ | |
| (SEQ ID NO: 442) | |
| SNVQ | |
| (SEQ ID NO: 443) | |
| NVQ | |
| (SEQ ID NO: 444) | |
| KKLDLSNVQS | |
| (SEQ ID NO: 445) | |
| KLDLSNVQS | |
| (SEQ ID NO: 446) | |
| LDLSNVQS | |
| (SEQ ID NO: 447) | |
| DLSNVQS | |
| (SEQ ID NO: 448) | |
| LSNVQS | |
| (SEQ ID NO: 449) | |
| SNVQS | |
| (SEQ ID NO: 450) | |
| NVQS | |
| (SEQ ID NO: 451) | |
| VQS | |
| (SEQ ID NO: 452) | |
| SKCGSKDNIK | |
| (SEQ ID NO: 453) | |
| KCGSKDNIK | |
| (SEQ ID NO: 454) | |
| CGSKDNIK | |
| (SEQ ID NO: 455) | |
| GSKDNIK | |
| (SEQ ID NO: 456) | |
| SKDNIK | |
| (SEQ ID NO: 457) | |
| KDNIK | |
| (SEQ ID NO: 458) | |
| DNIK | |
| (SEQ ID NO: 459) | |
| NIK | |
| (SEQ ID NO: 460) | |
| KCGSKDNIKH | |
| (SEQ ID NO: 461) | |
| CGSKDNIKH | |
| (SEQ ID NO: 462) | |
| GSKDNIKH | |
| (SEQ ID NO: 463) | |
| SKDNIKH | |
| (SEQ ID NO: 464) | |
| KDNIKH | |
| (SEQ ID NO: 465) | |
| DNIKH | |
| (SEQ ID NO: 466) | |
| NIKH | |
| (SEQ ID NO: 467) | |
| IKH | |
| (SEQ ID NO: 468) | |
| CGSKDNIKHV | |
| (SEQ ID NO: 469) | |
| GSKDNIKHV | |
| (SEQ ID NO: 470) | |
| SKDNIKHV | |
| (SEQ ID NO: 471) | |
| KDNIKHV | |
| (SEQ ID NO: 472) | |
| DNIKHV | |
| (SEQ ID NO: 473) | |
| NIKHV | |
| (SEQ ID NO: 474) | |
| IKHV | |
| (SEQ ID NO: 475) | |
| KHV | |
| (SEQ ID NO: 476) | |
| GSKDNIKHVP | |
| (SEQ ID NO: 477) | |
| SKDNIKHVP | |
| (SEQ ID NO: 478) | |
| KDNIKHVP | |
| (SEQ ID NO: 479) | |
| DNIKHVP | |
| (SEQ ID NO: 480) | |
| IKHVP | |
| (SEQ ID NO: 481) | |
| KHVP | |
| (SEQ ID NO: 482) | |
| HVP | |
| (SEQ ID NO: 483) | |
| SKDNIKHVPG | |
| (SEQ ID NO: 484) | |
| KDNIKHVPG | |
| (SEQ ID NO: 485) | |
| DNIKHVPG | |
| (SEQ ID NO: 486) | |
| NIKHVPG | |
| (SEQ ID NO: 487) | |
| IKHVPG | |
| (SEQ ID NO: 488) | |
| KHVPG | |
| (SEQ ID NO: 489) | |
| HVPG | |
| (SEQ ID NO: 490) | |
| VPG | |
| (SEQ ID NO: 491) | |
| KDNIKHVPGG | |
| (SEQ ID NO: 492) | |
| DNIKHVPGG | |
| (SEQ ID NO: 493) | |
| NIKHVPGG | |
| (SEQ ID NO: 494) | |
| IKHVPGG | |
| (SEQ ID NO: 495) | |
| KHVPGG | |
| (SEQ ID NO: 496) | |
| VPGG | |
| (SEQ ID NO: 497) | |
| PGG | |
| (SEQ ID NO: 498) | |
| DNIKHVPGGG | |
| (SEQ ID NO: 499) | |
| NIKHVPGGG | |
| (SEQ ID NO: 500) | |
| IKHVPGGG | |
| (SEQ ID NO: 501) | |
| VPGGG | |
| (SEQ ID NO: 502) | |
| PGGG | |
| (SEQ ID NO: 503) | |
| GGG | |
| (SEQ ID NO: 504) | |
| NIKHVPGGGS | |
| (SEQ ID NO: 505) | |
| IKHVPGGGS | |
| (SEQ ID NO: 506) | |
| KHVPGGGS | |
| (SEQ ID NO: 507) | |
| HVPGGGS | |
| (SEQ ID NO: 508) | |
| VPGGGS | |
| (SEQ ID NO: 509) | |
| PGGGS | |
| (SEQ ID NO: 510) | |
| GGGS | |
| (SEQ ID NO: 511) | |
| GGS | |
| (SEQ ID NO: 512) | |
| IKHVPGGGSV | |
| (SEQ ID NO: 513) | |
| KHVPGGGSV | |
| (SEQ ID NO: 514) | |
| HVPGGGSV | |
| (SEQ ID NO: 515) | |
| VPGGGSV | |
| (SEQ ID NO: 516) | |
| PGGGSV | |
| (SEQ ID NO: 517) | |
| GGGSV | |
| (SEQ ID NO: 518) | |
| GGSV | |
| (SEQ ID NO: 519) | |
| GSV | |
| (SEQ ID NO: 520) | |
| KHVPGGGSVQ | |
| (SEQ ID NO: 521) | |
| HVPGGGSVQ | |
| (SEQ ID NO: 522) | |
| VPGGGSVQ | |
| (SEQ ID NO: 523) | |
| PGGGSVQ | |
| (SEQ ID NO: 524) | |
| GGGSVQ | |
| (SEQ ID NO: 525) | |
| GGSVQ | |
| (SEQ ID NO: 526) | |
| GSVQ | |
| (SEQ ID NO: 527) | |
| SVQ | |
| (SEQ ID NO: 528) | |
| HVPGGGSVQI | |
| (SEQ ID NO: 529) | |
| VPGGGSVQI | |
| (SEQ ID NO: 530) | |
| PGGGSVQI | |
| (SEQ ID NO: 531) | |
| GGGSVQI | |
| (SEQ ID NO: 532) | |
| GGSVQI | |
| (SEQ ID NO: 533) | |
| GSVQI | |
| (SEQ ID NO: 534) | |
| SVQI | |
| (SEQ ID NO: 535) | |
| GGSVQIVYKS | |
| (SEQ ID NO: 536) | |
| GSVQIVYKS | |
| (SEQ ID NO: 537) | |
| SVQIVYKS | |
| (SEQ ID NO: 538) | |
| VQIVYKS | |
| (SEQ ID NO: 539) | |
| QIVYKS | |
| (SEQ ID NO: 540) | |
| IVYKS | |
| (SEQ ID NO: 541) | |
| VYKS | |
| (SEQ ID NO: 542) | |
| YKS | |
| (SEQ ID NO: 543) | |
| GSVQIVYKSV | |
| (SEQ ID NO: 544) | |
| SVQIVYKSV | |
| (SEQ ID NO: 545) | |
| VQIVYKSV | |
| (SEQ ID NO: 546) | |
| QIVYKSV | |
| (SEQ ID NO: 547) | |
| IVYKSV | |
| (SEQ ID NO: 548) | |
| VYKSV | |
| (SEQ ID NO: 549) | |
| YKSV | |
| (SEQ ID NO: 550) | |
| SVQIVYKSVD | |
| (SEQ ID NO: 551) | |
| VQIVYKSVD | |
| (SEQ ID NO: 552) | |
| QIVYKSVD | |
| (SEQ ID NO: 553) | |
| IVYKSVD | |
| (SEQ ID NO: 554) | |
| VYKSVD | |
| (SEQ ID NO: 555) | |
| YKSVD | |
| (SEQ ID NO: 556) | |
| KSVD | |
| (SEQ ID NO: 557) | |
| SVD | |
| (SEQ ID NO: 558) | |
| VQIVYKSVDL | |
| (SEQ ID NO: 559) | |
| QIVYKSVDL | |
| (SEQ ID NO: 560) | |
| IVYKSVDL | |
| (SEQ ID NO: 561) | |
| VYKSVDL | |
| (SEQ ID NO: 562) | |
| YKSVDL | |
| (SEQ ID NO: 563) | |
| KSVDL | |
| (SEQ ID NO: 564) | |
| SVDL | |
| (SEQ ID NO: 565) | |
| QIVYKSVDLS | |
| (SEQ ID NO: 566) | |
| IVYKSVDLS | |
| (SEQ ID NO: 567) | |
| VYKSVDLS | |
| (SEQ ID NO: 568) | |
| YKSVDLS | |
| (SEQ ID NO: 569) | |
| KSVDLS | |
| (SEQ ID NO: 570) | |
| SVDLS | |
| (SEQ ID NO: 571) | |
| IVYKSVDLSK | |
| (SEQ ID NO: 572) | |
| VYKSVDLSK | |
| (SEQ ID NO: 573) | |
| YKSVDLSK | |
| (SEQ ID NO: 574) | |
| KSVDLSK | |
| (SEQ ID NO: 364) | |
| SVDLSK | |
| (SEQ ID NO: 575) | |
| VYKSVDLSKV | |
| (SEQ ID NO: 576) | |
| YKSVDLSKV | |
| (SEQ ID NO: 577) | |
| KSVDLSKV | |
| (SEQ ID NO: 578) | |
| SVDLSKV | |
| (SEQ ID NO: 579) | |
| YKSVDLSKVT | |
| (SEQ ID NO: 580) | |
| KSVDLSKVT | |
| (SEQ ID NO: 581) | |
| SVDLSKVT | |
| (SEQ ID NO: 582) | |
| DLSKVT | |
| (SEQ ID NO: 583) | |
| LSKVT | |
| (SEQ ID NO: 584) | |
| SKVT | |
| (SEQ ID NO: 585) | |
| KVT | |
| (SEQ ID NO: 586) | |
| HGAEIVYKSV | |
| (SEQ ID NO: 587) | |
| GAEIVYKSV | |
| (SEQ ID NO: 588) | |
| AEIVYKSV | |
| (SEQ ID NO: 589) | |
| GAEIVYKSVV | |
| (SEQ ID NO: 590) | |
| AEIVYKSVV | |
| (SEQ ID NO: 591) | |
| EIVYKSVV | |
| (SEQ ID NO: 592) | |
| IVYKSVV | |
| (SEQ ID NO: 593) | |
| VYKSVV | |
| (SEQ ID NO: 594) | |
| YKSVV | |
| (SEQ ID NO: 595) | |
| KSVV | |
| (SEQ ID NO: 596) | |
| SVV | |
| (SEQ ID NO: 597) | |
| AEIVYKSVVS | |
| (SEQ ID NO: 598) | |
| EIVYKSVVS | |
| (SEQ ID NO: 599) | |
| IVYKSVVS | |
| (SEQ ID NO: 600) | |
| VYKSVVS | |
| (SEQ ID NO: 601) | |
| YKSVVS | |
| (SEQ ID NO: 602) | |
| KSVVS | |
| (SEQ ID NO: 603) | |
| SVVS | |
| (SEQ ID NO: 604) | |
| EIVYKSVVSG | |
| (SEQ ID NO: 605) | |
| IVYKSVVSG | |
| (SEQ ID NO: 606) | |
| VYKSVVSG | |
| (SEQ ID NO: 607) | |
| YKSVVSG | |
| (SEQ ID NO: 608) | |
| KSVVSG | |
| (SEQ ID NO: 609) | |
| SVVSG | |
| (SEQ ID NO: 610) | |
| VVSG | |
| (SEQ ID NO: 611) | |
| VSG | |
| (SEQ ID NO: 612) | |
| IVYKSVVSGD | |
| (SEQ ID NO: 613) | |
| VYKSVVSGD | |
| (SEQ ID NO: 614) | |
| YKSVVSGD | |
| (SEQ ID NO: 615) | |
| KSVVSGD | |
| (SEQ ID NO: 616) | |
| SVVSGD | |
| (SEQ ID NO: 617) | |
| VVSGD | |
| (SEQ ID NO: 618) | |
| VYKSVVSGDT | |
| (SEQ ID NO: 619) | |
| YKSVVSGDT | |
| (SEQ ID NO: 620) | |
| KSVVSGDT | |
| (SEQ ID NO: 621) | |
| SVVSGDT | |
| (SEQ ID NO: 622) | |
| YKSVVSGDTS | |
| (SEQ ID NO: 623) | |
| KSVVSGDTS | |
| (SEQ ID NO: 624) | |
| SVVSGDTS | |
| (SEQ ID NO: 625) | |
| YKSVVSGDTS | |
| (SEQ ID NO: 626) | |
| KSVVSGDTS | |
| (SEQ ID NO: 627) | |
| SVVSGDTS | |
| (SEQ ID NO: 628) | |
| VVSGDTS | |
| (SEQ ID NO: 629) | |
| KSVVSGDTSP | |
| (SEQ ID NO: 630) | |
| SVVSGDTSP | |
| (SEQ ID NO: 631) | |
| SVVSGDTSPR | |
| (SEQ ID NO: 632) | |
| DHGAEIVYKP | |
| (SEQ ID NO: 633) | |
| HGAEIVYKP | |
| (SEQ ID NO: 634) | |
| GAEIVYKP | |
| (SEQ ID NO: 635) | |
| AEIVYKP | |
| (SEQ ID NO: 636) | |
| HGAEIVYKPV | |
| (SEQ ID NO: 637) | |
| GAEIVYKPV | |
| (SEQ ID NO: 638) | |
| AEIVYKPV | |
| (SEQ ID NO: 639) | |
| GAEIVYKPVV | |
| (SEQ ID NO: 640) | |
| AEIVYKPVV | |
| (SEQ ID NO: 641) | |
| EIVYKPVV | |
| (SEQ ID NO: 642) | |
| IVYKPVV | |
| (SEQ ID NO: 643) | |
| VYKPVV | |
| (SEQ ID NO: 644) | |
| YKPVV | |
| (SEQ ID NO: 645) | |
| KPVV | |
| (SEQ ID NO: 646) | |
| AEIVYKPVVS | |
| (SEQ ID NO: 647) | |
| EIVYKPVVS | |
| (SEQ ID NO: 648) | |
| IVYKPVVS | |
| (SEQ ID NO: 649) | |
| VYKPVVS | |
| (SEQ ID NO: 650) | |
| YKPVVS | |
| (SEQ ID NO: 651) | |
| KPVVS | |
| (SEQ ID NO: 652) | |
| EIVYKPVVSG | |
| (SEQ ID NO: 653) | |
| IVYKPVVSG | |
| (SEQ ID NO: 654) | |
| VYKPVVSG | |
| (SEQ ID NO: 655) | |
| YKPVVSG | |
| (SEQ ID NO: 656) | |
| KPVVSG | |
| (SEQ ID NO: 657) | |
| IVYKPVVSGD | |
| (SEQ ID NO: 658) | |
| VYKPVVSGD | |
| (SEQ ID NO: 659) | |
| YKPVVSGD | |
| (SEQ ID NO: 660) | |
| KPVVSGD | |
| (SEQ ID NO: 661) | |
| VYKPVVSGDT | |
| (SEQ ID NO: 662) | |
| YKPVVSGDT | |
| (SEQ ID NO: 663) | |
| KPVVSGDT | |
| (SEQ ID NO: 664) | |
| YKPVVSGDTS | |
| (SEQ ID NO: 665) | |
| KPVVSGDTS | |
| (SEQ ID NO: 666) | |
| PVVSGDTS | |
| (SEQ ID NO: 667) | |
| VVSGDTS | |
| (SEQ ID NO: 668) | |
| KPVVSGDTSP | |
| (SEQ ID NO: 669) | |
| CNIK | |
| (SEQ ID NO: 670) | |
| CNIKH | |
| (SEQ ID NO: 671) | |
| CNIKHV | |
| (SEQ ID NO: 672) | |
| CNIKHVPGG | |
| (SEQ ID NO: 673) | |
| CNIKHVPGGG | |
| (SEQ ID NO: 674) | |
| CNIKHVPGGGS | |
| (SEQ ID NO: 675) | |
| ENLKHQPGGG | |
| (SEQ ID NO: 676) | |
| NLKHQPGGG | |
| (SEQ ID NO: 677) | |
| LKHQPGGG | |
| (SEQ ID NO: 678) | |
| KHQPGGG | |
| (SEQ ID NO: 679) | |
| HQPGGG | |
| (SEQ ID NO: 680) | |
| QPGGG | |
| (SEQ ID NO: 681) | |
| TENLKHQPGG | |
| (SEQ ID NO: 682) | |
| ENLKHQPGG | |
| (SEQ ID NO: 683) | |
| NLKHQPGG | |
| (SEQ ID NO: 684) | |
| LKHQPGG | |
| (SEQ ID NO: 685) | |
| KHQPGG | |
| (SEQ ID NO: 686) | |
| HQPGG | |
| (SEQ ID NO: 687) | |
| QPGG | |
| (SEQ ID NO: 688) | |
| TENLKHQPG | |
| (SEQ ID NO: 689) | |
| ENLKHQPG | |
| (SEQ ID NO: 690) | |
| NLKHQPG | |
| (SEQ ID NO: 691) | |
| LKHQPG | |
| (SEQ ID NO: 692) | |
| KHQPG | |
| (SEQ ID NO: 693) | |
| HQPG | |
| (SEQ ID NO: 694) | |
| QPG | |
| (SEQ ID NO: 695) | |
| TENLKHQP | |
| (SEQ ID NO: 696) | |
| ENLKHQP | |
| (SEQ ID NO: 697) | |
| NLKHQP | |
| (SEQ ID NO: 698) | |
| LKHQP | |
| (SEQ ID NO: 699) | |
| KHQP | |
| (SEQ ID NO: 700) | |
| HQP | |
| (SEQ ID NO: 701) | |
| TENLKHQ | |
| (SEQ ID NO: 702) | |
| ENLKHQ | |
| (SEQ ID NO: 703) | |
| NLKHQ | |
| (SEQ ID NO: 704) | |
| LKHQ | |
| (SEQ ID NO: 705) | |
| KHQ | |
| (SEQ ID NO: 706) | |
| TENLKH | |
| (SEQ ID NO: 707) | |
| ENLKH | |
| (SEQ ID NO: 708) | |
| NLKH | |
| (SEQ ID NO: 709) | |
| LKH | |
| (SEQ ID NO: 710) | |
| TENLK | |
| (SEQ ID NO: 711) | |
| ENLK | |
| (SEQ ID NO: 712) | |
| NLK | |
| (SEQ ID NO: 713) | |
| TENL | |
| (SEQ ID NO: 714) | |
| ENL | |
| (SEQ ID NO: 715) | |
| TEN | |
| (SEQ ID NO: 716) | |
| KDNIKHVPGGG | |
| (SEQ ID NO: 717) | |
| KDNI | |
| (SEQ ID NO: 718) | |
| KDN | |
| (SEQ ID NO: 719) | |
| IKHVGGG | |
| (SEQ ID NO: 720) | |
| IKHVGG | |
| (SEQ ID NO: 721) | |
| IKHVG | |
| (SEQ ID NO: 722) | |
| KHVGGG | |
| (SEQ ID NO: 723) | |
| KHVGG | |
| (SEQ ID NO: 724) | |
| KHVG | |
| (SEQ ID NO: 725) | |
| LGNIHHKPGGG | |
| (SEQ ID NO: 726) | |
| GNIHHKPGGG | |
| (SEQ ID NO: 727) | |
| NIHHKPGGG | |
| (SEQ ID NO: 728) | |
| IHHKPGGG | |
| (SEQ ID NO: 729) | |
| HHKPGGG | |
| (SEQ ID NO: 730) | |
| KPGGG | |
| (SEQ ID NO: 731) | |
| LGNIHHKPGG | |
| (SEQ ID NO: 732) | |
| GNIHHKPGG | |
| (SEQ ID NO: 733) | |
| NIHHKPGG | |
| (SEQ ID NO: 734) | |
| IHHKPGG | |
| (SEQ ID NO: 735) | |
| HHKPGG | |
| (SEQ ID NO: 736) | |
| KPGG | |
| (SEQ ID NO: 737) | |
| LGNIHHKPG | |
| (SEQ ID NO: 738) | |
| GNIHHKPG | |
| (SEQ ID NO: 739) | |
| NIHHKPG | |
| (SEQ ID NO: 740) | |
| IHHKPG | |
| (SEQ ID NO: 741) | |
| HHKPG | |
| (SEQ ID NO: 742) | |
| HKPG | |
| (SEQ ID NO: 743) | |
| KPG | |
| (SEQ ID NO: 744) | |
| LGNIHHKP | |
| (SEQ ID NO: 745) | |
| GNIHHKP | |
| (SEQ ID NO: 746) | |
| NIHHKP | |
| (SEQ ID NO: 747) | |
| IHHKP | |
| (SEQ ID NO: 748) | |
| HHKP | |
| (SEQ ID NO: 749) | |
| HKP | |
| (SEQ ID NO: 750) | |
| LGNIHHK | |
| (SEQ ID NO: 751) | |
| GNIHHK | |
| (SEQ ID NO: 752) | |
| NIHHK | |
| (SEQ ID NO: 753) | |
| IHHK | |
| (SEQ ID NO: 754) | |
| HHK | |
| (SEQ ID NO: 755) | |
| LGNIHH | |
| (SEQ ID NO: 756) | |
| GNIHH | |
| (SEQ ID NO: 757) | |
| NIHH | |
| (SEQ ID NO: 758) | |
| IHH | |
| (SEQ ID NO: 759) | |
| LGNIH | |
| (SEQ ID NO: 760) | |
| GNIH | |
| (SEQ ID NO: 761) | |
| NIH | |
| (SEQ ID NO: 762) | |
| LGNI | |
| (SEQ ID NO: 763) | |
| GNI | |
| (SEQ ID NO: 764) | |
| LGN | |
| (SEQ ID NO: 765) | |
| LDNITHVPGGG | |
| (SEQ ID NO: 766) | |
| DNITHVPGGG | |
| (SEQ ID NO: 767) | |
| NITHVPGGG | |
| (SEQ ID NO: 768) | |
| ITHVPGGG | |
| (SEQ ID NO: 769) | |
| THVPGGG | |
| (SEQ ID NO: 770) | |
| LDNITHVPGG | |
| (SEQ ID NO: 771) | |
| DNITHVPGG | |
| (SEQ ID NO: 772) | |
| NITHVPGG | |
| (SEQ ID NO: 773) | |
| ITHVPGG | |
| (SEQ ID NO: 774) | |
| THVPGG | |
| (SEQ ID NO: 775) | |
| LDNITHVPG | |
| (SEQ ID NO: 776) | |
| DNITHVPG | |
| (SEQ ID NO: 777) | |
| NITHVPG | |
| (SEQ ID NO: 778) | |
| ITHVPG | |
| (SEQ ID NO: 779) | |
| THVPG | |
| (SEQ ID NO: 780) | |
| LDNITHVP | |
| (SEQ ID NO: 781) | |
| DNITHVP | |
| (SEQ ID NO: 782) | |
| NITHVP | |
| (SEQ ID NO: 783) | |
| ITHVP | |
| (SEQ ID NO: 784) | |
| THVP | |
| (SEQ ID NO: 785) | |
| LDNITHV | |
| (SEQ ID NO: 786) | |
| DNITHV | |
| (SEQ ID NO: 787) | |
| NITHV | |
| (SEQ ID NO: 788) | |
| ITHV | |
| (SEQ ID NO: 789) | |
| THV | |
| (SEQ ID NO: 790) | |
| LDNITH | |
| (SEQ ID NO: 791) | |
| DNITH | |
| (SEQ ID NO: 792) | |
| NITH | |
| (SEQ ID NO: 793) | |
| ITH | |
| (SEQ ID NO: 794) | |
| LDNIT | |
| (SEQ ID NO: 795) | |
| DNIT | |
| (SEQ ID NO: 796) | |
| NIT | |
| (SEQ ID NO: 797) | |
| LDNI | |
| (SEQ ID NO: 798) | |
| LDN | |
| (SEQ ID NO: 799) | |
| KNVKSKIGST | |
| (SEQ ID NO: 800) | |
| NVKSKIGST | |
| (SEQ ID NO: 801) | |
| VKSKIGST | |
| (SEQ ID NO: 802) | |
| KSKIGST | |
| (SEQ ID NO: 803) | |
| SKIGST | |
| (SEQ ID NO: 804) | |
| KIGST | |
| (SEQ ID NO: 805) | |
| IGST | |
| (SEQ ID NO: 806) | |
| GST | |
| (SEQ ID NO: 807) | |
| NVKSKIGSTE | |
| (SEQ ID NO: 808) | |
| VKSKIGSTE | |
| (SEQ ID NO: 809) | |
| KSKIGSTE | |
| (SEQ ID NO: 810) | |
| SKIGSTE | |
| (SEQ ID NO: 811) | |
| KIGSTE | |
| (SEQ ID NO: 812) | |
| IGSTE | |
| (SEQ ID NO: 813) | |
| GSTE | |
| (SEQ ID NO: 814) | |
| STE | |
| (SEQ ID NO: 815) | |
| VKSKIGSTEN | |
| (SEQ ID NO: 816) | |
| KSKIGSTEN | |
| (SEQ ID NO: 817) | |
| SKIGSTEN | |
| (SEQ ID NO: 818) | |
| KIGSTEN | |
| (SEQ ID NO: 819) | |
| IGSTEN | |
| (SEQ ID NO: 820) | |
| GSTEN | |
| (SEQ ID NO: 821) | |
| STEN | |
| (SEQ ID NO: 822) | |
| KSKIGSTENL | |
| (SEQ ID NO: 823) | |
| SKIGSTENL | |
| (SEQ ID NO: 824) | |
| KIGSTENL | |
| (SEQ ID NO: 825) | |
| IGSTENL | |
| (SEQ ID NO: 826) | |
| GSTENL | |
| (SEQ ID NO: 827) | |
| STENL | |
| (SEQ ID NO: 828) | |
| SKIGSTENLK | |
| (SEQ ID NO: 829) | |
| KIGSTENLK | |
| (SEQ ID NO: 830) | |
| IGSTENLK | |
| (SEQ ID NO: 831) | |
| GSTENLK | |
| (SEQ ID NO: 832) | |
| STENLK | |
| (SEQ ID NO: 833) | |
| KIGSTENLKH | |
| (SEQ ID NO: 834) | |
| IGSTENLKH | |
| (SEQ ID NO: 835) | |
| GSTENLKH | |
| (SEQ ID NO: 836) | |
| STENLKH | |
| (SEQ ID NO: 837) | |
| IGSTENLKHQ | |
| (SEQ ID NO: 838) | |
| GSTENLKHQ | |
| (SEQ ID NO: 839) | |
| STENLKHQ | |
| (SEQ ID NO: 840) | |
| GSTENLKHQP | |
| (SEQ ID NO: 841) | |
| STENLKHQP | |
| (SEQ ID NO: 842) | |
| STENLKHQPG | |
| (SEQ ID NO: 843) | |
| SNVQSKCGSK | |
| (SEQ ID NO: 844) | |
| NVQSKCGSK | |
| (SEQ ID NO: 845) | |
| VQSKCGSK | |
| (SEQ ID NO: 846) | |
| QSKCGSK | |
| (SEQ ID NO: 847) | |
| SKCGSK | |
| (SEQ ID NO: 848) | |
| KCGSK | |
| (SEQ ID NO: 849) | |
| CGSK | |
| (SEQ ID NO: 850) | |
| GSK | |
| (SEQ ID NO: 851) | |
| NVQSKCGSKD | |
| (SEQ ID NO: 852) | |
| VQSKCGSKD | |
| (SEQ ID NO: 853) | |
| QSKCGSKD | |
| (SEQ ID NO: 854) | |
| SKCGSKD | |
| (SEQ ID NO: 855) | |
| KCGSKD | |
| (SEQ ID NO: 856) | |
| CGSKD | |
| (SEQ ID NO: 857) | |
| GSKD | |
| (SEQ ID NO: 858) | |
| SKD | |
| (SEQ ID NO: 859) | |
| VQSKCGSKDN | |
| (SEQ ID NO: 860) | |
| QSKCGSKDN | |
| (SEQ ID NO: 861) | |
| SKCGSKDN | |
| (SEQ ID NO: 862) | |
| KCGSKDN | |
| (SEQ ID NO: 863) | |
| CGSKDN | |
| (SEQ ID NO: 864) | |
| GSKDN | |
| (SEQ ID NO: 865) | |
| SKDN | |
| (SEQ ID NO: 866) | |
| QSKCGSKDNI | |
| (SEQ ID NO: 867) | |
| SKCGSKDNI | |
| (SEQ ID NO: 868) | |
| KCGSKDNI | |
| (SEQ ID NO: 869) | |
| CGSKDNI | |
| (SEQ ID NO: 870) | |
| GSKDNI | |
| (SEQ ID NO: 871) | |
| SKDNI | |
| (SEQ ID NO: 872) | |
| SKVTSKCGSL | |
| (SEQ ID NO: 873) | |
| KVTSKCGSL | |
| (SEQ ID NO: 874) | |
| VTSKCGSL | |
| (SEQ ID NO: 875) | |
| TSKCGSL | |
| (SEQ ID NO: 876) | |
| SKCGSL | |
| (SEQ ID NO: 877) | |
| KCGSL | |
| (SEQ ID NO: 878) | |
| CGSL | |
| (SEQ ID NO: 879) | |
| GSL | |
| (SEQ ID NO: 880) | |
| KVTSKCGSLG | |
| (SEQ ID NO: 881) | |
| VTSKCGSLG | |
| (SEQ ID NO: 882) | |
| TSKCGSLG | |
| (SEQ ID NO: 883) | |
| SKCGSLG | |
| (SEQ ID NO: 884) | |
| KCGSLG | |
| (SEQ ID NO: 885) | |
| CGSLG | |
| (SEQ ID NO: 886) | |
| GSLG | |
| (SEQ ID NO: 887) | |
| SLG | |
| (SEQ ID NO: 888) | |
| VTSKCGSLGN | |
| (SEQ ID NO: 889) | |
| TSKCGSLGN | |
| (SEQ ID NO: 890) | |
| SKCGSLGN | |
| (SEQ ID NO: 891) | |
| KCGSLGN | |
| (SEQ ID NO: 892) | |
| CGSLGN | |
| (SEQ ID NO: 893) | |
| GSLGN | |
| (SEQ ID NO: 894) | |
| SLGN | |
| (SEQ ID NO: 895) | |
| TSKCGSLGNI | |
| (SEQ ID NO: 896) | |
| SKCGSLGNI | |
| (SEQ ID NO: 897) | |
| KCGSLGNI | |
| (SEQ ID NO: 898) | |
| CGSLGNI | |
| (SEQ ID NO: 899) | |
| GSLGNI | |
| (SEQ ID NO: 900) | |
| SLGNI | |
| (SEQ ID NO: 901) | |
| SKCGSLGNIH | |
| (SEQ ID NO: 902) | |
| KCGSLGNIH | |
| (SEQ ID NO: 903) | |
| CGSLGNIH | |
| (SEQ ID NO: 904) | |
| GSLGNIH | |
| (SEQ ID NO: 905) | |
| SLGNIH | |
| (SEQ ID NO: 906) | |
| KCGSLGNIHH | |
| (SEQ ID NO: 907) | |
| CGSLGNIHH | |
| (SEQ ID NO: 908) | |
| GSLGNIHH | |
| (SEQ ID NO: 909) | |
| SLGNIHH | |
| (SEQ ID NO: 910) | |
| CGSLGNIHHK | |
| (SEQ ID NO: 911) | |
| GSLGNIHHK | |
| (SEQ ID NO: 912) | |
| SLGNIHHK | |
| (SEQ ID NO: 913) | |
| GSLGNIHHKP | |
| (SEQ ID NO: 914) | |
| SLGNIHHKP | |
| (SEQ ID NO: 915) | |
| SLGNIHHKPG | |
| (SEQ ID NO: 916) | |
| DRVQSKIGSL | |
| (SEQ ID NO: 917) | |
| RVQSKIGSL | |
| (SEQ ID NO: 918) | |
| VQSKIGSL | |
| (SEQ ID NO: 919) | |
| QSKIGSL | |
| (SEQ ID NO: 920) | |
| SKIGSL | |
| (SEQ ID NO: 921) | |
| KIGSL | |
| (SEQ ID NO: 922) | |
| IGSL | |
| (SEQ ID NO: 923) | |
| RVQSKIGSLD | |
| (SEQ ID NO: 924) | |
| VQSKIGSLD | |
| (SEQ ID NO: 925) | |
| QSKIGSLD | |
| (SEQ ID NO: 926) | |
| SKIGSLD | |
| (SEQ ID NO: 927) | |
| KIGSLD | |
| (SEQ ID NO: 928) | |
| IGSLD | |
| (SEQ ID NO: 929) | |
| GSLD | |
| (SEQ ID NO: 930) | |
| SLD | |
| (SEQ ID NO: 931) | |
| VQSKIGSLDN | |
| (SEQ ID NO: 932) | |
| QSKIGSLDN | |
| (SEQ ID NO: 933) | |
| SKIGSLDN | |
| (SEQ ID NO: 934) | |
| KIGSLDN | |
| (SEQ ID NO: 935) | |
| IGSLDN | |
| (SEQ ID NO: 936) | |
| GSLDN | |
| (SEQ ID NO: 937) | |
| SLDN | |
| (SEQ ID NO: 938) | |
| QSKIGSLDNI | |
| (SEQ ID NO: 939) | |
| SKIGSLDNI | |
| (SEQ ID NO: 940) | |
| KIGSLDNI | |
| (SEQ ID NO: 941) | |
| IGSLDNI | |
| (SEQ ID NO: 942) | |
| GSLDNI | |
| (SEQ ID NO: 943) | |
| SKIGSLDNIT | |
| (SEQ ID NO: 944) | |
| KIGSLDNIT | |
| (SEQ ID NO: 945) | |
| IGSLDNIT | |
| (SEQ ID NO: 946) | |
| GSLDNIT | |
| (SEQ ID NO: 947) | |
| SLDNIT | |
| (SEQ ID NO: 948) | |
| KIGSLDNITH | |
| (SEQ ID NO: 949) | |
| IGSLDNITH | |
| (SEQ ID NO: 950) | |
| GSLDNITH | |
| (SEQ ID NO: 951) | |
| SLDNITH | |
| (SEQ ID NO: 952) | |
| IGSLDNITHV | |
| (SEQ ID NO: 953) | |
| GSLDNITHV | |
| (SEQ ID NO: 954) | |
| SLDNITHV | |
| (SEQ ID NO: 955) | |
| GSLDNITHVP | |
| (SEQ ID NO: 956) | |
| SLDNITHVP | |
| (SEQ ID NO: 957) | |
| SLDNITHVPG | |
| (SEQ ID NO: 958) | |
| PDLKNVKS | |
| (SEQ ID NO: 959) | |
| DLKNVKSK | |
| (SEQ ID NO: 960) | |
| LKNVKSKI | |
| (SEQ ID NO: 961) | |
| KNVKSKIG | |
| (SEQ ID NO: 962) | |
| NVKSKIGS | |
| (SEQ ID NO: 963) | |
| DLSNVQSK | |
| (SEQ ID NO: 964) | |
| LSNVQSKC | |
| (SEQ ID NO: 965) | |
| SNVQSKCG | |
| (SEQ ID NO: 966) | |
| NVQSKCGS | |
| (SEQ ID NO: 967) | |
| VDLSKVTS | |
| (SEQ ID NO: 968) | |
| DLSKVTSK | |
| (SEQ ID NO: 969) | |
| LSKVTSKC | |
| (SEQ ID NO: 970) | |
| SKVTSKCG | |
| (SEQ ID NO: 971) | |
| KVTSKCGS | |
| (SEQ ID NO: 972) | |
| LDFKDRVQ | |
| (SEQ ID NO: 973) | |
| DFKDRVQS | |
| (SEQ ID NO: 974) | |
| FKDRVQSK | |
| (SEQ ID NO: 975) | |
| KDRVQSKI | |
| (SEQ ID NO: 976) | |
| DRVQSKIG | |
| (SEQ ID NO: 977) | |
| RVQSKIGS | |
| (SEQ ID NO: 978) | |
| SKIGSTENLKH | |
| (SEQ ID NO: 979) | |
| SKIGSTENIKH | |
| (SEQ ID NO: 980) | |
| SKIGSKDNLKH | |
| (SEQ ID NO: 981) | |
| SKIGSKENIKH | |
| (SEQ ID NO: 982) | |
| SKIGSLENLKH | |
| (SEQ ID NO: 983) | |
| SKIGSLENIKH | |
| (SEQ ID NO: 984) | |
| SKIGSTDNLKH | |
| (SEQ ID NO: 985) | |
| SKIGSTDNIKH | |
| (SEQ ID NO: 986) | |
| SKIGSKDNIKH | |
| (SEQ ID NO: 987) | |
| SKIGSLDNLKH | |
| (SEQ ID NO: 988) | |
| SKIGSLDNIKH | |
| (SEQ ID NO: 989) | |
| SKIGSTGNLKH | |
| (SEQ ID NO: 990) | |
| SKIGSTGNIKH | |
| (SEQ ID NO: 991) | |
| SKIGSKGNLKH | |
| (SEQ ID NO: 992) | |
| SKIGSKGNIKH | |
| (SEQ ID NO: 993) | |
| SKIGSLGNLKH | |
| (SEQ ID NO: 994) | |
| SKIGSLGNIKH | |
| (SEQ ID NO: 995) | |
| Lys Xaa₁ Xaa₂ Ser Xaa₃ Xaa₄ Asn Xaa₅ Xaa₆ His, | |
| wherein | |
| Xaa₁ is I or C; | |
| Xaa₂ is G; | |
| Xaa₃ is T, K or L; | |
| Xaa₄ is E, D or G; | |
| Xaa₅ is L or I; | |
| Xaa₆ is K, H or T. | |
| (SEQ ID NO: 996) | |
| (Q/E)IVYK(S/P) | |
| alpha-synuclein isoform NACP140 [Homo sapiens] | |
| NCBI Reference Sequence: NP_000336.1 | |
| (SEQ ID NO: 58) | |
| 1 | MDVFMKGLSK AKEGVVAAAE KTKQGVAEAA GKTKEGVLYV GSKTKEGVVH GVATVAEKTK |
| 61 | EQVTNVGGAV VTGVTAVAQK TVEGAGSIAA ATGFVKKDQL GKNEEGAPQE GILEDMPVDP |
| 121 | DNEAYEMPSE EGYQDYEPEA |

(SEQ ID NO: 59)
VDPDNEAYEM
(SEQ ID NO: 60)
VDPDNEAYE
(SEQ ID NO: 61)
VDPDNEAY
(SEQ ID NO: 62)
VDPDNEA
(SEQ ID NO: 63)
VDPDNE
(SEQ ID NO: 64)
VDPDN
(SEQ ID NO: 65)
VDPD
(SEQ ID NO: 66)
VDP
(SEQ ID NO: 67)
DPDNEAYEM
(SEQ ID NO: 68)
DPDNEAYE
(SEQ ID NO: 69)
DPDNEAY
(SEQ ID NO: 70)
DPDNEA
(SEQ ID NO: 71)
DPDNE
(SEQ ID NO: 72)
DPDN
(SEQ ID NO: 73)
DPD
(SEQ ID NO: 74)
PDNEAYEM
(SEQ ID NO: 75)
PDNEAYE
(SEQ ID NO: 76)
PDNEAY
(SEQ ID NO: 77)
PDNEA
(SEQ ID NO: 78)
PDNE
(SEQ ID NO: 79)
PDN
(SEQ ID NO: 80)
DNEAYEM
(SEQ ID NO: 81)
DNEAYE
(SEQ ID NO: 82)
DNEAY
(SEQ ID NO: 83)
DNEA
(SEQ ID NO: 84)
DNE
(SEQ ID NO: 85)
NEAYEM
(SEQ ID NO: 86)
NEAYE
(SEQ ID NO: 87)
NEAY
(SEQ ID NO: 88)
NEA
(SEQ ID NO: 89)
EAYEM
(SEQ ID NO: 90)
EAYE
(SEQ ID NO: 91)
EAY
(SEQ ID NO: 92)
AYEM
(SEQ ID NO: 93)
AYE
(SEQ ID NO: 94)
YEM
(SEQ ID NO: 95)
ATGFVKKDQL
(SEQ ID NO: 96)
ATGFVKKDQ
(SEQ ID NO: 97)
ATGFVKKD
(SEQ ID NO: 98)
ATGFVKK
(SEQ ID NO: 99)
ATGFVK
(SEQ ID NO: 100)
ATGFV
(SEQ ID NO: 101)
ATGF
(SEQ ID NO: 102)
ATG
(SEQ ID NO: 103)
TGFVKKDQL
(SEQ ID NO: 104)
TGFVKKDQ
(SEQ ID NO: 105)
TGFVKKD
(SEQ ID NO: 106)
TGFVKK
(SEQ ID NO: 107)
TGFVK
(SEQ ID NO: 108)
TGFV
(SEQ ID NO: 109)
TGF
(SEQ ID NO: 110)
GFVKKDQL
(SEQ ID NO: 111)
GFVKKDQ
(SEQ ID NO: 112)
GFVKKD
(SEQ ID NO: 113)
GFVKK
(SEQ ID NO: 114)
GFVK
(SEQ ID NO: 115)
GFV
(SEQ ID NO: 116)
FVKKDQL
(SEQ ID NO: 117)
FVKKDQ
(SEQ ID NO: 118)
FVKKD
(SEQ ID NO: 119)
FVKK
(SEQ ID NO: 120)
FVK
(SEQ ID NO: 121)
VKKDQL
(SEQ ID NO: 122)
VKKDQ
(SEQ ID NO: 123)
VKKD
(SEQ ID NO: 124)
VKK
(SEQ ID NO: 125)
KKDQL
(SEQ ID NO: 126)
KKDQ
(SEQ ID NO: 127)
KKD
(SEQ ID NO: 128)
KDQL
(SEQ ID NO: 129)
KDQ
(SEQ ID NO: 130)
DAEFRHDRRPDNEAYERRQIVYKPVKKC
(SEQ ID NO: 131)
DAEFRHDRRQIVYKPVRRPDNEAYEKKC
(SEQ ID NO: 132)
DAEFRHDRRPDNEAYERRNIKHVPGKKC
(SEQ ID NO: 133)
DAEFRHDRRNIKHVPGRRPDNEAYEKKC
(SEQ ID NO: 134)
DAEFRHDRRQIVYKPVRRPDNEAYERRNIKHVPGGC
(SEQ ID NO: 135)
DAEFRHDRRDPDNEAYRRNIKHVPGRRQIVYKPVGGC
(SEQ ID NO: 136)
EFRHDSGRRQIVYKPVRRPDNEAYERRNIKHVPGGC
(SEQ ID NO: 137)
EFRHDSGRRDPDNEAYRRNIKHVPGRRQIVYKPVGGC
(SEQ ID NO: 1058)
DAEFRHDRRDPDNEAYERRENLKHQPGGGC
(SEQ ID NO: 1059)
DAEFRHDRRENLKHQPGRRDPDNEAYEGGC
(SEQ ID NO: 1060)
DAEFRHDRRPDNEAYERRENLKHQPGGGC
(SEQ ID NO: 1061)
DAEFRHDRRENLKHQPGRRPDNEAYEGGC
(SEQ ID NO: 1062)
DAEFRHDRRSKIGSKDNIKHRRDPDNEAYEGGC
(SEQ ID NO: 1063)
DAEFRHDRRDPDNEAYERRSKIGSKDNIKHGGC
(RVRR; SEQ ID NO: 138)
Arg-Val-Arg-Arg
(GAGA; SEQ ID NO: 139)
Gly-Ala-Gly-Ala
(AGAG; SEQ ID NO: 140)
Ala-Gly-Ala-Gly
(KGKG; SEQ ID NO: 141)
Lys-Gly-Lys-Gly
(SEQ ID NO: 142)
AEFRHDSGC
(SEQ ID NO: 143)
DAEFRHDC
(SEQ ID NO: 144)
CPDNEAYE
(SEQ ID NO: 145)
DPDNEAYC
(SEQ ID NO: 1064)
GGGS
(SEQ ID NO: 1065)
GGGGS

1. A polypeptide comprising:
(a) a first peptide comprising 3-10 amino acids from residues 1-10 or 12-25 of SEQ ID NO:01;
(b) a second peptide comprising 3-13 amino acids from residues 244-400 of SEQ ID NO:02; and
(c) a third peptide comprising 3-10 amino acids from residues 81-140 of SEQ ID NO:58;
wherein the first peptide, second peptide and third peptide are arranged in any order in the polypeptide.

2. The polypeptide of claim 1, further comprising a fourth peptide selected from one of:
(a) a peptide comprising 3-10 amino acids from residues 1-10 or 12-25 of SEQ ID NO:01;
(b) a peptide comprising 3-13 amino acids from residues 244-400 of SEQ ID NO:02; and
(c) a peptide comprising 3-10 amino acids from residues 81-140 of SEQ ID NO:58;
wherein the first peptide, second peptide, third peptide, and fourth peptide are arranged in any order in the polypeptide.

3. The polypeptide of claim 1, wherein the second peptide is from the microtubule binding region (MTBR) of tau (residues 244-372 of SEQ ID NO:02).

4. The polypeptide of claim 1, wherein:
(a) the first peptide comprises an amino acid sequence selected from the group consisting of
(SEQ ID NO: 03)
DAEFRHDSGY,
(SEQ ID NO: 04)
DAEFRHDSG,
(SEQ ID NO: 05)
DAEFRHDS,
(SEQ ID NO: 06)
DAEFRHD,
(SEQ ID NO: 07)
DAEFRH,
(SEQ ID NO: 08)
DAEFR,
(SEQ ID NO: 09)
DAEF,
(SEQ ID NO: 10)
DAE,
(SEQ ID NO: 11)
AEFRHDSGY,
(SEQ ID NO: 12)
AEFRHDSG,
(SEQ ID NO: 13)
AEFRHDS,
(SEQ ID NO: 14)
AEFRHD,
(SEQ ID NO: 15)
AEFRH,
(SEQ ID NO: 16)
AEFR,
(SEQ ID NO: 17)
AEF,
(SEQ ID NO: 18)
EFRHDSGY,
(SEQ ID NO: 19)
EFRHDSG,
(SEQ ID NO: 20)
EFRHDS,
(SEQ ID NO: 21)
EFRHD,
(SEQ ID NO: 22)
EFRH,
(SEQ ID NO: 23)
EFR,
(SEQ ID NO: 24)
FRHDSGY,
(SEQ ID NO: 25)
FRHDSG,
(SEQ ID NO: 26)
FRHDS,
(SEQ ID NO: 27)
FRHD,
(SEQ ID NO: 28)
FRH,
(SEQ ID NO: 29)
RHDSGY,
(SEQ ID NO: 30)
RHDSG,
(SEQ ID NO: 31)
RHDS,
(SEQ ID NO: 32)
RHD,
(SEQ ID NO: 33)
HDSGY,
(SEQ ID NO: 34)
HDSG,
(SEQ ID NO: 35)
HDS,
(SEQ ID NO: 36)
DSGY,
(SEQ ID NO: 37)
DSG,
(SEQ ID NO: 38)
SGY,
(SEQ ID NO: 1002)
VHHQKLVFFA,
(SEQ ID NO: 1003)
VHHQKLVFF,
(SEQ ID NO: 1004)
VHHQKLVF,
(SEQ ID NO: 1005)
VHHQKLV,
(SEQ ID NO: 1006)
VHHQKL,
(SEQ ID NO: 1007)
HHQKLVFFAE,
(SEQ ID NO: 1008)
HHQKLVFFA,
(SEQ ID NO: 1009)
HHQKLVFF,
(SEQ ID NO: 1010)
HHQKLVF,
(SEQ ID NO: 1011)
HHQKLV,
(SEQ ID NO: 1012)
HHQKL,
(SEQ ID NO: 1013)
HQKLVFFAED,
(SEQ ID NO: 1014)
HQKLVFFAE,
(SEQ ID NO: 1015)
HQKLVFFA,
(SEQ ID NO: 1016)
HQKLVFF,
(SEQ ID NO: 1017)
HQKLVF,
(SEQ ID NO: 1018)
HQKLV,
(SEQ ID NO: 1019)
HQKL,
(SEQ ID NO: 1020)
QKLVFFAEDV,
(SEQ ID NO: 1021)
QKLVFFAED,
(SEQ ID NO: 1022)
QKLVFFAE,
(SEQ ID NO: 1023)
QKLVFFA,
(SEQ ID NO: 1024)
QKLVFF,
(SEQ ID NO: 1025)
QKLVF,
(SEQ ID NO: 1026)
QKLV,
(SEQ ID NO: 1027)
QKL,
(SEQ ID NO: 1028)
KLVFFAEDVG,
(SEQ ID NO: 1029)
KLVFFAEDV,
(SEQ ID NO: 1030)
KLVFFAED,
(SEQ ID NO: 1031)
KLVFFAE,
(SEQ ID NO: 1032)
KLVFFA,
(SEQ ID NO: 1033)
KLVFF,
(SEQ ID NO: 1034)
KLVF,
(SEQ ID NO: 1035)
KLV,
(SEQ ID NO: 1036)
LVFFAEDVG,
(SEQ ID NO: 1037)
LVFFAEDV,
(SEQ ID NO: 1038)
LVFFAED,
(SEQ ID NO: 1039)
LVFFAE,
(SEQ ID NO: 1040)
LVFFA,
(SEQ ID NO: 1041)
LVFF,
(SEQ ID NO: 1042)
LVF,
(SEQ ID NO: 1043)
VFFAEDVG,
(SEQ ID NO: 1044)
VFFAEDV,
(SEQ ID NO: 1045)
VFFAED,
(SEQ ID NO: 1046)
VFFAE,
(SEQ ID NO: 1047)
VFFA,
(SEQ ID NO: 1048)
VFF,
(SEQ ID NO: 1049)
FFAEDVG,
(SEQ ID NO: 1050)
FFAEDV,
(SEQ ID NO: 1051)
FFAED,
(SEQ ID NO: 1052)
FFAE,
(SEQ ID NO: 1053)
FFA,
(SEQ ID NO: 1054)
FAEDVG,
(SEQ ID NO: 1055)
FAEDV,
(SEQ ID NO: 1056)
FAED,
and
(SEQ ID NO: 1057)
FAE;
(b) the second peptide comprises an amino acid sequence selected from the group consisting of
(SEQ ID NO: 39)
QIVYKPV,
(SEQ ID NO: 40)
QIVYKP,
(SEQ ID NO: 41)
QIVYKSV,
(SEQ ID NO: 42)
EIVYKSV,
(SEQ ID NO: 997)
QIVYKS,
(SEQ ID NO: 43)
EIVYKSP,
(SEQ ID NO: 998)
EIVYKS,
(SEQ ID NO: 44)
EIVYKPV,
(SEQ ID NO: 999)
EIVYKP,
(SEQ ID NO: 45)
IVYKSPV,
(SEQ ID NO: 46)
IVYK,
(SEQ ID NO: 1000)
CNIKHVPG,
(SEQ ID NO: 47)
CNIKHVP,
(SEQ ID NO: 48)
NIKHVP,
(SEQ ID NO: 49)
HVPGGG,
(SEQ ID NO: 50)
HVPGG,
(SEQ ID NO: 51)
HKPGGG,
(SEQ ID NO: 52)
HKPGG,
SEQ ID NO: 53)
KHVPGGG,
(SEQ ID NO: 54)
KHVPGG,
(SEQ ID NO: 55)
HQPGGG,
(SEQ ID NO: 56)
HQPGG,
(SEQ ID NO: 57)
NIKHVPG,
and
SEQ ID NOs: 146-996;
(c) the third peptide comprises an amino acid sequence selected from the group consisting of
(SEQ ID NO: 59)
VDPDNEAYEM,
(SEQ ID NO: 60)
VDPDNEAYE,
(SEQ ID NO: 61)
VDPDNEAY,
(SEQ ID NO: 62)
VDPDNEA,
(SEQ ID NO: 63)
VDPDNE,
(SEQ ID NO: 64)
VDPDN,
(SEQ ID NO: 65)
VDPD,
(SEQ ID NO: 66)
VDP,
(SEQ ID NO: 67)
DPDNEAYEM,
(SEQ ID NO: 68)
DPDNEAYE,
(SEQ ID NO: 69)
DPDNEAY,
(SEQ ID NO: 70)
DPDNEA,
(SEQ ID NO: 71)
DPDNE,
(SEQ ID NO: 72)
DPDN,
(SEQ ID NO: 73)
DPD,
(SEQ ID NO: 74)
PDNEAYEM,
(SEQ ID NO: 75)
PDNEAYE,
(SEQ ID NO: 76)
PDNEAY,
(SEQ ID NO: 77)
PDNEA,
(SEQ ID NO: 78)
PDNE,
(SEQ ID NO: 79)
PDN,
(SEQ ID NO: 80)
DNEAYEM,
(SEQ ID NO: 81)
DNEAYE,
(SEQ ID NO: 82)
DNEAY,
(SEQ ID NO: 83)
DNEA,
(SEQ ID NO: 84)
DNE,
(SEQ ID NO: 85)
NEAYEM,
(SEQ ID NO: 86)
NEAYE,
(SEQ ID NO: 87)
NEAY,
(SEQ ID NO: 88)
NEA,
(SEQ ID NO: 89)
EAYEM,
(SEQ ID NO: 90)
EAYE,
(SEQ ID NO: 91)
EAY,
(SEQ ID NO: 92)
AYEM,
(SEQ ID NO: 93)
AYE
(SEQ ID NO: 94)
YEM,
(SEQ ID NO: 95)
ATGFVKKDQL,
(SEQ ID NO: 96)
ATGFVKKDQ,
(SEQ ID NO: 97)
ATGFVKKD,
(SEQ ID NO: 98)
ATGFVKK,
(SEQ ID NO: 99)
ATGFVK,
(SEQ ID NO: 100)
ATGFV,
(SEQ ID NO: 101)
ATGF,
(SEQ ID NO: 102)
ATG,
(SEQ ID NO: 103)
TGFVKKDQL,
(SEQ ID NO: 104)
TGFVKKDQ,
(SEQ ID NO: 105)
TGFVKKD,
(SEQ ID NO: 106)
TGFVKK,
(SEQ ID NO: 107)
TGFVK,
(SEQ ID NO: 108)
TGFV,
(SEQ ID NO: 109)
TGF,
(SEQ ID NO: 110)
GFVKKDQL,
(SEQ ID NO: 111)
GFVKKDQ,
(SEQ ID NO: 112)
GFVKKD,
(SEQ ID NO: 113)
GFVKK,
(SEQ ID NO: 114)
GFVK,
(SEQ ID NO: 115)
GFV,
(SEQ ID NO: 116)
FVKKDQL,
(SEQ ID NO: 117)
FVKKDQ,
(SEQ ID NO: 118)
FVKKD,
(SEQ ID NO: 119)
FVKK,
(SEQ ID NO: 120)
FVK,
(SEQ ID NO: 121)
VKKDQL,
(SEQ ID NO: 122)
VKKDQ,
(SEQ ID NO: 123)
VKKD,
(SEQ ID NO: 124)
VKK,
(SEQ ID NO: 125)
KKDQL,
(SEQ ID NO: 126)
KKDQ,
(SEQ ID NO: 127)
KKD,
(SEQ ID NO: 128)
KDQL,
and
(SEQ ID NO: 129)
KDQ;
and
(d) optionally comprising a fourth polypeptide selected from an amino acid sequence of SEQ ID NO: 3-38, 1002-1057, 39-57, 142-1000, and 59-129.

5. The polypeptide of claim 4, wherein two or more of the first peptide, the second peptide, and the third peptide, and the fourth peptide, if present, are each linked by a cleavable linker.

6. The polypeptide of claim 5, wherein each of the cleavable linkers comprise an amino acid sequence.

7. The polypeptide of claim 6, wherein the amino acid sequence of each cleavable linker is independently selected from the group consisting of: arginine-arginine (Arg-Arg), arginine-valine-arginine-arginine (Arg-Val-Arg-Arg; SEQ ID NO:138), valine-citrulline (Val-Cit), valine-arginine (Val-Arg), valine-lysine (Val-Lys), valine-alanine (Val-Ala), phenylalanine-lysine (Phe-Lys), glycine-alanine-glycine-alanine (Gly-Ala-Gly-Ala; SEQ ID NO:139), alanine-glycine-alanine-glycine (Ala-Gly-Ala-Gly; SEQ ID NO:140), and lysine-glycine-lysine-glycine (Lys-Gly-Lys-Gly; SEQ ID NO:141).

8. The polypeptide of claim 1, further comprising a linker to a carrier at either a C-terminal portion of the polypeptide, or a N-terminal portion of the polypeptide.

9. The polypeptide of claim 8, wherein the linker to a carrier comprises an amino acid sequence selected from the group consisting of GG, GGG, AA, AAA, KK, KKK, SS, SSS, GAGA (SEQ ID NO:139), AGAG (SEQ ID NO:140), and KGKG (SEQ ID NO:141).

10. The polypeptide of claim 8, wherein the polypeptide or linker to the carrier further comprises a C-terminal cysteine (C).

11. The polypeptide of claim 1, wherein the polypeptide further comprises a blocked amine at the N-terminus.

12. The polypeptide of claim 4, wherein any of the first peptide, second peptide, third peptide, or fourth peptide, if present, comprises 5-10 amino acids.

13. The polypeptide of claim 1, wherein the first peptide is DAEFRHD (SEQ ID NO:06).

14. The polypeptide of claim 1, wherein the first peptide is EFRHDSG (SEQ ID NO:19).

15. The polypeptide of claim 1, wherein the second peptide comprises the amino acid sequence QIVYKPV (SEQ ID NO:39).

16. The polypeptide of claim 1, wherein the second peptide comprises the amino acid sequence NIKHVPG (SEQ ID NO:57).

17. The polypeptide of claim 1, wherein the third peptide comprises the amino acid sequence PDNEAYE (SEQ ID NO:75).

18. The polypeptide of claim 1, wherein the third peptide comprises the amino acid sequence DPDNEAY (SEQ ID NO:69).

19. The polypeptide of claim 1, wherein the fourth peptide comprises the amino acid sequence NIKHVP (SEQ ID NO:48).

20. The polypeptide of claim 1, wherein the fourth peptide comprises the amino acid sequence QIVYKPV (SEQ ID NO:39).

21. The polypeptide of claim 1, comprising an amino acid sequence of:
(SEQ ID NO: 130)
DAEFRHDRRPDNEAYERRQIVYKPVKKC;
(SEQ ID NO: 131)
DAEFRHDRRQIVYKPVRRPDNEAYEKKC;
(SEQ ID NO: 132)
DAEFRHDRRPDNEAYERRNIKHVPGKKC.
(SEQ ID NO: 133)
DAEFRHDRRNIKHVPGRRPDNEAYEKKC;,
(SEQ ID NO: 1058)
DAEFRHDRRDPDNEAYERRENLKHQPGGGC;
(SEQ ID NO: 1059)
DAEFRHDRRENLKHQPGRRDPDNEAYEGGC;
(SEQ ID NO: 1060)
DAEFRHDRRPDNEAYERRENLKHQPGGGC;
(SEQ ID NO: 1061)
DAEFRHDRRENLKHQPGRRPDNEAYEGGC;
(SEQ ID NO: 1062)
DAEFRHDRRSKIGSKDNIKHRRDPDNEAYEGGC;
or
(SEQ ID NO: 1063)
DAEFRHDRRDPDNEAYERRSKIGSKDNIKHGGC.

22. The polypeptide of claim 2, comprising an amino acid sequence of:
(SEQ ID NO: 134)
DAEFRHDRRQIVYKPVRRPDNEAYERRNIKHVPGGC;
(SEQ ID NO: 135)
DAEFRHDRRDPDNEAYRRNIKHVPGRRQIVYKPVGGC;
(SEQ ID NO: 136)
EFRHDSGRRQIVYKPVRRPDNEAYERRNIKHVPGGC;
or
(SEQ ID NO: 137)
EFRHDSGRRDPDNEAYRRNIKHVPGRRQIVYKPVGGC.

23. The polypeptide of claim 22, wherein the polypeptide is EFRHDSGRRQIVYKPVRRPDNEAYERRNIKHVPGGC (SEQ ID NO:136), and further comprises a blocked amine at the N-terminus.

24. The polypeptide of claim 22, wherein the polypeptide is EFRHDSGRRDPDNEAYRRNIKHVPGRRQIVYKPVGGC (SEQ ID NO:137), and further comprising a blocked amine at the N-terminus.

25. An immunotherapy composition, comprising the polypeptide of claim 1, wherein the polypeptide is linked to a carrier.

26. The immunotherapy composition of claim 25, wherein the carrier comprises serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

27. The immunotherapy composition of claim 26, wherein the carrier is CRM197.

28. The immunotherapy composition of claim 26, wherein the carrier is diphtheria toxoid.

29. A pharmaceutical formulation comprising (a) the polypeptide of claim 1 and (b) an adjuvant.

30. The pharmaceutical formulation of claim 29, wherein the adjuvant is selected from the group consisting of aluminum hydroxide, aluminum phosphate, aluminum sulfate, 3 De-O-acylated monophosphoryl lipid A (MPL), QS-21, TQL1055, QS-18, QS-17, QS-7, Complete Freund's Adjuvant (CFA), Incomplete Freund's Adjuvant (IFA), oil in water emulsions (such as squalene or peanut oil), CpG, polyglutamic acid, polylysine, AddaVax™, MF59®, and combinations thereof.

31. The pharmaceutical formulation of claim 30, wherein the adjuvant is QS-21 or TQL1055.

32. The pharmaceutical formulation of claim 30, wherein the adjuvant is MPL.

33. The pharmaceutical formulation of claim 30, wherein the adjuvant is a combination of MPL and QS-21 or a combination of MPL and TQL1055.

34. The pharmaceutical formulation of claim 29, wherein the adjuvant comprises a liposomal formulation.

35. The pharmaceutical formulation of claim 29, wherein the composition comprises at least one pharmaceutically acceptable diluent.

36. The pharmaceutical formulation of claim 29, comprising a multiple antigen presenting system (MAP).

37. The pharmaceutical formulation of claim 36, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

38. An immunotherapy composition, comprising:
(a) a first peptide sequence comprising 3-10 amino acid residues from the first ten or 12 to 25 N-terminal residues of SEQ ID NO:01;
(b) a second peptide sequence comprising 3-13 amino acids from residues 244-400 of SEQ ID NO:02; and
(c) a third peptide sequence comprising 3-10 amino acids from residues 81-140 of SEQ ID NO:58.

39. The polypeptide of claim 38, further comprising a fourth peptide sequence selected from one of:
(a) a peptide comprising 3-10 amino acids from residues 1-10 or 12-25 of SEQ ID NO:01;
(b) a peptide comprising 3-13 amino acids from residues 244-400 of SEQ ID NO:02; and
(c) a peptide comprising 3-10 amino acids from residues 81-140 of SEQ ID NO:58.

40. The immunotherapy composition of claim 38, wherein:
(a) the first peptide sequence comprises an amino acid sequence selected from the group consisting of
(SEQ ID NO: 03)
DAEFRHDSGY,
(SEQ ID NO: 04)
DAEFRHDSG,
(SEQ ID NO: 05)
DAEFRHDS,
(SEQ ID NO: 06)
DAEFRHD,
(SEQ ID NO: 07)
DAEFRH,
(SEQ ID NO: 08)
DAEFR,
(SEQ ID NO: 09)
DAEF,
(SEQ ID NO: 10)
DAE,
(SEQ ID NO: 11)
AEFRHDSGY,
(SEQ ID NO: 12)
AEFRHDSG,
(SEQ ID NO: 13)
AEFRHDS,
(SEQ ID NO: 14)
AEFRHD,
(SEQ ID NO: 15)
AEFRH,
(SEQ ID NO: 16)
AEFR,
(SEQ ID NO: 17)
AEF,
(SEQ ID NO: 18)
EFRHDSGY,
(SEQ ID NO: 19)
EFRHDSG,
(SEQ ID NO: 20)
EFRHDS,
(SEQ ID NO: 21)
EFRHD,
(SEQ ID NO: 22)
EFRH,
(SEQ ID NO: 23)
EFR,
(SEQ ID NO: 24)
FRHDSGY,
(SEQ ID NO: 25)
FRHDSG,
(SEQ ID NO: 26)
FRHDS,
(SEQ ID NO: 27)
FRHD,
(SEQ ID NO: 28)
FRH,
(SEQ ID NO: 29)
RHDSGY,
(SEQ ID NO: 30)
RHDSG,
(SEQ ID NO: 31)
RHDS,
(SEQ ID NO: 32)
RHD,
(SEQ ID NO: 33)
HDSGY,
(SEQ ID NO: 34)
HDSG,
(SEQ ID NO: 35)
HDS,
(SEQ ID NO: 36)
DSGY,
(SEQ ID NO: 37)
DSG,
(SEQ ID NO: 38)
SGY,
(SEQ ID NO: 1002)
VHHQKLVFFA,
(SEQ ID NO: 1003)
VHHQKLVFF,
(SEQ ID NO: 1004)
VHHQKLVF,
(SEQ ID NO: 1005)
VHHQKLV,
(SEQ ID NO: 1006)
VHHQKL,
(SEQ ID NO: 1007)
HHQKLVFFAE,
(SEQ ID NO: 1008)
HHQKLVFFA,
(SEQ ID NO: 1009)
HHQKLVFF,
(SEQ ID NO: 1010)
HHQKLVF,
(SEQ ID NO: 1011)
HHQKLV,
(SEQ ID NO: 1012)
HHQKL,
(SEQ ID NO: 1013)
HQKLVFFAED,
(SEQ ID NO: 1014)
HQKLVFFAE,
(SEQ ID NO: 1015)
HQKLVFFA,
(SEQ ID NO: 1016)
HQKLVFF,
(SEQ ID NO: 1017)
HQKLVF,
(SEQ ID NO: 1018)
HQKLV,
(SEQ ID NO: 1019)
HQKL,
(SEQ ID NO: 1020)
QKLVFFAEDV,
(SEQ ID NO: 1021)
QKLVFFAED,
(SEQ ID NO: 1022)
QKLVFFAE,
(SEQ ID NO: 1023)
QKLVFFA,
(SEQ ID NO: 1024)
QKLVFF,
(SEQ ID NO: 1025)
QKLVF,
(SEQ ID NO: 1026)
QKLV,
(SEQ ID NO: 1027)
QKL,
(SEQ ID NO: 1028)
KLVFFAEDVG,
(SEQ ID NO: 1029)
KLVFFAEDV,
(SEQ ID NO: 1030)
KLVFFAED,
(SEQ ID NO: 1031)
KLVFFAE,
(SEQ ID NO: 1032)
KLVFFA,
(SEQ ID NO: 1033)
KLVFF,
(SEQ ID NO: 1034)
KLVF,
(SEQ ID NO: 1035)
KLV,
(SEQ ID NO: 1036)
LVFFAEDVG,
(SEQ ID NO: 1037)
LVFFAEDV,
(SEQ ID NO: 1038)
LVFFAED,
(SEQ ID NO: 1039)
LVFFAE,
(SEQ ID NO: 1040)
LVFFA,
(SEQ ID NO: 1041)
LVFF,
(SEQ ID NO: 1042)
LVF,
(SEQ ID NO: 1043)
VFFAEDVG,
(SEQ ID NO: 1044)
VFFAEDV,
(SEQ ID NO: 1045)
VFFAED,
(SEQ ID NO: 1046)
VFFAE,
(SEQ ID NO: 1047)
VFFA,
(SEQ ID NO: 1048)
VFF,
(SEQ ID NO: 1049)
FFAEDVG,
(SEQ ID NO: 1050)
FFAEDV,
(SEQ ID NO: 1051)
FFAED,
(SEQ ID NO: 1052)
FFAE,
(SEQ ID NO: 1053)
FFA,
(SEQ ID NO: 1054)
FAEDVG,
(SEQ ID NO: 1055)
FAEDV,
(SEQ ID NO: 1056)
FAED,
and
(SEQ ID NO: 1057)
FAE;
(b) the second peptide sequence comprises an amino acid sequence selected from the group consisting of
(SEQ ID NO: 39)
QIVYKPV,
(SEQ ID NO: 40)
QIVYKP,
(SEQ ID NO: 41)
QIVYKSV,
(SEQ ID NO: 42)
EIVYKSV,
(SEQ ID NO: 997)
QIVYKS,
(SEQ ID NO: 43)
EIVYKSP,
(SEQ ID NO: 998)
EIVYKS,
(SEQ ID NO: 44)
EIVYKPV,
(SEQ ID NO: 999)
EIVYKP,
(SEQ ID NO: 45)
IVYKSPV,
(SEQ ID NO: 46)
IVYK,
(SEQ ID NO: 1000)
CNIKHVPG,
(SEQ ID NO: 47)
CNIKHVP,
(SEQ ID NO: 48)
NIKHVP,
(SEQ ID NO: 49)
HVPGGG,
(SEQ ID NO: 50)
HVPGG,
(SEQ ID NO: 51)
HKPGGG,
(SEQ ID NO: 52)
HKPGG,
SEQ ID NO: 53)
KHVPGGG,
(SEQ ID NO: 54)
KHVPGG,
(SEQ ID NO: 55)
HQPGGG,
(SEQ ID NO: 56)
HQPGG,
(SEQ ID NO: 57)
NIKHVPG,
and
SEQ ID NOs: 146-996;
and
(c) the third peptide sequence comprises an amino acid sequence selected from the group consisting of
(SEQ ID NO: 59)
VDPDNEAYEM,
(SEQ ID NO: 60)
VDPDNEAYE,
(SEQ ID NO: 61)
VDPDNEAY,
(SEQ ID NO: 62)
VDPDNEA,
(SEQ ID NO: 63)
VDPDNE,
(SEQ ID NO: 64)
VDPDN,
(SEQ ID NO: 65)
VDPD,
(SEQ ID NO: 66)
VDP,
(SEQ ID NO: 67)
DPDNEAYEM,
(SEQ ID NO: 68)
DPDNEAYE,
(SEQ ID NO: 69)
DPDNEAY,
(SEQ ID NO: 70)
DPDNEA,
(SEQ ID NO: 71)
DPDNE,
(SEQ ID NO: 72)
DPDN,
(SEQ ID NO: 73)
DPD,
(SEQ ID NO: 74)
PDNEAYEM,
(SEQ ID NO: 75)
PDNEAYE,
(SEQ ID NO: 76)
PDNEAY,
(SEQ ID NO: 77)
PDNEA,
(SEQ ID NO: 78)
PDNE,
(SEQ ID NO: 79)
PDN,
(SEQ ID NO: 80)
DNEAYEM,
(SEQ ID NO: 81)
DNEAYE,
(SEQ ID NO: 82)
DNEAY,
(SEQ ID NO: 83)
DNEA,
(SEQ ID NO: 84)
DNE,
(SEQ ID NO: 85)
NEAYEM,
(SEQ ID NO: 86)
NEAYE,
(SEQ ID NO: 87)
NEAY,
(SEQ ID NO: 88)
NEA,
(SEQ ID NO: 89)
EAYEM,
(SEQ ID NO: 90)
EAYE,
(SEQ ID NO: 91)
EAY,
(SEQ ID NO: 92)
AYEM,
(SEQ ID NO: 93)
AYE,
(SEQ ID NO: 94)
YEM,
(SEQ ID NO: 95)
ATGFVKKDQL,
(SEQ ID NO: 96)
ATGFVKKDQ,
(SEQ ID NO: 97)
ATGFVKKD,
(SEQ ID NO: 98)
ATGFVKK,
(SEQ ID NO: 99)
ATGFVK,
(SEQ ID NO: 100)
ATGFV,
(SEQ ID NO: 101)
ATGF,
(SEQ ID NO: 102)
ATG,
(SEQ ID NO: 103)
TGFVKKDQL,
(SEQ ID NO: 104)
TGFVKKDQ,
(SEQ ID NO: 105)
TGFVKKD,
(SEQ ID NO: 106)
TGFVKK,
(SEQ ID NO: 107)
TGFVK,
(SEQ ID NO: 108)
TGFV,
(SEQ ID NO: 109)
TGF,
(SEQ ID NO: 110)
GFVKKDQL,
(SEQ ID NO: 111)
GFVKKDQ,
(SEQ ID NO: 112)
GFVKKD,
(SEQ ID NO: 113)
GFVKK,
(SEQ ID NO: 114)
GFVK,
(SEQ ID NO: 115)
GFV,
(SEQ ID NO: 116)
FVKKDQL,
(SEQ ID NO: 117)
FVKKDQ,
(SEQ ID NO: 118)
FVKKD,
(SEQ ID NO: 119)
FVKK,
(SEQ ID NO: 120)
FVK,
(SEQ ID NO: 121)
VKKDQL,
(SEQ ID NO: 122)
VKKDQ,
(SEQ ID NO: 123)
VKKD,
(SEQ ID NO: 124)
VKK,
(SEQ ID NO: 125)
KKDQL,
(SEQ ID NO: 126)
KKDQ,
(SEQ ID NO: 127)
KKD,
(SEQ ID NO: 128)
KDQL,
(SEQ ID NO: 129)
KDQ,
and
(d) optionally comprising a fourth polypeptide selected from an amino acid sequence of SEQ ID NO: 3-38, 1002-1057, 39-57, 142-1000, and 59-129;
wherein each of the first peptide sequence, the second peptide sequence, the third peptide sequence, and the fourth peptide sequence, if present, may optionally comprise a C-terminal cysteine.

41. The immunotherapy composition of claim 40, wherein the at least one of the first peptide sequence, the second peptide sequence, the third peptide sequence, and the fourth peptide sequence, if present, further comprise a linker to a carrier at either a C-terminal portion of the polypeptide, or a N-terminal portion of the polypeptide.

42. The immunotherapy composition of claim 41, wherein the linker comprises an amino acid sequence selected from the group consisting of GG, GGG, AA, AAA, KK, KKK, SS, SSS, GAGA (SEQ ID NO:139), AGAG (SEQ ID NO:140), and KGKG (SEQ ID NO:141).

43. The immunotherapy composition of claim 42, wherein the linker to the carrier may optionally comprise a C-terminal cysteine (C).

44. The immunotherapy composition of claim 42, wherein the carrier comprises serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

45. The immunotherapy composition of claim 44, wherein the carrier is CRM197.

46. The immunotherapy composition of claim 44, wherein the carrier is diphtheria toxoid.

47. The immunotherapy composition of claim 38, further comprising at least one pharmaceutically acceptable diluent.

48. The immunotherapy composition of claim 38, further comprising a multiple antigen presenting system (MAP).

49. The immunotherapy composition of claim 48, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

50. A pharmaceutical composition comprising the immunotherapy composition of claim 38 and an adjuvant.

51. The pharmaceutical composition of claim 50, wherein the adjuvant is selected from the group consisting of aluminum hydroxide, aluminum phosphate, aluminum sulfate, 3 De-O-acylated monophosphoryl lipid A (MPL), QS-21, TQL1055, QS-18, QS-17, QS-7, Complete Freund's Adjuvant (CFA), Incomplete Freund's Adjuvant (IFA), oil in water emulsions (such as squalene or peanut oil), CpG, polyglutamic acid, polylysine, AddaVax™, MF59®, and combinations thereof.

52. The pharmaceutical composition of claim 51, wherein the adjuvant is QS-21 or TQL1055.

53. The pharmaceutical composition of claim 51, wherein the adjuvant is MPL.

54. The pharmaceutical composition of claim 51, wherein the adjuvant is a combination of MPL and QS-21 or a combination of MPL and TQL1055.

55. A nucleic acid comprising a nucleic acid sequence encoding a polypeptide of claim 1.

56. A nucleic acid immunotherapy composition comprising the nucleic acid of claim 55 and at least one adjuvant.

57. A method of treating or effecting prophylaxis of Alzheimer's disease in a subject, comprising administrating to the subject the immunotherapy composition of claim 25.

58. A method of inhibiting or reducing aggregation of at least one of A3, tau, and alpha-synuclein in a subject having or at risk of developing Alzheimer's disease, comprising, administering to the subject the immunotherapy composition of claim 25.

59. A method of treating or effecting prophylaxis of Alzheimer's disease in a subject, comprising administrating to the subject the nucleic acid immunotherapy composition of claim 56.

60. A method of inhibiting or reducing aggregation of at least one of Aβ, tau, and alpha-synuclein in a subject having or at risk of developing Alzheimer's disease, comprising administering to the subject the nucleic acid immunotherapy composition of claim 56.

61. The method of claim 57, further comprising repeating the administering at least a second time, at least a third time, at least a fourth time, at least a fifth time, or at least a sixth time.

62. The method of claim 61, further comprising repeating the administering at an interval of about 21 to about 28 days.

63. A method of inducing an immune response in an animal, comprising administering to the animal the immunotherapy composition of claim 25 in a regimen effective to generate an immune response comprising antibodies that specifically bind to Aβ, tau, and/or alpha-synuclein.

64. The method of claim 63, wherein the immune response comprises antibodies that specifically bind to Aβ, antibodies that specifically bind to tau, and antibodies that specifically bind to alpha-synuclein.

65. The method of claim 63, wherein the inducing the immune response comprises antibodies that specifically bind to the N-terminal region of Aβ, the microtubule region of tau, and/or the C-terminal region of alpha-synuclein.

66. An immunization kit comprising the immunotherapy composition of claim 25.

67. The kit of claim 66, further comprising an adjuvant.

68. The kit of claim 67, wherein the immunotherapy composition is in a first container and the adjuvant is in a second container.

69. A kit comprising the nucleic acid immunotherapy composition of claim 56.

70. The kit of claim 69, further comprising an adjuvant.

71. The kit of claim 70, wherein the nucleic acid is in a first container and the adjuvant is in a second container.


SEQUENCE LISTING (1065 sequences)

SEQ ID NO: 1 - protein, 42 aa, Artificial Sequence [sequence omitted]

SEQ ID NO: 2 - protein, 441 aa, Artificial Sequence
  Synthetic peptide
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 10 aa, Artificial Sequence
  Synthetic peptide
DAEFRHDSGY

SEQ ID NO: 4 - protein, 9 aa, Artificial Sequence
  Synthetic peptide
DAEFRHDSG

SEQ ID NO: 5 - protein, 8 aa, Artificial Sequence
  Synthetic peptide
DAEFRHDS

SEQ ID NO: 6 - protein, 7 aa, Artificial Sequence
  Synthetic peptide
DAEFRHD

SEQ ID NO: 7 - protein, 6 aa, Artificial Sequence
  Synthetic peptide
DAEFRH

SEQ ID NO: 8 - protein, 5 aa, Artificial Sequence
  Synthetic peptide
DAEFR

SEQ ID NO: 9 - protein, 4 aa, Artificial Sequence
  Synthetic peptide
DAEF

SEQ ID NO: 10 - protein, 3 aa, Artificial Sequence
  Synthetic peptide
DAE

SEQ ID NO: 11 - protein, 9 aa, Artificial Sequence
  Synthetic peptide
AEFRHDSGY

SEQ ID NO: 12 - protein, 8 aa, Artificial Sequence
  Synthetic peptide
AEFRHDSG

SEQ ID NO: 13 - protein, 7 aa, Artificial Sequence
  Synthetic peptide
AEFRHDS

SEQ ID NO: 14 - protein, 6 aa, Artificial Sequence
  Synthetic peptide
AEFRHD

SEQ ID NO: 15 - protein, 5 aa, Artificial Sequence
  Synthetic peptide
AEFRH

SEQ ID NO: 16 - protein, 4 aa, Artificial Sequence
  Synthetic peptide
AEFR

SEQ ID NO: 17 - protein, 3 aa, Artificial Sequence
  Synthetic peptide
AEF

SEQ ID NO: 18 - protein, 8 aa, Artificial Sequence
  Synthetic peptide
EFRHDSGY

SEQ ID NO: 19 - protein, 7 aa, Artificial Sequence
  Synthetic peptide
EFRHDSG

SEQ ID NO: 20 - protein, 6 aa, Artificial Sequence
  Synthetic peptide
EFRHDS

SEQ ID NO: 21 - protein, 5 aa, Artificial Sequence
  Synthetic peptide
EFRHD

SEQ ID NO: 22 - protein, 4 aa, Artificial Sequence
  Synthetic peptide
EFRH

SEQ ID NO: 23 - protein, 3 aa, Artificial Sequence
  Synthetic peptide
EFR

SEQ ID NO: 24 - protein, 7 aa, Artificial Sequence
  Synthetic peptide
FRHDSGY

SEQ ID NO: 25 - protein, 6 aa, Artificial Sequence
  Synthetic peptide
FRHDSG

SEQ ID NO: 26 - protein, 5 aa, Artificial Sequence
  Synthetic peptide
FRHDS

SEQ ID NO: 27 - protein, 4 aa, Artificial Sequence
  Synthetic peptide
FRHD

SEQ ID NO: 28 - protein, 3 aa, Artificial Sequence
  Synthetic peptide
FRH

SEQ ID NO: 29 - protein, 6 aa, Artificial Sequence
  Synthetic peptide
RHDSGY

SEQ ID NO: 30 - protein, 5 aa, Artificial Sequence
  Synthetic peptide
RHDSG

SEQ ID NO: 31 - protein, 4 aa, Artificial Sequence
  Synthetic peptide
RHDS

SEQ ID NO: 32 - protein, 3 aa, Artificial Sequence
  Synthetic peptide
RHD

SEQ ID NO: 33 - protein, 5 aa, Artificial Sequence
  Synthetic peptide
HDSGY

SEQ ID NO: 34 - protein, 4 aa, Artificial Sequence
  Synthetic peptide
HDSG

SEQ ID NO: 35 - protein, 3 aa, Artificial Sequence
  Synthetic peptide
HDS

SEQ ID NO: 36 - protein, 4 aa, Artificial Sequence
  Synthetic peptide
DSGY

SEQ ID NO: 37 - protein, 3 aa, Artificial Sequence
  Synthetic peptide
DSG

SEQ ID NO: 38 - protein, 3 aa, Artificial Sequence
  Synthetic peptide
SGY

SEQ ID NO: 39 - protein, 7 aa, Artificial Sequence
  Synthetic peptide
QIVYKPV

SEQ ID NO: 40 - protein, 6 aa, Artificial Sequence
  Synthetic peptide
QIVYKP

SEQ ID NO: 41 - protein, 7 aa, Artificial Sequence
  Synthetic peptide
QIVYKSV

SEQ ID NO: 42 - protein, 7 aa, Artificial Sequence
  Synthetic peptide
EIVYKSV

SEQ ID NO: 43 - protein, 7 aa, Artificial Sequence
  Synthetic peptide
EIVYKSP

SEQ ID NO: 44 - protein, 7 aa, Artificial Sequence
  Synthetic peptide
EIVYKPV

SEQ ID NO: 45 - protein, 7 aa, Artificial Sequence
  Synthetic peptide
IVYKSPV

SEQ ID NO: 46 - protein, 4 aa, Artificial Sequence
  Synthetic peptide
IVYK

SEQ ID NO: 47 - protein, 7 aa, Artificial Sequence
  Synthetic peptide
CNIKHVP

SEQ ID NO: 48 - protein, 6 aa, Artificial Sequence
  Synthetic peptide
NIKHVP

SEQ ID NO: 49 - protein, 6 aa, Artificial Sequence
  Synthetic peptide
HVPGGG

SEQ ID NO: 50 - protein, 5 aa, Artificial Sequence
  Synthetic peptide
HVPGG

SEQ ID NO: 51 - protein, 6 aa, Artificial Sequence
  Synthetic peptide
HKPGGG

SEQ ID NO: 52 - protein, 5 aa, Artificial Sequence
  Synthetic peptide
HKPGG

SEQ ID NO: 53 - protein, 7 aa, Artificial Sequence
  Synthetic peptide
KHVPGGG

SEQ ID NO: 54 - protein, 6 aa, Artificial Sequence
  Synthetic peptide
KHVPGG

SEQ ID NO: 55 - protein, 6 aa, Artificial Sequence
  Synthetic peptide
HQPGGG

SEQ ID NO: 56 - protein, 5 aa, Artificial Sequence
  Synthetic peptide
HQPGG

SEQ ID NO: 57 - protein, 7 aa, Artificial Sequence
  Synthetic peptide
NIKHVPG

SEQ ID NO: 58 - protein, 140 aa, Artificial Sequence
  Synthetic peptide
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 59 - protein, 10 aa, Artificial Sequence
  Synthetic peptide
VDPDNEAYEM

SEQ ID NO: 60 - protein, 9 aa, Artificial Sequence
  Synthetic peptide
VDPDNEAYE

SEQ ID NO: 61 - protein, 8 aa, Artificial Sequence
  Synthetic peptide
VDPDNEAY

SEQ ID NO: 62 - protein, 7 aa, Artificial Sequence
  Synthetic peptide
VDPDNEA

SEQ ID NO: 63 - protein, 6 aa, Artificial Sequence
  Synthetic peptide
VDPDNE

SEQ ID NO: 64 - protein, 5 aa, Artificial Sequence
  Synthetic peptide
VDPDN

SEQ ID NO: 65 - protein, 4 aa, Artificial Sequence
  Synthetic peptide
VDPD

SEQ ID NO: 66 - protein, 3 aa, Artificial Sequence
  Synthetic peptide
VDP

SEQ ID NO: 67 - protein, 9 aa, Artificial Sequence
  Synthetic peptide
DPDNEAYEM

SEQ ID NO: 68 - protein, 8 aa, Artificial Sequence
  Synthetic peptide
DPDNEAYE

SEQ ID NO: 69 - protein, 7 aa, Artificial Sequence
  Synthetic peptide
DPDNEAY

SEQ ID NO: 70 - protein, 6 aa, Artificial Sequence
  Synthetic peptide
  MISC_FEATURE (17)..(18): Xaa Xaa can be GG or AA or KK or SS
DPDNEA

SEQ ID NO: 71 - protein, 5 aa, Artificial Sequence
  Synthetic peptide
DPDNE

SEQ ID NO: 72 - protein, 4 aa, Artificial Sequence
  Synthetic peptide
DPDN

SEQ ID NO: 73 - protein, 3 aa, Artificial Sequence
  Synthetic peptide
DPD

SEQ ID NO: 74 - protein, 8 aa, Artificial Sequence
  Synthetic peptide
PDNEAYEM

SEQ ID NO: 75 - protein, 7 aa, Artificial Sequence
  Synthetic peptide
PDNEAYE

SEQ ID NO: 76 - protein, 6 aa, Artificial Sequence
  Synthetic peptide
PDNEAY

SEQ ID NO: 77 - protein, 5 aa, Artificial Sequence
  Synthetic peptide
PDNEA

SEQ ID NO: 78 - protein, 4 aa, Artificial Sequence
  Synthetic peptide
PDNE

SEQ ID NO: 79 - protein, 3 aa, Artificial Sequence
  Synthetic peptide
  MISC_FEATURE (17)..(18): Xaa Xaa can be GG or AA or KK or SS
PDN

SEQ ID NO: 80 - protein, 7 aa, Artificial Sequence
  Synthetic peptide
DNEAYEM

SEQ ID NO: 81 - protein, 6 aa, Artificial Sequence
  Synthetic peptide
DNEAYE

SEQ ID NO: 82 - protein, 5 aa, Artificial Sequence
  Synthetic peptide
DNEAY

SEQ ID NO: 83 - protein, 4 aa, Artificial Sequence
  Synthetic peptide
DNEA

SEQ ID NO: 84 - protein, 3 aa, Artificial Sequence
  Synthetic peptide
DNE

SEQ ID NO: 85 - protein, 6 aa, Artificial Sequence
  Synthetic peptide
NEAYEM

SEQ ID NO: 86 - protein, 5 aa, Artificial Sequence
  Synthetic peptide
NEAYE

SEQ ID NO: 87 - protein, 4 aa, Artificial Sequence
  Synthetic peptide
NEAY

SEQ ID NO: 88 - protein, 3 aa, Artificial Sequence
  Synthetic peptide
NEA

SEQ ID NO: 89 - protein, 5 aa, Artificial Sequence
  Synthetic peptide
EAYEM

SEQ ID NO: 90 - protein, 4 aa, Artificial Sequence
  Synthetic peptide
EAYE

SEQ ID NO: 91 - protein, 3 aa, Artificial Sequence
  Synthetic peptide
EAY

SEQ ID NO: 92 - protein, 4 aa, Artificial Sequence
  Synthetic peptide
AYEM

SEQ ID NO: 93 - protein, 3 aa, Artificial Sequence
  Synthetic peptide
AYE

SEQ ID NO: 94 - protein, 3 aa, Artificial Sequence
  Synthetic peptide
YEM

SEQ ID NO: 95 - protein, 10 aa, Artificial Sequence
  Synthetic peptide
ATGFVKKDQL

SEQ ID NO: 96 - protein, 9 aa, Artificial Sequence
  Synthetic peptide
ATGFVKKDQ

SEQ ID NO: 97 - protein, 8 aa, Artificial Sequence
  Synthetic peptide
ATGFVKKD

SEQ ID NO: 98 - protein, 7 aa, Artificial Sequence
  Synthetic peptide
ATGFVKK

SEQ ID NO: 99 - protein, 6 aa, Artificial Sequence
  Synthetic peptide
ATGFVK

SEQ ID NO: 100 - protein, 5 aa, Artificial Sequence
  Synthetic peptide
ATGFV

SEQ ID NO: 101 - protein, 4 aa, Artificial Sequence
  Synthetic peptide
ATGF

SEQ ID NO: 102 - protein, 3 aa, Artificial Sequence
  Synthetic peptide
ATG

SEQ ID NO: 103 - protein, 9 aa, Artificial Sequence
  Synthetic peptide
TGFVKKDQL

SEQ ID NO: 104 - protein, 8 aa, Artificial Sequence
  Synthetic peptide
TGFVKKDQ

SEQ ID NO: 105 - protein, 7 aa, Artificial Sequence
  Synthetic peptide
TGFVKKD

SEQ ID NO: 106 - protein, 6 aa, Artificial Sequence
  Synthetic peptide
TGFVKK

SEQ ID NO: 107 - protein, 5 aa, Artificial Sequence
  Synthetic peptide
TGFVK

SEQ ID NO: 108 - protein, 4 aa, Artificial Sequence
  Synthetic peptide
TGFV

SEQ ID NO: 109 - protein, 3 aa, Artificial Sequence
  Synthetic peptide
TGF

SEQ ID NO: 110 - protein, 8 aa, Artificial Sequence
  Synthetic peptide
GFVKKDQL

SEQ ID NO: 111 - protein, 7 aa, Artificial Sequence
  Synthetic peptide
GFVKKDQ

SEQ ID NO: 112 - protein, 6 aa, Artificial Sequence
  Synthetic peptide
GFVKKD

SEQ ID NO: 113 - protein, 5 aa, Artificial Sequence
  Synthetic peptide
GFVKK

SEQ ID NO: 114 - protein, 4 aa, Artificial Sequence
  Synthetic peptide
GFVK

SEQ ID NO: 115 - protein, 3 aa, Artificial Sequence
  Synthetic peptide
GFV

SEQ ID NO: 116 - protein, 7 aa, Artificial Sequence
  Synthetic peptide
FVKKDQL

SEQ ID NO: 117 - protein, 6 aa, Artificial Sequence
  Synthetic peptide
FVKKDQ

SEQ ID NO: 118 - protein, 5 aa, Artificial Sequence
  Synthetic peptide
FVKKD

SEQ ID NO: 119 - protein, 4 aa, Artificial Sequence
  Synthetic peptide
FVKK

SEQ ID NO: 120 - protein, 3 aa, Artificial Sequence
  Synthetic peptide
FVK

SEQ ID NO: 121 - protein, 6 aa, Artificial Sequence
  Synthetic peptide
VKKDQL

SEQ ID NO: 122 - protein, 5 aa, Artificial Sequence
  Synthetic peptide
VKKDQ

SEQ ID NO: 123 - protein, 4 aa, Artificial Sequence
  Synthetic peptide
VKKD

SEQ ID NO: 124 - protein, 3 aa, Artificial Sequence
  Synthetic peptide
VKK

SEQ ID NO: 125 - protein, 5 aa, Artificial Sequence
  Synthetic peptide
KKDQL

SEQ ID NO: 126 - protein, 4 aa, Artificial Sequence
  Synthetic peptide
KKDQ

SEQ ID NO: 127 - protein, 3 aa, Artificial Sequence
  Synthetic peptide
KKD

SEQ ID NO: 128 - protein, 4 aa, Artificial Sequence
  Synthetic peptide
KDQL

SEQ ID NO: 129 - protein, 3 aa, Artificial Sequence
  Synthetic peptide
KDQ

SEQ ID NO: 130 - protein, 28 aa, Artificial Sequence
  Synthetic peptide
DAEFRHDRRPDNEAYERRQIVYKPVKKC

SEQ ID NO: 131 - protein, 28 aa, Artificial Sequence
  Synthetic peptide
DAEFRHDRRQIVYKPVRRPDNEAYEKKC

SEQ ID NO: 132 - protein, 28 aa, Artificial Sequence
  Synthetic peptide
DAEFRHDRRPDNEAYERRNIKHVPGKKC

SEQ ID NO: 133 - protein, 28 aa, Artificial Sequence
  Synthetic peptide
DAEFRHDRRNIKHVPGRRPDNEAYEKKC

SEQ ID NO: 134 - protein, 36 aa, Artificial Sequence
  Synthetic peptide
DAEFRHDRRQIVYKPVRRPDNEAYERRNIKHVPGGC

SEQ ID NO: 135 - protein, 37 aa, Artificial Sequence
  Synthetic peptide
DAEFRHDRRDPDNEAYRRNIKHVPGRRQIVYKPVGGC

SEQ ID NO: 136 - protein, 36 aa, Artificial Sequence
  Synthetic peptide
EFRHDSGRRQIVYKPVRRPDNEAYERRNIKHVPGGC

SEQ ID NO: 137 - protein, 37 aa, Artificial Sequence
  Synthetic peptide
EFRHDSGRRDPDNEAYRRNIKHVPGRRQIVYKPVGGC

SEQ ID NO: 138 - protein, 4 aa, Artificial Sequence
  Synthetic peptide
RVRR

SEQ ID NO: 139 - protein, 4 aa, Artificial Sequence
  Synthetic peptide
GAGA

SEQ ID NO: 140 - protein, 4 aa, Artificial Sequence
  Synthetic peptide
AGAG

SEQ ID NO: 141 - protein, 4 aa, Artificial Sequence
  Synthetic peptide
KGKG

SEQ ID NO: 142 - protein, 9 aa, Artificial Sequence
  Synthetic peptide
AEFRHDSGC

SEQ ID NO: 143 - protein, 8 aa, Artificial Sequence
  Synthetic peptide
DAEFRHDC

SEQ ID NO: 144 - protein, 8 aa, Artificial Sequence
  Synthetic peptide
CPDNEAYE

SEQ ID NO: 145 - protein, 8 aa, Artificial Sequence
  Synthetic peptide
DPDNEAYC

SEQ ID NO: 146 - protein, 6 aa, Artificial Sequence
  Synthetic peptide
VQIINK

SEQ ID NO: 147 - protein, 7 aa, Artificial Sequence
  Synthetic peptide
VQIINKK

SEQ ID NO: 148 - protein, 8 aa, Artificial Sequence
  Synthetic peptide
VQIINKKL

SEQ ID NO: 149 - protein, 5 aa, Artificial Sequence
  Synthetic peptide
QIINK

SEQ ID NO: 150 - protein, 6 aa, Artificial Sequence
  Synthetic peptide
QIINKK

SEQ ID NO: 151 - protein, 7 aa, Artificial Sequence
  Synthetic peptide
QIINKKL

SEQ ID NO: 152 - protein, 9 aa, Artificial Sequence
  Synthetic peptide
EAAGHVTQC

SEQ ID NO: 153 - protein, 10 aa, Artificial Sequence
  Synthetic peptide
EAAGHVTQAR

SEQ ID NO: 154 - protein, 9 aa, Artificial Sequence
  Synthetic peptide
AAGHVTQAC

SEQ ID NO: 155 - protein, 9 aa, Artificial Sequence
  Synthetic peptide
AGHVTQARC

SEQ ID NO: 156 - protein, 8 aa, Artificial Sequence
  Synthetic peptide
AGHVTQAR

SEQ ID NO: 157 - protein, 7 aa, Artificial Sequence
  Synthetic peptide
GYTMHQD

SEQ ID NO: 158 - protein, 8 aa, Artificial Sequence
  Synthetic peptide
QGGYTMHC

SEQ ID NO: 159 - protein, 9 aa, Artificial Sequence
  Synthetic peptide
QGGYTMHQD

SEQ ID NO: 160 - protein, 8 aa, Artificial Sequence
  Synthetic peptide
GGYTMHQC

SEQ ID NO: 161 - protein, 10 aa, Artificial Sequence
  Synthetic peptide
VPGGGSVQIV

SEQ ID NO: 162 - protein, 9 aa, Artificial Sequence
  Synthetic peptide
PGGGSVQIV

SEQ ID NO: 163 - protein, 8 aa, Artificial Sequence
  Synthetic peptide
GGGSVQIV

SEQ ID NO: 164 - protein, 7 aa, Artificial Sequence
  Synthetic peptide
GGSVQIV

SEQ ID NO: 165 - protein, 6 aa, Artificial Sequence
  Synthetic peptide
GSVQIV

SEQ ID NO: 166 - protein, 5 aa, Artificial Sequence
  Synthetic peptide
SVQIV

SEQ ID NO: 167 - protein, 4 aa, Artificial Sequence
  Synthetic peptide
VQIV

SEQ ID NO: 168 - protein, 3 aa, Artificial Sequence
  Synthetic peptide
QIV

SEQ ID NO: 169 - protein, 10 aa, Artificial Sequence
  Synthetic peptide
PGGGSVQIVY

SEQ ID NO: 170 - protein, 9 aa, Artificial Sequence
  Synthetic peptide
GGGSVQIVY

SEQ ID NO: 171 - protein, 8 aa, Artificial Sequence
  Synthetic peptide
GGSVQIVY

SEQ ID NO: 172 - protein, 7 aa, Artificial Sequence
  Synthetic peptide
GSVQIVY

SEQ ID NO: 173 - protein, 6 aa, Artificial Sequence
  Synthetic peptide
SVQIVY

SEQ ID NO: 174 - protein, 5 aa, Artificial Sequence
  Synthetic peptide
VQIVY

SEQ ID NO: 175 - protein, 4 aa, Artificial Sequence
  Synthetic peptide
QIVY

SEQ ID NO: 176 - protein, 3 aa, Artificial Sequence
  Synthetic peptide
IVY

SEQ ID NO: 177 - protein, 10 aa, Artificial Sequence
  Synthetic peptide
GGGSVQIVYK

SEQ ID NO: 178 - protein, 9 aa, Artificial Sequence
  Synthetic peptide
GGSVQIVYK

SEQ ID NO: 179 - protein, 8 aa, Artificial Sequence
  Synthetic peptide
GSVQIVYK

SEQ ID NO: 180 - protein, 7 aa, Artificial Sequence
  Synthetic peptide
SVQIVYK

SEQ ID NO: 181 - protein, 6 aa, Artificial Sequence
  Synthetic peptide
VQIVYK

SEQ ID NO: 182 - protein, 5 aa, Artificial Sequence
  Synthetic peptide
QIVYK

SEQ ID NO: 183 - protein, 3 aa, Artificial Sequence
  Synthetic peptide
VYK

SEQ ID NO: 184 - protein, 10 aa, Artificial Sequence
  Synthetic peptide
GGSVQIVYKP

SEQ ID NO: 185 - protein, 9 aa, Artificial Sequence
  Synthetic peptide
GSVQIVYKP

SEQ ID NO: 186 - protein, 8 aa, Artificial Sequence
  Synthetic peptide
SVQIVYKP

SEQ ID NO: 187 - protein, 7 aa, Artificial Sequence
  Synthetic peptide
VQIVYKP

SEQ ID NO: 188 - protein, 5 aa, Artificial Sequence
  Synthetic peptide
IVYKP

SEQ ID NO: 189 - protein, 4 aa, Artificial Sequence
  Synthetic peptide
VYKP

SEQ ID NO: 190 - protein, 3 aa, Artificial Sequence
  Synthetic peptide
YKP

SEQ ID NO: 191 - protein, 10 aa, Artificial Sequence
  Synthetic peptide
GSVQIVYKPV

SEQ ID NO: 192 - protein, 9 aa, Artificial Sequence
  Synthetic peptide
SVQIVYKPV

SEQ ID NO: 193 - protein, 8 aa, Artificial Sequence
  Synthetic peptide
VQIVYKPV

SEQ ID NO: 194 - protein, 6 aa, Artificial Sequence
  Synthetic peptide
IVYKPV

SEQ ID NO: 195 - protein, 5 aa, Artificial Sequence
  Synthetic peptide
VYKPV

SEQ ID NO: 196 - protein, 4 aa, Artificial Sequence
  Synthetic peptide
YKPV

SEQ ID NO: 197 - protein, 3 aa, Artificial Sequence
  Synthetic peptide
KPV

SEQ ID NO: 198 - protein, 10 aa, Artificial Sequence
  Synthetic peptide
SVQIVYKPVD

SEQ ID NO: 199 - protein, 9 aa, Artificial Sequence
  Synthetic peptide
VQIVYKPVD

SEQ ID NO: 200 - protein, 8 aa, Artificial Sequence
  Synthetic peptide
QIVYKPVD

SEQ ID NO: 201 - protein, 7 aa, Artificial Sequence
  Synthetic peptide
IVYKPVD

SEQ ID NO: 202 - skipped

SEQ ID NO: 203 - protein, 6 aa, Artificial Sequence
  Synthetic peptide
VYKPVD

SEQ ID NO: 204 - protein, 5 aa, Artificial Sequence
  Synthetic peptide
YKPVD

SEQ ID NO: 205 - protein, 4 aa, Artificial Sequence
  Synthetic peptide
KPVD

SEQ ID NO: 206 - protein, 3 aa, Artificial Sequence
  Synthetic peptide
PVD

SEQ ID NO: 207 - protein, 10 aa, Artificial Sequence
  Synthetic peptide
VQIVYKPVDL

SEQ ID NO: 208 - protein, 9 aa, Artificial Sequence
  Synthetic peptide
QIVYKPVDL

SEQ ID NO: 209 - protein, 8 aa, Artificial Sequence
  Synthetic peptide
IVYKPVDL

SEQ ID NO: 210 - protein, 7 aa, Artificial Sequence
  Synthetic peptide
VYKPVDL

SEQ ID NO: 211 - protein, 6 aa, Artificial Sequence
  Synthetic peptide
YKPVDL

SEQ ID NO: 212 - protein, 5 aa, Artificial Sequence
  Synthetic peptide
KPVDL

SEQ ID NO: 213 - protein, 4 aa, Artificial Sequence
  Synthetic peptide
PVDL

SEQ ID NO: 214 - protein, 3 aa, Artificial Sequence
  Synthetic peptide
VDL

SEQ ID NO: 215 - protein, 10 aa, Artificial Sequence
  Synthetic peptide
QIVYKPVDLS

SEQ ID NO: 216 - protein, 9 aa, Artificial Sequence
  Synthetic peptide
IVYKPVDLS

SEQ ID NO: 217 - protein, 8 aa, Artificial Sequence
  Synthetic peptide
VYKPVDLS

SEQ ID NO: 218 - protein, 7 aa, Artificial Sequence
  Synthetic peptide
YKPVDLS

SEQ ID NO: 219 - protein, 6 aa, Artificial Sequence
  Synthetic peptide
KPVDLS

SEQ ID NO: 220 - protein, 5 aa, Artificial Sequence
  Synthetic peptide
PVDLS

SEQ ID NO: 221 - protein, 4 aa, Artificial Sequence
  Synthetic peptide
VDLS

SEQ ID NO: 222 - protein, 3 aa, Artificial Sequence
  Synthetic peptide
DLS

SEQ ID NO: 223 - protein, 10 aa, Artificial Sequence
  Synthetic peptide
IVYKPVDLSK

SEQ ID NO: 224 - protein, 9 aa, Artificial Sequence
  Synthetic peptide
VYKPVDLSK

SEQ ID NO: 225 - protein, 8 aa, Artificial Sequence
  Synthetic peptide
YKPVDLSK

SEQ ID NO: 226 - protein, 7 aa, Artificial Sequence
  Synthetic peptide
KPVDLSK

SEQ ID NO: 227 - protein, 6 aa, Artificial Sequence
  Synthetic peptide
PVDLSK

SEQ ID NO: 228 - protein, 5 aa, Artificial Sequence
  Synthetic peptide
VDLSK

SEQ ID NO: 229 - protein, 4 aa, Artificial Sequence
  Synthetic peptide
DLSK

SEQ ID NO: 230 - protein, 3 aa, Artificial Sequence
  Synthetic peptide
LSK

SEQ ID NO: 231 - protein, 10 aa, Artificial Sequence
  Synthetic peptide
VYKPVDLSKV

SEQ ID NO: 232 - protein, 9 aa, Artificial Sequence
  Synthetic peptide
YKPVDLSKV

SEQ ID NO: 233 - protein, 8 aa, Artificial Sequence
  Synthetic peptide
KPVDLSKV

SEQ ID NO: 234 - protein, 7 aa, Artificial Sequence
  Synthetic peptide
PVDLSKV

SEQ ID NO: 235 - protein, 6 aa, Artificial Sequence
  Synthetic peptide
VDLSKV

SEQ ID NO: 236 - protein, 5 aa, Artificial Sequence
  Synthetic peptide
DLSKV

SEQ ID NO: 237 - protein, 4 aa, Artificial Sequence
  Synthetic peptide
LSKV

SEQ ID NO: 238 - protein, 3 aa, Artificial Sequence
  Synthetic peptide
SKV

SEQ ID NO: 239 - protein, 10 aa, Artificial Sequence
  Synthetic peptide
YKPVDLSKVT

SEQ ID NO: 240 - protein, 9 aa, Artificial Sequence
  Synthetic peptide
KPVDLSKVT

SEQ ID NO: 241 - protein, 8 aa, Artificial Sequence
  Synthetic peptide
PVDLSKVT

SEQ ID NO: 242 - protein, 7 aa, Artificial Sequence
  Synthetic peptide
VDLSKVT

SEQ ID NO: 243 - protein, 10 aa, Artificial Sequence
  Synthetic peptide
AKTDHGAEIV

SEQ ID NO: 244 - protein, 9 aa, Artificial Sequence
  Synthetic peptide
KTDHGAEIV

SEQ ID NO: 245 - protein, 8 aa, Artificial Sequence
  Synthetic peptide
TDHGAEIV

SEQ ID NO: 246 - protein, 7 aa, Artificial Sequence
  Synthetic peptide
DHGAEIV

SEQ ID NO: 247 - protein, 6 aa, Artificial Sequence
  Synthetic peptide
HGAEIV

SEQ ID NO: 248 - protein, 5 aa, Artificial Sequence
  Synthetic peptide
GAEIV

SEQ ID NO: 249 - protein, 4 aa, Artificial Sequence
  Synthetic peptide
AEIV

SEQ ID NO: 250 - protein, 3 aa, Artificial Sequence [sequence omitted]

SEQ ID NO: 251 - protein, 10 aa, Artificial Sequence
  Synthetic peptide
KTDHGAEIVY

SEQ ID NO: 252 - protein, 9 aa, Artificial Sequence
  Synthetic peptide
TDHGAEIVY

SEQ ID NO: 253 - protein, 8 aa, Artificial Sequence
  Synthetic peptide
DHGAEIVY

SEQ ID NO: 254 - protein, 7 aa, Artificial Sequence
  Synthetic peptide
HGAEIVY

SEQ ID NO: 255 - protein, 6 aa, Artificial Sequence
  Synthetic peptide
GAEIVY

SEQ ID NO: 256 - protein, 5 aa, Artificial Sequence
  Synthetic peptide
AEIVY

SEQ ID NO: 257 - protein, 4 aa, Artificial Sequence
  Synthetic peptide
EIVY

SEQ ID NO: 258 - protein, 3 aa, Artificial Sequence
  Synthetic peptide
IVY

SEQ ID NO: 259 - protein, 10 aa, Artificial Sequence
  Synthetic peptide
TDHGAEIVYK

SEQ ID NO: 260 - protein, 9 aa, Artificial Sequence
  Synthetic peptide
DHGAEIVYK

SEQ ID NO: 261 - protein, 8 aa, Artificial Sequence
  Synthetic peptide
HGAEIVYK

SEQ ID NO: 262 - protein, 7 aa, Artificial Sequence
  Synthetic peptide
GAEIVYK

SEQ ID NO: 263 - protein, 6 aa, Artificial Sequence
  Synthetic peptide
AEIVYK

SEQ ID NO: 264 - protein, 5 aa, Artificial Sequence
  Synthetic peptide
EIVYK

SEQ ID NO: 265 - protein, 4 aa, Artificial Sequence
  Synthetic peptide
IVYK

SEQ ID NO: 266 - protein, 10 aa, Artificial Sequence
  Synthetic peptide
DHGAEIVYKS

SEQ ID NO: 267 - protein, 9 aa, Artificial Sequence
  Synthetic peptide
HGAEIVYKS

SEQ ID NO: 268 - protein, 8 aa, Artificial Sequence
  Synthetic peptide
GAEIVYKS

SEQ ID NO: 269 - protein, 7 aa, Artificial Sequence
  Synthetic peptide
AEIVYKS

SEQ ID NO: 270 - protein, 6 aa, Artificial Sequence
  Synthetic peptide
EIVYKS

SEQ ID NO: 271 - protein, 5 aa, Artificial Sequence
  Synthetic peptide
IVYKS

SEQ ID NO: 272 - protein, 4 aa, Artificial Sequence
  Synthetic peptide
VYKS

SEQ ID NO: 273 - protein, 3 aa, Artificial Sequence
  Synthetic peptide
YKS

SEQ ID NO: 274 - protein, 10 aa, Artificial Sequence
  Synthetic peptide
HGAEIVYKSP

SEQ ID NO: 275 - protein, 9 aa, Artificial Sequence
  Synthetic peptide
GAEIVYKSP

SEQ ID NO: 276 - protein, 8 aa, Artificial Sequence
  Synthetic peptide
AEIVYKSP

SEQ ID NO: 277 - protein, 7 aa, Artificial Sequence
  Synthetic peptide
EIVYKSP

SEQ ID NO: 278 - protein, 6 aa, Artificial Sequence
  Synthetic peptide
IVYKSP

SEQ ID NO: 279 - protein, 5 aa, Artificial Sequence
  Synthetic peptide
VYKSP

SEQ ID NO: 280 - protein, 4 aa, Artificial Sequence
  Synthetic peptide
YKSP

SEQ ID NO: 281 - protein, 3 aa, Artificial Sequence
  Synthetic peptide
KSP

SEQ ID NO: 282 - protein, 10 aa, Artificial Sequence
  Synthetic peptide
GAEIVYKSPV

SEQ ID NO: 283 - protein, 9 aa, Artificial Sequence
  Synthetic peptide
AEIVYKSPV

SEQ ID NO: 284 - protein, 8 aa, Artificial Sequence
  Synthetic peptide
EIVYKSPV

SEQ ID NO: 285 - protein, 7 aa, Artificial Sequence
  Synthetic peptide
IVYKSPV

SEQ ID NO: 286 - protein, 6 aa, Artificial Sequence
  Synthetic peptide
VYKSPV

SEQ ID NO: 287 - protein, 5 aa, Artificial Sequence
  Synthetic peptide
YKSPV

SEQ ID NO: 288 - protein, 4 aa, Artificial Sequence
  Synthetic peptide
KSPV

SEQ ID NO: 289 - protein, 3 aa, Artificial Sequence
  Synthetic peptide
SPV

SEQ ID NO: 290 - protein, 10 aa, Artificial Sequence
  Synthetic peptide
AEIVYKSPVV

SEQ ID NO: 291 - protein, 9 aa, Artificial Sequence
  Synthetic peptide
EIVYKSPVV

SEQ ID NO: 292 - protein, 8 aa, Artificial Sequence
  Synthetic peptide
IVYKSPVV

SEQ ID NO: 293 - protein, 7 aa, Artificial Sequence
  Synthetic peptide
VYKSPVV

SEQ ID NO: 294 - protein, 6 aa, Artificial Sequence
  Synthetic peptide
YKSPVV

SEQ ID NO: 295 - protein, 5 aa, Artificial Sequence
  Synthetic peptide
KSPVV

SEQ ID NO: 296 - protein, 4 aa, Artificial Sequence
  Synthetic peptide
SPVV

SEQ ID NO: 297 - protein, 3 aa, Artificial Sequence
  Synthetic peptide
PVV

SEQ ID NO: 298 - protein, 10 aa, Artificial Sequence
  Synthetic peptide
EIVYKSPVVS

SEQ ID NO: 299 - protein, 9 aa, Artificial Sequence
  Synthetic peptide
IVYKSPVVS

SEQ ID NO: 300 - protein, 8 aa, Artificial Sequence
  Synthetic peptide
VYKSPVVS

SEQ ID NO: 301 - protein, 7 aa, Artificial Sequence
  Synthetic peptide
YKSPVVS

SEQ ID NO: 302 - protein, 6 aa, Artificial Sequence
  Synthetic peptide
KSPVVS

SEQ ID NO: 303 - protein, 5 aa, Artificial Sequence
  Synthetic peptide
SPVVS

SEQ ID NO: 304 - protein, 4 aa, Artificial Sequence
  Synthetic peptide
PVVS

SEQ ID NO: 305 - protein, 3 aa, Artificial Sequence
  Synthetic peptide
VVS

SEQ ID NO: 306 - protein, 10 aa, Artificial Sequence
  Synthetic peptide
IVYKSPVVSG

SEQ ID NO: 307 - protein, 9 aa, Artificial Sequence
  Synthetic peptide
VYKSPVVSG

SEQ ID NO: 308 - protein, 8 aa, Artificial Sequence
  Synthetic peptide
YKSPVVSG

SEQ ID NO: 309 - protein, 7 aa, Artificial Sequence
  Synthetic peptide
KSPVVSG

SEQ ID NO: 310 - protein, 6 aa, Artificial Sequence
  Synthetic peptide
SPVVSG

SEQ ID NO: 311 - protein, 5 aa, Artificial Sequence
  Synthetic peptide
PVVSG

SEQ ID NO: 312 - protein, 4 aa, Artificial Sequence
  Synthetic peptide
VVSG

SEQ ID NO: 313 - protein, 3 aa, Artificial Sequence
  Synthetic peptide
VSG

SEQ ID NO: 314 - protein, 10 aa, Artificial Sequence
  Synthetic peptide
VYKSPVVSGD

SEQ ID NO: 315 - protein, 9 aa, Artificial Sequence
  Synthetic peptide
YKSPVVSGD

SEQ ID NO: 316 - protein, 8 aa, Artificial Sequence
  Synthetic peptide
KSPVVSGD

SEQ ID NO: 317 - protein, 7 aa, Artificial Sequence
  Synthetic peptide
SPVVSGD

SEQ ID NO: 318 - protein, 6 aa, Artificial Sequence
  Synthetic peptide
PVVSGD

SEQ ID NO: 319 - protein, 5 aa, Artificial Sequence
  Synthetic peptide
VVSGD

SEQ ID NO: 320 - protein, 4 aa, Artificial Sequence
  Synthetic peptide
VSGD

SEQ ID NO: 321 - protein, 3 aa, Artificial Sequence
  Synthetic peptide
SGD

SEQ ID NO: 322 - protein, 10 aa, Artificial Sequence
  Synthetic peptide
YKSPVVSGDT

SEQ ID NO: 323 - protein, 9 aa, Artificial Sequence
  Synthetic peptide
KSPVVSGDT

SEQ ID NO: 324 - protein, 8 aa, Artificial Sequence
  Synthetic peptide
SPVVSGDT

SEQ ID NO: 325 - protein, 7 aa, Artificial Sequence
  Synthetic peptide
PVVSGDT

SEQ ID NO: 326 - protein, 6 aa, Artificial Sequence
  Synthetic peptide
VVSGDT

SEQ ID NO: 327 - protein, 5 aa, Artificial Sequence
  Synthetic peptide
VSGDT

SEQ ID NO: 328 - protein, 4 aa, Artificial Sequence
  Synthetic peptide
SGDT

SEQ ID NO: 329 - protein, 3 aa, Artificial Sequence
  Synthetic peptide
GDT

SEQ ID NO: 330 - protein, 10 aa, Artificial Sequence
  Synthetic peptide
KSPVVSGDTS

SEQ ID NO: 331 - protein, 9 aa, Artificial Sequence
  Synthetic peptide
SPVVSGDTS

SEQ ID NO: 332 - protein, 8 aa, Artificial Sequence
  Synthetic peptide
PVVSGDTS

SEQ ID NO: 333 - protein, 7 aa, Artificial Sequence
  Synthetic peptide
VVSGDTS

SEQ ID NO: 334 - protein, 6 aa, Artificial Sequence
  Synthetic peptide
VSGDTS

SEQ ID NO: 335 - protein, 5 aa, Artificial Sequence
  Synthetic peptide
SGDTS

SEQ ID NO: 336 - protein, 4 aa, Artificial Sequence
  Synthetic peptide
GDTS

SEQ ID NO: 337 - protein, 3 aa, Artificial Sequence
  Synthetic peptide
DTS

SEQ ID NO: 338 - protein, 10 aa, Artificial Sequence
  Synthetic peptide
SPVVSGDTSP

SEQ ID NO: 339 - protein, 9 aa, Artificial Sequence
  Synthetic peptide
PVVSGDTSP

SEQ ID NO: 340 - protein, 8 aa, Artificial Sequence
  Synthetic peptide
VVSGDTSP

SEQ ID NO: 341 - protein, 7 aa, Artificial Sequence
  Synthetic peptide
VSGDTSP

SEQ ID NO: 342 - protein, 6 aa, Artificial Sequence
  Synthetic peptide
SGDTSP

SEQ ID NO: 343 - protein, 5 aa, Artificial Sequence
  Synthetic peptide
GDTSP

SEQ ID NO: 344 - protein, 4 aa, Artificial Sequence
  Synthetic peptide
DTSP

SEQ ID NO: 345 - protein, 3 aa, Artificial Sequence
  Synthetic peptide
TSP

SEQ ID NO: 346 - protein, 10 aa, Artificial Sequence
  Synthetic peptide
PVVSGDTSPR

SEQ ID NO: 347 - protein, 9 aa, Artificial Sequence
  Synthetic peptide
VVSGDTSPR

SEQ ID NO: 348 - protein, 8 aa, Artificial Sequence
  Synthetic peptide
VSGDTSPR

SEQ ID NO: 349 - protein, 7 aa, Artificial Sequence
  Synthetic peptide
SGDTSPR

SEQ ID NO: 350 - protein, 6 aa, Artificial Sequence
  Synthetic peptide
GDTSPR

SEQ ID NO: 351 - protein, 5 aa, Artificial Sequence
  Synthetic peptide
DTSPR

SEQ ID NO: 352 - protein, 4 aa, Artificial Sequence
  Synthetic peptide
TSPR

SEQ ID NO: 353 - protein, 3 aa, Artificial Sequence
  Synthetic peptide
SPR

SEQ ID NO: 354 - protein, 10 aa, Artificial Sequence
  Synthetic peptide
HQPGGGKVQI

SEQ ID NO: 355 - protein, 9 aa, Artificial Sequence
  Synthetic peptide
QPGGGKVQI

SEQ ID NO: 356 - protein, 8 aa, Artificial Sequence
  Synthetic peptide
PGGGKVQI

SEQ ID NO: 357 - protein, 7 aa, Artificial Sequence
  Synthetic peptide
GGGKVQI

SEQ ID NO: 358 - protein, 6 aa, Artificial Sequence
  Synthetic peptide
GGKVQI

SEQ ID NO: 359 - protein, 5 aa, Artificial Sequence
  Synthetic peptide
GKVQI

SEQ ID NO: 360 - protein, 4 aa, Artificial Sequence
  Synthetic peptide
KVQI

SEQ ID NO: 361 - protein, 3 aa, Artificial Sequence
  Synthetic peptide
VQI

SEQ ID NO: 362 - protein, 10 aa, Artificial Sequence
  Synthetic peptide
QPGGGKVQII

SEQ ID NO: 363 - protein, 9 aa, Artificial Sequence
  Synthetic peptide
PGGGKVQII

SEQ ID NO: 364 - protein, 6 aa, Artificial Sequence
  Synthetic peptide
SVDLSK

SEQ ID NO: 365 - protein, 8 aa, Artificial Sequence
  Synthetic peptide
GGGKVQII

SEQ ID NO: 366 - protein, 7 aa, Artificial Sequence
  Synthetic peptide
GGKVQII

SEQ ID NO: 367 - protein, 6 aa, Artificial Sequence
  Synthetic peptide
GKVQII

SEQ ID NO: 368 - protein, 5 aa, Artificial Sequence
  Synthetic peptide
KVQII

SEQ ID NO: 369 - protein, 4 aa, Artificial Sequence
  Synthetic peptide
VQII

SEQ ID NO: 370 - protein, 3 aa, Artificial Sequence
  Synthetic peptide
QII

SEQ ID NO: 371 - protein, 10 aa, Artificial Sequence
  Synthetic peptide
PGGGKVQIIN

SEQ ID NO: 372 - protein, 9 aa, Artificial Sequence
  Synthetic peptide
GGGKVQIIN

SEQ ID NO: 373 - protein, 8 aa, Artificial Sequence
  Synthetic peptide
GGKVQIIN

SEQ ID NO: 374 - protein, 7 aa, Artificial Sequence
  Synthetic peptide
GKVQIIN

SEQ ID NO: 375 - protein, 6 aa, Artificial Sequence
  Synthetic peptide
KVQIIN

SEQ ID NO: 376 - protein, 5 aa, Artificial Sequence
  Synthetic peptide
VQIIN

SEQ ID NO: 377 - protein, 4 aa, Artificial Sequence
  Synthetic peptide
QIIN

SEQ ID NO: 378 - protein, 3 aa, Artificial Sequence
  Synthetic peptide
IIN

SEQ ID NO: 379 - protein, 10 aa, Artificial Sequence
  Synthetic peptide
GGGKVQIINK

SEQ ID NO: 380 - protein, 9 aa, Artificial Sequence
  Synthetic peptide
GGKVQIINK

SEQ ID NO: 381 - protein, 8 aa, Artificial Sequence
  Synthetic peptide
GKVQIINK

SEQ ID NO: 382 - protein, 7 aa, Artificial Sequence
  Synthetic peptide
KVQIINK

SEQ ID NO: 383 - protein, 4 aa, Artificial Sequence
  Synthetic peptide
IINK

SEQ ID NO: 384 - protein, 3 aa, Artificial Sequence
  Synthetic peptide
INK

SEQ ID NO: 385 - protein, 10 aa, Artificial Sequence
  Synthetic peptide
GGKVQIINKK

SEQ ID NO: 386 - protein, 9 aa, Artificial Sequence
  Synthetic peptide
GKVQIINKK

SEQ ID NO: 387 - protein, 8 aa, Artificial Sequence
  Synthetic peptide
KVQIINKK

SEQ ID NO: 388 - protein, 5 aa, Artificial Sequence
  Synthetic peptide
IINKK

SEQ ID NO: 389 - protein, 4 aa, Artificial Sequence
  Synthetic peptide
INKK

SEQ ID NO: 390 - protein, 3 aa, Artificial Sequence
  Synthetic peptide
NKK

SEQ ID NO: 391 - protein, 10 aa, Artificial Sequence
  Synthetic peptide
GKVQIINKKL

SEQ ID NO: 392 - protein, 9 aa, Artificial Sequence
  Synthetic peptide
KVQIINKKL

SEQ ID NO: 393 - protein, 6 aa, Artificial Sequence
  Synthetic peptide
IINKKL

SEQ ID NO: 394 - protein, 5 aa, Artificial Sequence
  Synthetic peptide
INKKL

SEQ ID NO: 395 - protein, 4 aa, Artificial Sequence
  Synthetic peptide
NKKL

SEQ ID NO: 396 - protein, 3 aa, Artificial Sequence
  Synthetic peptide
KKL

SEQ ID NO: 397 - protein, 10 aa, Artificial Sequence
  Synthetic peptide
KVQIINKKLD

SEQ ID NO: 398 - protein, 9 aa, Artificial Sequence
  Synthetic peptide
VQIINKKLD

SEQ ID NO: 399 - protein, 8 aa, Artificial Sequence
  Synthetic peptide
QIINKKLD

SEQ ID NO: 400 - protein, 7 aa, Artificial Sequence
  Synthetic peptide
IINKKLD

SEQ ID NO: 401 - protein, 6 aa, Artificial Sequence
  Synthetic peptide
INKKLD

SEQ ID NO: 402 - protein, 5 aa, Artificial Sequence
  Synthetic peptide
NKKLD

SEQ ID NO: 403 - protein, 4 aa, Artificial Sequence
  Synthetic peptide
KKLD

SEQ ID NO: 404 - protein, 3 aa, Artificial Sequence
  Synthetic peptide
KLD

SEQ ID NO: 405 - protein, 10 aa, Artificial Sequence
  Synthetic peptide
VQIINKKLDL

SEQ ID NO: 406 - protein, 9 aa, Artificial Sequence
  Synthetic peptide
QIINKKLDL

SEQ ID NO: 407 - protein, 8 aa, Artificial Sequence
  Synthetic peptide
IINKKLDL

SEQ ID NO: 408 - protein, 7 aa, Artificial Sequence
  Synthetic peptide
INKKLDL

SEQ ID NO: 409 - protein, 6 aa, Artificial Sequence
  Synthetic peptide
NKKLDL

SEQ ID NO: 410 - protein, 5 aa, Artificial Sequence
  Synthetic peptide
KKLDL

SEQ ID NO: 411 - protein, 4 aa, Artificial Sequence
  Synthetic peptide
KLDL

SEQ ID NO: 412 - protein, 3 aa, Artificial Sequence
  Synthetic peptide
LDL

SEQ ID NO: 413 - protein, 10 aa, Artificial Sequence
  Synthetic peptide
QIINKKLDLS

SEQ ID NO: 414 - protein, 9 aa, Artificial Sequence
  Synthetic peptide
IINKKLDLS

SEQ ID NO: 415 - protein, 8 aa, Artificial Sequence
  Synthetic peptide
INKKLDLS

SEQ ID NO: 416 - protein, 7 aa, Artificial Sequence
  Synthetic peptide
NKKLDLS

SEQ ID NO: 417 - protein, 6 aa, Artificial Sequence
  Synthetic peptide
KKLDLS

SEQ ID NO: 418 - protein, 5 aa, Artificial Sequence
  Synthetic peptide
KLDLS

SEQ ID NO: 419 - protein, 4 aa, Artificial Sequence
  Synthetic peptide
LDLS

SEQ ID NO: 420 - protein, 10 aa, Artificial Sequence
  Synthetic peptide
IINKKLDLSN

SEQ ID NO: 421 - protein, 9 aa, Artificial Sequence
  Synthetic peptide
INKKLDLSN

SEQ ID NO: 422 - protein, 8 aa, Artificial Sequence
  Synthetic peptide
NKKLDLSN

SEQ ID NO: 423 - protein, 7 aa, Artificial Sequence
  Synthetic peptide
KKLDLSN

SEQ ID NO: 424 - protein, 6 aa, Artificial Sequence
  Synthetic peptide
KLDLSN

SEQ ID NO: 425 - protein, 5 aa, Artificial Sequence
  Synthetic peptide
LDLSN

SEQ ID NO: 426 - protein, 4 aa, Artificial Sequence
  Synthetic peptide
DLSN

SEQ ID NO: 427 - protein, 3 aa, Artificial Sequence
  Synthetic peptide
LSN

SEQ ID NO: 428 - protein, 10 aa, Artificial Sequence
  Synthetic peptide
INKKLDLSNV

SEQ ID NO: 429 - protein, 9 aa, Artificial Sequence
  Synthetic peptide
NKKLDLSNV

SEQ ID NO: 430 - protein, 8 aa, Artificial Sequence
  Synthetic peptide
KKLDLSNV

SEQ ID NO: 431 - protein, 7 aa, Artificial Sequence
  Synthetic peptide
KLDLSNV

SEQ ID NO: 432 - protein, 6 aa, Artificial Sequence
  Synthetic peptide
LDLSNV

SEQ ID NO: 433 - protein, 5 aa, Artificial Sequence
  Synthetic peptide
DLSNV

SEQ ID NO: 434 - protein, 4 aa, Artificial Sequence
  Synthetic peptide
LSNV

SEQ ID NO: 435 - protein, 3 aa, Artificial Sequence
  Synthetic peptide
SNV

SEQ ID NO: 436 - protein, 10 aa, Artificial Sequence
  Synthetic peptide
NKKLDLSNVQ

SEQ ID NO: 437 - protein, 9 aa, Artificial Sequence
  Synthetic peptide
KKLDLSNVQ

SEQ ID NO: 438 - protein, 8 aa, Artificial Sequence
  Synthetic peptide
KLDLSNVQ

SEQ ID NO: 439 - protein, 7 aa, Artificial Sequence
  Synthetic peptide
LDLSNVQ

SEQ ID NO: 440 - protein, 6 aa, Artificial Sequence
  Synthetic peptide
DLSNVQ

SEQ ID NO: 441 - protein, 5 aa, Artificial Sequence
  Synthetic peptide
LSNVQ

SEQ ID NO: 442 - protein, 4 aa, Artificial Sequence
  Synthetic peptide
SNVQ

SEQ ID NO: 443 - protein, 3 aa, Artificial Sequence
  Synthetic peptide
NVQ

SEQ ID NO: 444 - protein, 10 aa, Artificial Sequence
  Synthetic peptide
KKLDLSNVQS

SEQ ID NO: 445 - protein, 9 aa, Artificial Sequence
  Synthetic peptide
KLDLSNVQS

SEQ ID NO: 446 - protein, 8 aa, Artificial Sequence
  Synthetic peptide
LDLSNVQS

SEQ ID NO: 447 - protein, 7 aa, Artificial Sequence
  Synthetic peptide
DLSNVQS

SEQ ID NO: 448 - protein, 6 aa, Artificial Sequence
  Synthetic peptide
LSNVQS

SEQ ID NO: 449 - protein, 5 aa, Artificial Sequence
  Synthetic peptide
SNVQS

SEQ ID NO: 450 - protein, 4 aa, Artificial Sequence
  Synthetic peptide
NVQS

SEQ ID NO: 451 - protein, 3 aa, Artificial Sequence
  Synthetic peptide
VQS

SEQ ID NO: 452 - protein, 10 aa, Artificial Sequence
  Synthetic peptide
SKCGSKDNIK

SEQ ID NO: 453 - protein, 9 aa, Artificial Sequence
  Synthetic peptide
KCGSKDNIK

SEQ ID NO: 454 - protein, 8 aa, Artificial Sequence
  Synthetic peptide
CGSKDNIK

SEQ ID NO: 455 - protein, 7 aa, Artificial Sequence
  Synthetic peptide
GSKDNIK

SEQ ID NO: 456 - protein, 6 aa, Artificial Sequence
  Synthetic peptide
SKDNIK

SEQ ID NO: 457 - protein, 5 aa, Artificial Sequence
  Synthetic peptide
KDNIK

SEQ ID NO: 458 - protein, 4 aa, Artificial Sequence
  Synthetic peptide
DNIK

SEQ ID NO: 459 - protein, 3 aa, Artificial Sequence
  Synthetic peptide
NIK

SEQ ID NO: 460 - protein, 10 aa, Artificial Sequence
  Synthetic peptide
KCGSKDNIKH

SEQ ID NO: 461 - protein, 9 aa, Artificial Sequence
  Synthetic peptide
CGSKDNIKH

SEQ ID NO: 462 - protein, 8 aa, Artificial Sequence
  Synthetic peptide
GSKDNIKH

SEQ ID NO: 463 - protein, 7 aa, Artificial Sequence
  Synthetic peptide
SKDNIKH

SEQ ID NO: 464 - protein, 6 aa, Artificial Sequence
  Synthetic peptide
KDNIKH

SEQ ID NO: 465 - protein, 5 aa, Artificial Sequence
  Synthetic peptide
DNIKH

SEQ ID NO: 466 - protein, 4 aa, Artificial Sequence
  Synthetic peptide
NIKH

SEQ ID NO: 467 - protein, 3 aa, Artificial Sequence
  Synthetic peptide
IKH

SEQ ID NO: 468 - protein, 10 aa, Artificial Sequence
  Synthetic peptide
CGSKDNIKHV

SEQ ID NO: 469 - protein, 9 aa, Artificial Sequence
  Synthetic peptide
GSKDNIKHV

SEQ ID NO: 470 - protein, 8 aa, Artificial Sequence
  Synthetic peptide
SKDNIKHV

SEQ ID NO: 471 - protein, 7 aa, Artificial Sequence
  Synthetic peptide
KDNIKHV

SEQ ID NO: 472 - protein, 6 aa, Artificial Sequence
  Synthetic peptide
DNIKHV

SEQ ID NO: 473 - protein, 5 aa, Artificial Sequence
  Synthetic peptide
NIKHV

SEQ ID NO: 474 - protein, 4 aa, Artificial Sequence
  Synthetic peptide
IKHV

SEQ ID NO: 475 - protein, 3 aa, Artificial Sequence
  Synthetic peptide
KHV

SEQ ID NO: 476 - protein, 10 aa, Artificial Sequence
  Synthetic peptide
GSKDNIKHVP

SEQ ID NO: 477 - protein, 9 aa, Artificial Sequence
  Synthetic peptide
SKDNIKHVP

SEQ ID NO: 478 - protein, 8 aa, Artificial Sequence
  Synthetic peptide
KDNIKHVP

SEQ ID NO: 479 - protein, 7 aa, Artificial Sequence
  Synthetic peptide
DNIKHVP

SEQ ID NO: 480 - protein, 5 aa, Artificial Sequence
  Synthetic peptide
IKHVP

SEQ ID NO: 481 - protein, 4 aa, Artificial Sequence
  Synthetic peptide
KHVP

SEQ ID NO: 482 - protein, 3 aa, Artificial Sequence
  Synthetic peptide
HVP

SEQ ID NO: 483 - protein, 10 aa, Artificial Sequence
  Synthetic peptide
SKDNIKHVPG

SEQ ID NO: 484 - protein, 9 aa, Artificial Sequence
  Synthetic peptide
KDNIKHVPG

SEQ ID NO: 485 - protein, 8 aa, Artificial Sequence
  Synthetic peptide
DNIKHVPG

SEQ ID NO: 486 - protein, 7 aa, Artificial Sequence
  Synthetic peptide
NIKHVPG

SEQ ID NO: 487 - protein, 6 aa, Artificial Sequence
  Synthetic peptide
IKHVPG

SEQ ID NO: 488 - protein, 5 aa, Artificial Sequence
  Synthetic peptide
KHVPG

SEQ ID NO: 489 - protein, 4 aa, Artificial Sequence
  Synthetic peptide
HVPG

SEQ ID NO: 490 - protein, 3 aa, Artificial Sequence
  Synthetic peptide
VPG

SEQ ID NO: 491 - protein, 10 aa, Artificial Sequence
  Synthetic peptide
KDNIKHVPGG

SEQ ID NO: 492 - protein, 9 aa, Artificial Sequence
  Synthetic peptide
DNIKHVPGG

SEQ ID NO: 493 - protein, 8 aa, Artificial Sequence
  Synthetic peptide
NIKHVPGG

SEQ ID NO: 494 - protein, 7 aa, Artificial Sequence
  Synthetic peptide
IKHVPGG

SEQ ID NO: 495 - protein, 6 aa, Artificial Sequence
  Synthetic peptide
KHVPGG

SEQ ID NO: 496 - protein, 4 aa, Artificial Sequence
  Synthetic peptide
VPGG

SEQ ID NO: 497 - protein, 3 aa, Artificial Sequence
  Synthetic peptide
PGG

SEQ ID NO: 498 - protein, 10 aa, Artificial Sequence
  Synthetic peptide
DNIKHVPGGG

SEQ ID NO: 499 - protein, 9 aa, Artificial Sequence
  Synthetic peptide
NIKHVPGGG

SEQ ID NO: 500 - protein, 8 aa, Artificial Sequence
  Synthetic peptide
IKHVPGGG

SEQ ID NO: 501 - protein, 5 aa, Artificial Sequence
  Synthetic peptide
VPGGG

SEQ ID NO: 502 - protein, 4 aa, Artificial Sequence
  Synthetic peptide
PGGG

SEQ ID NO: 503 - protein, 3 aa, Artificial Sequence
  Synthetic peptide
GGG

SEQ ID NO: 504 - protein, 10 aa, Artificial Sequence
  Synthetic peptide
NIKHVPGGGS

SEQ ID NO: 505 - protein, 9 aa, Artificial Sequence
  Synthetic peptide
IKHVPGGGS

SEQ ID NO: 506 - protein, 8 aa, Artificial Sequence
  Synthetic peptide
KHVPGGGS

SEQ ID NO: 507 - protein, 7 aa, Artificial Sequence
  Synthetic peptide
HVPGGGS

SEQ ID NO: 508 - protein, 6 aa, Artificial Sequence
  Synthetic peptide
VPGGGS

SEQ ID NO: 509 - protein, 5 aa, Artificial Sequence
  Synthetic peptide
PGGGS

SEQ ID NO: 510 - protein, 4 aa, Artificial Sequence
  Synthetic peptide
GGGS

SEQ ID NO: 511 - protein, 3 aa, Artificial Sequence
  Synthetic peptide
GGS

SEQ ID NO: 512 - protein, 10 aa, Artificial Sequence
  Synthetic peptide
IKHVPGGGSV

SEQ ID NO: 513 - protein, 9 aa, Artificial Sequence
  Synthetic peptide
KHVPGGGSV

SEQ ID NO: 514 - protein, 8 aa, Artificial Sequence
  Synthetic peptide
HVPGGGSV

SEQ ID NO: 515 - protein, 7 aa, Artificial Sequence
  Synthetic peptide
VPGGGSV

SEQ ID NO: 516 - protein, 6 aa, Artificial Sequence
  Synthetic peptide
PGGGSV

SEQ ID NO: 517 - protein, 5 aa, Artificial Sequence
  Synthetic peptide
GGGSV

SEQ ID NO: 518 - protein, 4 aa, Artificial Sequence
  Synthetic peptide
GGSV

SEQ ID NO: 519 - protein, 3 aa, Artificial Sequence
  Synthetic peptide
GSV

SEQ ID NO: 520 - protein, 10 aa, Artificial Sequence
  Synthetic peptide
KHVPGGGSVQ

SEQ ID NO: 521 - protein, 9 aa, Artificial Sequence
  Synthetic peptide
HVPGGGSVQ

SEQ ID NO: 522 - protein, 8 aa, Artificial Sequence
  Synthetic peptide
VPGGGSVQ

SEQ ID NO: 523 - protein, 7 aa, Artificial Sequence
  Synthetic peptide
PGGGSVQ

SEQ ID NO: 524 - protein, 6 aa, Artificial Sequence
  Synthetic peptide
GGGSVQ

SEQ ID NO: 525 - protein, 5 aa, Artificial Sequence
  Synthetic peptide
GGSVQ

SEQ ID NO: 526 - protein, 4 aa, Artificial Sequence
  Synthetic peptide
GSVQ

SEQ ID NO: 527 - protein, 3 aa, Artificial Sequence
  Synthetic peptide
SVQ

SEQ ID NO: 528 - protein, 10 aa, Artificial Sequence
  Synthetic peptide
HVPGGGSVQI

SEQ ID NO: 529 - protein, 9 aa, Artificial Sequence
  Synthetic peptide
VPGGGSVQI

SEQ ID NO: 530 - protein, 8 aa, Artificial Sequence
  Synthetic peptide
PGGGSVQI

SEQ ID NO: 531 - protein, 7 aa, Artificial Sequence
  Synthetic peptide
GGGSVQI

SEQ ID NO: 532 - protein, 6 aa, Artificial Sequence
  Synthetic peptide
GGSVQI

SEQ ID NO: 533 - protein, 5 aa, Artificial Sequence
  Synthetic peptide
GSVQI

SEQ ID NO: 534 - protein, 4 aa, Artificial Sequence
  Synthetic peptide
SVQI

SEQ ID NO: 535 - protein, 10 aa, Artificial Sequence
  Synthetic peptide
GGSVQIVYKS

SEQ ID NO: 536 - protein, 9 aa, Artificial Sequence
  Synthetic peptide
GSVQIVYKS

SEQ ID NO: 537 - protein, 8 aa, Artificial Sequence
  Synthetic peptide
SVQIVYKS

SEQ ID NO: 538 - protein, 7 aa, Artificial Sequence
  Synthetic peptide
VQIVYKS

SEQ ID NO: 539 - protein, 6 aa, Artificial Sequence
  Synthetic peptide
QIVYKS

SEQ ID NO: 540 - protein, 5 aa, Artificial Sequence
  Synthetic peptide
IVYKS

SEQ ID NO: 541 - protein, 4 aa, Artificial Sequence
  Synthetic peptide
VYKS

SEQ ID NO: 542 - protein, 3 aa, Artificial Sequence
  Synthetic peptide
YKS

SEQ ID NO: 543 - protein, 10 aa, Artificial Sequence
  Synthetic peptide
GSVQIVYKSV

SEQ ID NO: 544 - protein, 9 aa, Artificial Sequence
  Synthetic peptide
SVQIVYKSV

SEQ ID NO: 545 - protein, 8 aa, Artificial Sequence
  Synthetic peptide
VQIVYKSV

SEQ ID NO: 546 - protein, 7 aa, Artificial Sequence
  Synthetic peptide
QIVYKSV

SEQ ID NO: 547 - protein, 6 aa, Artificial Sequence
  Synthetic peptide
IVYKSV

SEQ ID NO: 548 - protein, 5 aa, Artificial Sequence
  Synthetic peptide
VYKSV

SEQ ID NO: 549 - protein, 4 aa, Artificial Sequence
  Synthetic peptide
YKSV

SEQ ID NO: 550 - protein, 10 aa, Artificial Sequence
  Synthetic peptide
SVQIVYKSVD

SEQ ID NO: 551 - protein, 9 aa, Artificial Sequence
  Synthetic peptide
VQIVYKSVD

SEQ ID NO: 552 - protein, 8 aa, Artificial Sequence
  Synthetic peptide
QIVYKSVD

SEQ ID NO: 553 - protein, 7 aa, Artificial Sequence
  Synthetic peptide
IVYKSVD

SEQ ID NO: 554 - protein, 6 aa, Artificial Sequence
  Synthetic peptide
VYKSVD

SEQ ID NO: 555 - protein, 5 aa, Artificial Sequence
  Synthetic peptide
YKSVD

SEQ ID NO: 556 - protein, 4 aa, Artificial Sequence
  Synthetic peptide
KSVD

SEQ ID NO: 557 - protein, 3 aa, Artificial Sequence
  Synthetic peptide
SVD

SEQ ID NO: 558 - protein, 10 aa, Artificial Sequence
  Synthetic peptide
VQIVYKSVDL

SEQ ID NO: 559 - protein, 9 aa, Artificial Sequence
  Synthetic peptide
QIVYKSVDL

SEQ ID NO: 560 - protein, 8 aa, Artificial Sequence
  Synthetic peptide
IVYKSVDL

SEQ ID NO: 561 - protein, 7 aa, Artificial Sequence
  Synthetic peptide
VYKSVDL

SEQ ID NO: 562 - protein, 6 aa, Artificial Sequence
  Synthetic peptide
YKSVDL

SEQ ID NO: 563 - protein, 5 aa, Artificial Sequence
  Synthetic peptide
KSVDL

SEQ ID NO: 564 - protein, 4 aa, Artificial Sequence
  Synthetic peptide
SVDL

SEQ ID NO: 565 - protein, 10 aa, Artificial Sequence
  Synthetic peptide
QIVYKSVDLS

SEQ ID NO: 566 - protein, 9 aa, Artificial Sequence
  Synthetic peptide
IVYKSVDLS

SEQ ID NO: 567 - protein, 8 aa, Artificial Sequence
  Synthetic peptide
VYKSVDLS

SEQ ID NO: 568 - protein, 7 aa, Artificial Sequence
  Synthetic peptide
YKSVDLS

SEQ ID NO: 569 - protein, 6 aa, Artificial Sequence
  Synthetic peptide
KSVDLS

SEQ ID NO: 570 - protein, 5 aa, Artificial Sequence
  Synthetic peptide
SVDLS

SEQ ID NO: 571 - protein, 10 aa, Artificial Sequence
  Synthetic peptide
IVYKSVDLSK

SEQ ID NO: 572 - protein, 9 aa, Artificial Sequence
  Synthetic peptide
VYKSVDLSK

SEQ ID NO: 573 - protein, 8 aa, Artificial Sequence
  Synthetic peptide
YKSVDLSK

SEQ ID NO: 574 - protein, 7 aa, Artificial Sequence
  Synthetic peptide
KSVDLSK

SEQ ID NO: 575 - protein, 10 aa, Artificial Sequence
  Synthetic peptide
VYKSVDLSKV

SEQ ID NO: 576 - protein, 9 aa, Artificial Sequence
  Synthetic peptide
YKSVDLSKV

SEQ ID NO: 577 - protein, 8 aa, Artificial Sequence
  Synthetic peptide
KSVDLSKV

SEQ ID NO: 578 - protein, 7 aa, Artificial Sequence
  Synthetic peptide
SVDLSKV

SEQ ID NO: 579 - protein, 10 aa, Artificial Sequence
  Synthetic peptide
YKSVDLSKVT

SEQ ID NO: 580 - protein, 9 aa, Artificial Sequence
  Synthetic peptide
KSVDLSKVT

SEQ ID NO: 581 - protein, 8 aa, Artificial Sequence
  Synthetic peptide
SVDLSKVT

SEQ ID NO: 582 - protein, 6 aa, Artificial Sequence
  Synthetic peptide
DLSKVT

SEQ ID NO: 583 - protein, 5 aa, Artificial Sequence
  Synthetic peptide
LSKVT

SEQ ID NO: 584 - protein, 4 aa, Artificial Sequence
  Synthetic peptide
SKVT

SEQ ID NO: 585 - protein, 3 aa, Artificial Sequence
  Synthetic peptide
KVT

SEQ ID NO: 586 - protein, 10 aa, Artificial Sequence
  Synthetic peptide
HGAEIVYKSV

SEQ ID NO: 587 - protein, 9 aa, Artificial Sequence
  Synthetic peptide
GAEIVYKSV

SEQ ID NO: 588 - protein, 8 aa, Artificial Sequence
  Synthetic peptide
AEIVYKSV

SEQ ID NO: 589 - protein, 10 aa, Artificial Sequence
  Synthetic peptide
GAEIVYKSVV

SEQ ID NO: 590 - protein, 9 aa, Artificial Sequence
  Synthetic peptide
AEIVYKSVV

SEQ ID NO: 591 - protein, 8 aa, Artificial Sequence
  Synthetic peptide
EIVYKSVV

SEQ ID NO: 592 - protein, 7 aa, Artificial Sequence
  Synthetic peptide
IVYKSVV

SEQ ID NO: 593 - protein, 6 aa, Artificial Sequence
  Synthetic peptide
VYKSVV

SEQ ID NO: 594 - protein, 5 aa, Artificial Sequence
  Synthetic peptide
YKSVV

SEQ ID NO: 595 - protein, 4 aa, Artificial Sequence
  Synthetic peptide
KSVV

SEQ ID NO: 596 - protein, 3 aa, Artificial Sequence
  Synthetic peptide
SVV

SEQ ID NO: 597 - protein, 10 aa, Artificial Sequence
  Synthetic peptide
AEIVYKSVVS

SEQ ID NO: 598 - protein, 9 aa, Artificial Sequence
  Synthetic peptide
EIVYKSVVS

SEQ ID NO: 599 - protein, 8 aa, Artificial Sequence
  Synthetic peptide
IVYKSVVS

SEQ ID NO: 600 - protein, 7 aa, Artificial Sequence
  Synthetic peptide
VYKSVVS

SEQ ID NO: 601 - protein, 6 aa, Artificial Sequence
  Synthetic peptide
YKSVVS

SEQ ID NO: 602 - protein, 5 aa, Artificial Sequence
  Synthetic peptide
KSVVS

SEQ ID NO: 603 - protein, 4 aa, Artificial Sequence
  Synthetic peptide
SVVS

SEQ ID NO: 604 - protein, 10 aa, Artificial Sequence
  Synthetic peptide
EIVYKSVVSG

SEQ ID NO: 605 - protein, 9 aa, Artificial Sequence
  Synthetic peptide
IVYKSVVSG

SEQ ID NO: 606 - protein, 8 aa, Artificial Sequence
  Synthetic peptide
VYKSVVSG

SEQ ID NO: 607 - protein, 7 aa, Artificial Sequence
  Synthetic peptide
YKSVVSG

SEQ ID NO: 608 - protein, 6 aa, Artificial Sequence
  Synthetic peptide
KSVVSG

SEQ ID NO: 609 - protein, 5 aa, Artificial Sequence
  Synthetic peptide
SVVSG

SEQ ID NO: 610 - protein, 4 aa, Artificial Sequence
  Synthetic peptide
VVSG

SEQ ID NO: 611 - protein, 3 aa, Artificial Sequence
  Synthetic peptide
VSG

SEQ ID NO: 612 - protein, 10 aa, Artificial Sequence
  Synthetic peptide
IVYKSVVSGD

SEQ ID NO: 613 - protein, 9 aa, Artificial Sequence
  Synthetic peptide
VYKSVVSGD

SEQ ID NO: 614 - protein, 8 aa, Artificial Sequence
  Synthetic peptide
YKSVVSGD

SEQ ID NO: 615 - protein, 7 aa, Artificial Sequence
  Synthetic peptide
KSVVSGD

SEQ ID NO: 616 - protein, 6 aa, Artificial Sequence
  Synthetic peptide
SVVSGD

SEQ ID NO: 617 - protein, 5 aa, Artificial Sequence
  Synthetic peptide
VVSGD

SEQ ID NO: 618 - protein, 10 aa, Artificial Sequence
  Synthetic peptide
VYKSVVSGDT

SEQ ID NO: 619 - protein, 9 aa, Artificial Sequence
  Synthetic peptide
YKSVVSGDT

SEQ ID NO: 620 - protein, 8 aa, Artificial Sequence
  Synthetic peptide
KSVVSGDT

SEQ ID NO: 621 - protein, 7 aa, Artificial Sequence
  Synthetic peptide
SVVSGDT

SEQ ID NO: 622 - protein, 10 aa, Artificial Sequence
  Synthetic peptide
YKSVVSGDTS

SEQ ID NO: 623 - protein, 9 aa, Artificial Sequence
  Synthetic peptide
KSVVSGDTS

SEQ ID NO: 624 - protein, 8 aa, Artificial Sequence
  Synthetic peptide
SVVSGDTS

SEQ ID NO: 625 - protein, 10 aa, Artificial Sequence
  Synthetic peptide
YKSVVSGDTS

SEQ ID NO: 626 - protein, 9 aa, Artificial Sequence
  Synthetic peptide
KSVVSGDTS

SEQ ID NO: 627 - protein, 8 aa, Artificial Sequence
  Synthetic peptide
SVVSGDTS

SEQ ID NO: 628 - protein, 7 aa, Artificial Sequence
  Synthetic peptide
VVSGDTS

SEQ ID NO: 629 - protein, 10 aa, Artificial Sequence
  Synthetic peptide
KSVVSGDTSP

SEQ ID NO: 630 - protein, 9 aa, Artificial Sequence
  Synthetic peptide
SVVSGDTSP

SEQ ID NO: 631 - protein, 10 aa, Artificial Sequence
  Synthetic peptide
SVVSGDTSPR

SEQ ID NO: 632 - protein, 10 aa, Artificial Sequence
  Synthetic peptide
DHGAEIVYKP

SEQ ID NO: 633 - protein, 9 aa, Artificial Sequence
  Synthetic peptide
HGAEIVYKP

SEQ ID NO: 634 - protein, 8 aa, Artificial Sequence
  Synthetic peptide
GAEIVYKP

SEQ ID NO: 635 - protein, 7 aa, Artificial Sequence
  Synthetic peptide
AEIVYKP

SEQ ID NO: 636 - protein, 10 aa, Artificial Sequence
  Synthetic peptide
HGAEIVYKPV

SEQ ID NO: 637 - protein, 9 aa, Artificial Sequence
  Synthetic peptide
GAEIVYKPV

SEQ ID NO: 638 - protein, 8 aa, Artificial Sequence
  Synthetic peptide
AEIVYKPV

SEQ ID NO: 639 - protein, 10 aa, Artificial Sequence
  Synthetic peptide
GAEIVYKPVV

SEQ ID NO: 640 - protein, 9 aa, Artificial Sequence
  Synthetic peptide
AEIVYKPVV

SEQ ID NO: 641 - protein, 8 aa, Artificial Sequence
  Synthetic peptide
EIVYKPVV

SEQ ID NO: 642 - protein, 7 aa, Artificial Sequence
  Synthetic peptide
IVYKPVV

SEQ ID NO: 643 - protein, 6 aa, Artificial Sequence
  Synthetic peptide
VYKPVV

SEQ ID NO: 644 - protein, 5 aa, Artificial Sequence
  Synthetic peptide
YKPVV

SEQ ID NO: 645 - protein, 4 aa, Artificial Sequence
  Synthetic peptide
KPVV

SEQ ID NO: 646 - protein, 10 aa, Artificial Sequence
  Synthetic peptide
AEIVYKPVVS

SEQ ID NO: 647 - protein, 9 aa, Artificial Sequence
  Synthetic peptide
EIVYKPVVS

SEQ ID NO: 648 - protein, 8 aa, Artificial Sequence
  Synthetic peptide
IVYKPVVS

SEQ ID NO: 649 - protein, 7 aa, Artificial Sequence
  Synthetic peptide
VYKPVVS

SEQ ID NO: 650 - protein, 6 aa, Artificial Sequence
  Synthetic peptide
YKPVVS

SEQ ID NO: 651 - protein, 5 aa, Artificial Sequence
  Synthetic peptide
KPVVS

SEQ ID NO: 652 - protein, 10 aa, Artificial Sequence
  Synthetic peptide
EIVYKPVVSG

SEQ ID NO: 653 - protein, 9 aa, Artificial Sequence
  Synthetic peptide
IVYKPVVSG

SEQ ID NO: 654 - protein, 8 aa, Artificial Sequence
  Synthetic peptide
VYKPVVSG

SEQ ID NO: 655 - protein, 7 aa, Artificial Sequence
  Synthetic peptide
YKPVVSG

SEQ ID NO: 656 - protein, 6 aa, Artificial Sequence
  Synthetic peptide
KPVVSG

SEQ ID NO: 657 - protein, 10 aa, Artificial Sequence
  Synthetic peptide
IVYKPVVSGD

SEQ ID NO: 658 - protein, 9 aa, Artificial Sequence
  Synthetic peptide
VYKPVVSGD

SEQ ID NO: 659 - protein, 8 aa, Artificial Sequence
  Synthetic peptide
YKPVVSGD

SEQ ID NO: 660 - protein, 7 aa, Artificial Sequence
  Synthetic peptide
KPVVSGD

SEQ ID NO: 661 - protein, 10 aa, Artificial Sequence
  Synthetic peptide
VYKPVVSGDT

SEQ ID NO: 662 - protein, 9 aa, Artificial Sequence
  Synthetic peptide
YKPVVSGDT

SEQ ID NO: 663 - protein, 8 aa, Artificial Sequence
  Synthetic peptide
KPVVSGDT

SEQ ID NO: 664 - protein, 10 aa, Artificial Sequence
  Synthetic peptide
YKPVVSGDTS

SEQ ID NO: 665 - protein, 9 aa, Artificial Sequence
  Synthetic peptide
KPVVSGDTS

SEQ ID NO: 666 - protein, 8 aa, Artificial Sequence
  Synthetic peptide
PVVSGDTS

SEQ ID NO: 667 - protein, 7 aa, Artificial Sequence
  Synthetic peptide
VVSGDTS

SEQ ID NO: 668 - protein, 10 aa, Artificial Sequence
  Synthetic peptide
KPVVSGDTSP

SEQ ID NO: 669 - protein, 4 aa, Artificial Sequence
  Synthetic peptide
CNIK

SEQ ID NO: 670 - protein, 5 aa, Artificial Sequence
  Synthetic peptide
CNIKH

SEQ ID NO: 671 - protein, 6 aa, Artificial Sequence
  Synthetic peptide
CNIKHV

SEQ ID NO: 672 - protein, 9 aa, Artificial Sequence
  Synthetic peptide
CNIKHVPGG

SEQ ID NO: 673 - protein, 10 aa, Artificial Sequence
  Synthetic peptide
CNIKHVPGGG

SEQ ID NO: 674 - protein, 11 aa, Artificial Sequence
  Synthetic peptide
CNIKHVPGGGS

SEQ ID NO: 675 - protein, 10 aa, Artificial Sequence
  Synthetic peptide
ENLKHQPGGG

SEQ ID NO: 676 - protein, 9 aa, Artificial Sequence
  Synthetic peptide
NLKHQPGGG

SEQ ID NO: 677 - protein, 8 aa, Artificial Sequence
  Synthetic peptide
LKHQPGGG

SEQ ID NO: 678 - protein, 7 aa, Artificial Sequence
  Synthetic peptide
KHQPGGG

SEQ ID NO: 679 - protein, 6 aa, Artificial Sequence
  Synthetic peptide
HQPGGG

SEQ ID NO: 680 - protein, 5 aa, Artificial Sequence
  Synthetic peptide
QPGGG

SEQ ID NO: 681 - protein, 10 aa, Artificial Sequence
  Synthetic peptide
TENLKHQPGG

SEQ ID NO: 682 - protein, 9 aa, Artificial Sequence
  Synthetic peptide
ENLKHQPGG

SEQ ID NO: 683 - protein, 8 aa, Artificial Sequence
  Synthetic peptide
NLKHQPGG

SEQ ID NO: 684 - protein, 7 aa, Artificial Sequence
  Synthetic peptide
LKHQPGG

SEQ ID NO: 685 - protein, 6 aa, Artificial Sequence
  Synthetic peptide
KHQPGG

SEQ ID NO: 686 - protein, 5 aa, Artificial Sequence
  Synthetic peptide
HQPGG

SEQ ID NO: 687 - protein, 4 aa, Artificial Sequence
  Synthetic peptide
QPGG

SEQ ID NO: 688 - protein, 9 aa, Artificial Sequence
  Synthetic peptide
TENLKHQPG

SEQ ID NO: 689 - protein, 8 aa, Artificial Sequence
  Synthetic peptide
ENLKHQPG

SEQ ID NO: 690 - protein, 7 aa, Artificial Sequence
  Synthetic peptide
NLKHQPG

SEQ ID NO: 691 - protein, 6 aa, Artificial Sequence
  Synthetic peptide
LKHQPG

SEQ ID NO: 692 - protein, 5 aa, Artificial Sequence
  Synthetic peptide
KHQPG

SEQ ID NO: 693 - protein, 4 aa, Artificial Sequence
  Synthetic peptide
HQPG

SEQ ID NO: 694 - protein, 3 aa, Artificial Sequence
  Synthetic peptide
QPG

SEQ ID NO: 695 - protein, 8 aa, Artificial Sequence
  Synthetic peptide
TENLKHQP

SEQ ID NO: 696 - protein, 7 aa, Artificial Sequence
  Synthetic peptide
ENLKHQP

SEQ ID NO: 697 - protein, 6 aa, Artificial Sequence
  Synthetic peptide
NLKHQP

SEQ ID NO: 698 - protein, 5 aa, Artificial Sequence
  Synthetic peptide
LKHQP

SEQ ID NO: 699 - protein, 4 aa, Artificial Sequence
  Synthetic peptide
KHQP

SEQ ID NO: 700 - protein, 3 aa, Artificial Sequence
  Synthetic peptide
HQP

SEQ ID NO: 701 - protein, 7 aa, Artificial Sequence
  Synthetic peptide
TENLKHQ

SEQ ID NO: 702 - protein, 6 aa, Artificial Sequence
  Synthetic peptide
ENLKHQ

SEQ ID NO: 703 - protein, 5 aa, Artificial Sequence
  Synthetic peptide
NLKHQ

SEQ ID NO: 704 - protein, 4 aa, Artificial Sequence
  Synthetic peptide
LKHQ

SEQ ID NO: 705 - protein, 3 aa, Artificial Sequence
  Synthetic peptide
KHQ

SEQ ID NO: 706 - protein, 6 aa, Artificial Sequence
  Synthetic peptide
TENLKH

SEQ ID NO: 707 - protein, 5 aa, Artificial Sequence
  Synthetic peptide
ENLKH

SEQ ID NO: 708 - protein, 4 aa, Artificial Sequence
  Synthetic peptide
NLKH

SEQ ID NO: 709 - protein, 3 aa, Artificial Sequence
  Synthetic peptide
LKH

SEQ ID NO: 710 - protein, 5 aa, Artificial Sequence
  Synthetic peptide
TENLK

SEQ ID NO: 711 - protein, 4 aa, Artificial Sequence
  Synthetic peptide
ENLK

SEQ ID NO: 712 - protein, 3 aa, Artificial Sequence
  Synthetic peptide
NLK

SEQ ID NO: 713 - protein, 4 aa, Artificial Sequence
  Synthetic peptide
TENL

SEQ ID NO: 714 - protein, 3 aa, Artificial Sequence
  Synthetic peptide
ENL

SEQ ID NO: 715 - protein, 3 aa, Artificial Sequence
  Synthetic peptide
TEN

SEQ ID NO: 716 - protein, 11 aa, Artificial Sequence
  Synthetic peptide
KDNIKHVPGGG

SEQ ID NO: 717 - protein, 4 aa, Artificial Sequence
  Synthetic peptide
KDNI

SEQ ID NO: 718 - protein, 3 aa, Artificial Sequence
  Synthetic peptide
KDN

SEQ ID NO: 719 - protein, 7 aa, Artificial Sequence
  Synthetic peptide
IKHVGGG

SEQ ID NO: 720 - protein, 6 aa, Artificial Sequence
  Synthetic peptide
IKHVGG

SEQ ID NO: 721 - protein, 5 aa, Artificial Sequence
  Synthetic peptide
IKHVG

SEQ ID NO: 722 - protein, 6 aa, Artificial Sequence
  Synthetic peptide
KHVGGG

SEQ ID NO: 723 - protein, 5 aa, Artificial Sequence
  Synthetic peptide
KHVGG

SEQ ID NO: 724 - protein, 4 aa, Artificial Sequence
  Synthetic peptide
KHVG

SEQ ID NO: 725 - protein, 11 aa, Artificial Sequence
  Synthetic peptide
LGNIHHKPGGG

SEQ ID NO: 726 - protein, 10 aa, Artificial Sequence
  Synthetic peptide
GNIHHKPGGG

SEQ ID NO: 727 - protein, 9 aa, Artificial Sequence
  Synthetic peptide
NIHHKPGGG

SEQ ID NO: 728 - protein, 8 aa, Artificial Sequence
  Synthetic peptide
IHHKPGGG

SEQ ID NO: 729 - protein, 7 aa, Artificial Sequence
  Synthetic peptide
HHKPGGG

SEQ ID NO: 730 - protein, 5 aa, Artificial Sequence
  Synthetic peptide
KPGGG

SEQ ID NO: 731 - protein, 10 aa, Artificial Sequence
  Synthetic peptide
LGNIHHKPGG

SEQ ID NO: 732 - protein, 9 aa, Artificial Sequence
  Synthetic peptide
GNIHHKPGG

SEQ ID NO: 733 - protein, 8 aa, Artificial Sequence
  Synthetic peptide
NIHHKPGG

SEQ ID NO: 734 - protein, 7 aa, Artificial Sequence
  Synthetic peptide
IHHKPGG

SEQ ID NO: 735 - protein, 6 aa, Artificial Sequence
  Synthetic peptide
HHKPGG

SEQ ID NO: 736 - protein, 4 aa, Artificial Sequence
  Synthetic peptide
KPGG

SEQ ID NO: 737 - protein, 9 aa, Artificial Sequence
  Synthetic peptide
LGNIHHKPG

SEQ ID NO: 738 - protein, 8 aa, Artificial Sequence
  Synthetic peptide
GNIHHKPG

SEQ ID NO: 739 - protein, 7 aa, Artificial Sequence
  Synthetic peptide
NIHHKPG

SEQ ID NO: 740 - protein, 6 aa, Artificial Sequence
  Synthetic peptide
IHHKPG

SEQ ID NO: 741 - protein, 5 aa, Artificial Sequence
  Synthetic peptide
HHKPG

SEQ ID NO: 742 - protein, 4 aa, Artificial Sequence
  Synthetic peptide
HKPG

SEQ ID NO: 743 - protein, 3 aa, Artificial Sequence
  Synthetic peptide
KPG

SEQ ID NO: 744 - protein, 8 aa, Artificial Sequence
  Synthetic peptide
LGNIHHKP

SEQ ID NO: 745 - protein, 7 aa, Artificial Sequence
  Synthetic peptide
GNIHHKP

SEQ ID NO: 746 - protein, 6 aa, Artificial Sequence
  Synthetic peptide
NIHHKP

SEQ ID NO: 747 - protein, 5 aa, Artificial Sequence
  Synthetic peptide
IHHKP

SEQ ID NO: 748 - protein, 4 aa, Artificial Sequence
  Synthetic peptide
HHKP

SEQ ID NO: 749 - protein, 3 aa, Artificial Sequence
  Synthetic peptide
HKP

SEQ ID NO: 750 - protein, 7 aa, Artificial Sequence
  Synthetic peptide
LGNIHHK

SEQ ID NO: 751 - protein, 6 aa, Artificial Sequence
  Synthetic peptide
GNIHHK

SEQ ID NO: 752 - protein, 5 aa, Artificial Sequence
  Synthetic peptide
NIHHK

SEQ ID NO: 753 - protein, 4 aa, Artificial Sequence
  Synthetic peptide
IHHK

SEQ ID NO: 754 - protein, 3 aa, Artificial Sequence
  Synthetic peptide
HHK

SEQ ID NO: 755 - protein, 6 aa, Artificial Sequence
  Synthetic peptide
LGNIHH

SEQ ID NO: 756 - protein, 5 aa, Artificial Sequence
  Synthetic peptide
GNIHH

SEQ ID NO: 757 - protein, 4 aa, Artificial Sequence
  Synthetic peptide
NIHH

SEQ ID NO: 758 - protein, 3 aa, Artificial Sequence
  Synthetic peptide
IHH

SEQ ID NO: 759 - protein, 5 aa, Artificial Sequence
  Synthetic peptide
LGNIH

SEQ ID NO: 760 - protein, 4 aa, Artificial Sequence
  Synthetic peptide
GNIH

SEQ ID NO: 761 - protein, 3 aa, Artificial Sequence
  Synthetic peptide
NIH

SEQ ID NO: 762 - protein, 4 aa, Artificial Sequence
  Synthetic peptide
LGNI

SEQ ID NO: 763 - protein, 3 aa, Artificial Sequence
  Synthetic peptide
GNI

SEQ ID NO: 764 - protein, 3 aa, Artificial Sequence
  Synthetic peptide
LGN

SEQ ID NO: 765 - protein, 11 aa, Artificial Sequence
  Synthetic peptide
LDNITHVPGGG

SEQ ID NO: 766 - protein, 10 aa, Artificial Sequence
  Synthetic peptide
DNITHVPGGG

SEQ ID NO: 767 - protein, 9 aa, Artificial Sequence
  Synthetic peptide
NITHVPGGG

SEQ ID NO: 768 - protein, 8 aa, Artificial Sequence
  Synthetic peptide
ITHVPGGG

SEQ ID NO: 769 - protein, 7 aa, Artificial Sequence
  Synthetic peptide
THVPGGG

SEQ ID NO: 770 - protein, 10 aa, Artificial Sequence
  Synthetic peptide
LDNITHVPGG

SEQ ID NO: 771 - protein, 9 aa, Artificial Sequence
  Synthetic peptide
DNITHVPGG

SEQ ID NO: 772 - protein, 8 aa, Artificial Sequence
  Synthetic peptide
NITHVPGG

SEQ ID NO: 773 - protein, 7 aa, Artificial Sequence
  Synthetic peptide
ITHVPGG

SEQ ID NO: 774 - protein, 6 aa, Artificial Sequence
  Synthetic peptide
THVPGG

SEQ ID NO: 775 - protein, 9 aa, Artificial Sequence
  Synthetic peptide
LDNITHVPG

SEQ ID NO: 776 - protein, 8 aa, Artificial Sequence
  Synthetic peptide
DNITHVPG

SEQ ID NO: 777 - protein, 7 aa, Artificial Sequence
  Synthetic peptide
NITHVPG

SEQ ID NO: 778 - protein, 6 aa, Artificial Sequence
  Synthetic peptide
ITHVPG

SEQ ID NO: 779 - protein, 5 aa, Artificial Sequence
  Synthetic peptide
THVPG

SEQ ID NO: 780 - protein, 8 aa, Artificial Sequence
  Synthetic peptide
LDNITHVP

SEQ ID NO: 781 - protein, 7 aa, Artificial Sequence
  Synthetic peptide
DNITHVP

SEQ ID NO: 782 - protein, 6 aa, Artificial Sequence
  Synthetic peptide
NITHVP

SEQ ID NO: 783 - protein, 5 aa, Artificial Sequence
  Synthetic peptide
ITHVP

SEQ ID NO: 784 - protein, 4 aa, Artificial Sequence
  Synthetic peptide
THVP

SEQ ID NO: 785 - protein, 7 aa, Artificial Sequence
  Synthetic peptide
LDNITHV

SEQ ID NO: 786 - protein, 6 aa, Artificial Sequence
  Synthetic peptide
DNITHV

SEQ ID NO: 787 - protein, 5 aa, Artificial Sequence
  Synthetic peptide
NITHV

SEQ ID NO: 788 - protein, 4 aa, Artificial Sequence
  Synthetic peptide
ITHV

SEQ ID NO: 789 - protein, 3 aa, Artificial Sequence
  Synthetic peptide
THV

SEQ ID NO: 790 - protein, 6 aa, Artificial Sequence
  Synthetic peptide
LDNITH

SEQ ID NO: 791 - protein, 5 aa, Artificial Sequence
  Synthetic peptide
DNITH

SEQ ID NO: 792 - protein, 4 aa, Artificial Sequence
  Synthetic peptide
NITH

SEQ ID NO: 793 - protein, 3 aa, Artificial Sequence
  Synthetic peptide
ITH

SEQ ID NO: 794 - protein, 5 aa, Artificial Sequence
  Synthetic peptide
LDNIT

SEQ ID NO: 795 - protein, 4 aa, Artificial Sequence
  Synthetic peptide
DNIT

SEQ ID NO: 796 - protein, 3 aa, Artificial Sequence
  Synthetic peptide
NIT

SEQ ID NO: 797 - protein, 4 aa, Artificial Sequence
  Synthetic peptide
LDNI

SEQ ID NO: 798 - protein, 3 aa, Artificial Sequence
  Synthetic peptide
LDN

SEQ ID NO: 799 - protein, 10 aa, Artificial Sequence
  Synthetic peptide
KNVKSKIGST

SEQ ID NO: 800 - protein, 9 aa, Artificial Sequence
  Synthetic peptide
NVKSKIGST

SEQ ID NO: 801 - protein, 8 aa, Artificial Sequence
  Synthetic peptide
VKSKIGST

SEQ ID NO: 802 - protein, 7 aa, Artificial Sequence
  Synthetic peptide
KSKIGST

SEQ ID NO: 803 - protein, 6 aa, Artificial Sequence
  Synthetic peptide
SKIGST

SEQ ID NO: 804 - protein, 5 aa, Artificial Sequence
  Synthetic peptide
KIGST

SEQ ID NO: 805 - protein, 4 aa, Artificial Sequence
  Synthetic peptide
IGST

SEQ ID NO: 806 - protein, 3 aa, Artificial Sequence
  Synthetic peptide
GST

SEQ ID NO: 807 - protein, 10 aa, Artificial Sequence
  Synthetic peptide
NVKSKIGSTE

SEQ ID NO: 808 - protein, 9 aa, Artificial Sequence
  Synthetic peptide
VKSKIGSTE

SEQ ID NO: 809 - protein, 8 aa, Artificial Sequence
  Synthetic peptide
KSKIGSTE

SEQ ID NO: 810 - protein, 7 aa, Artificial Sequence
  Synthetic peptide
SKIGSTE

SEQ ID NO: 811 - protein, 6 aa, Artificial Sequence
  Synthetic peptide
KIGSTE

SEQ ID NO: 812 - protein, 5 aa, Artificial Sequence
  Synthetic peptide
IGSTE

SEQ ID NO: 813 - protein, 4 aa, Artificial Sequence
  Synthetic peptide
GSTE

SEQ ID NO: 814 - protein, 3 aa, Artificial Sequence
  Synthetic peptide
STE

SEQ ID NO: 815 - protein, 10 aa, Artificial Sequence
  Synthetic peptide
VKSKIGSTEN

SEQ ID NO: 816 - protein, 9 aa, Artificial Sequence
  Synthetic peptide
KSKIGSTEN

SEQ ID NO: 817 - protein, 8 aa, Artificial Sequence
  Synthetic peptide
SKIGSTEN

SEQ ID NO: 818 - protein, 7 aa, Artificial Sequence
  Synthetic peptide
KIGSTEN

SEQ ID NO: 819 - protein, 6 aa, Artificial Sequence
  Synthetic peptide
IGSTEN

SEQ ID NO: 820 - protein, 5 aa, Artificial Sequence
  Synthetic peptide
GSTEN

SEQ ID NO: 821 - protein, 4 aa, Artificial Sequence
  Synthetic peptide
STEN

SEQ ID NO: 822 - protein, 10 aa, Artificial Sequence
  Synthetic peptide
KSKIGSTENL

SEQ ID NO: 823 - protein, 9 aa, Artificial Sequence
  Synthetic peptide
SKIGSTENL

SEQ ID NO: 824 - protein, 8 aa, Artificial Sequence
  Synthetic peptide
KIGSTENL

SEQ ID NO: 825 - protein, 7 aa, Artificial Sequence
  Synthetic peptide
IGSTENL

SEQ ID NO: 826 - protein, 6 aa, Artificial Sequence
  Synthetic peptide
GSTENL

SEQ ID NO: 827 - protein, 5 aa, Artificial Sequence
  Synthetic peptide
STENL

SEQ ID NO: 828 - protein, 10 aa, Artificial Sequence
  Synthetic peptide
SKIGSTENLK

SEQ ID NO: 829 - protein, 9 aa, Artificial Sequence
  Synthetic peptide
KIGSTENLK

SEQ ID NO: 830 - protein, 8 aa, Artificial Sequence
  Synthetic peptide
IGSTENLK

SEQ ID NO: 831 - protein, 7 aa, Artificial Sequence
  Synthetic peptide
GSTENLK

SEQ ID NO: 832 - protein, 6 aa, Artificial Sequence
  Synthetic peptide
STENLK

SEQ ID NO: 833 - protein, 10 aa, Artificial Sequence
  Synthetic peptide
KIGSTENLKH

SEQ ID NO: 834 - protein, 9 aa, Artificial Sequence
  Synthetic peptide
IGSTENLKH

SEQ ID NO: 835 - protein, 8 aa, Artificial Sequence
  Synthetic peptide
GSTENLKH

SEQ ID NO: 836 - protein, 7 aa, Artificial Sequence
  Synthetic peptide
STENLKH

SEQ ID NO: 837 - protein, 10 aa, Artificial Sequence
  Synthetic peptide
IGSTENLKHQ

SEQ ID NO: 838 - protein, 9 aa, Artificial Sequence
  Synthetic peptide
GSTENLKHQ

SEQ ID NO: 839 - protein, 8 aa, Artificial Sequence
  Synthetic peptide
STENLKHQ

SEQ ID NO: 840 - protein, 10 aa, Artificial Sequence
  Synthetic peptide
GSTENLKHQP

SEQ ID NO: 841 - protein, 9 aa, Artificial Sequence
  Synthetic peptide
STENLKHQP

SEQ ID NO: 842 - protein, 10 aa, Artificial Sequence
  Synthetic peptide
STENLKHQPG

SEQ ID NO: 843 - protein, 10 aa, Artificial Sequence
  Synthetic peptide
SNVQSKCGSK

SEQ ID NO: 844 - protein, 9 aa, Artificial Sequence
  Synthetic peptide
NVQSKCGSK

SEQ ID NO: 845 - protein, 8 aa, Artificial Sequence
  Synthetic peptide
VQSKCGSK

SEQ ID NO: 846 - protein, 7 aa, Artificial Sequence
  Synthetic peptide
QSKCGSK

SEQ ID NO: 847 - protein, 6 aa, Artificial Sequence
  Synthetic peptide
SKCGSK

SEQ ID NO: 848 - protein, 5 aa, Artificial Sequence
  Synthetic peptide
KCGSK

SEQ ID NO: 849 - protein, 4 aa, Artificial Sequence
  Synthetic peptide
CGSK

SEQ ID NO: 850 - protein, 3 aa, Artificial Sequence
  Synthetic peptide
GSK

SEQ ID NO: 851 - protein, 10 aa, Artificial Sequence
  Synthetic peptide
NVQSKCGSKD

SEQ ID NO: 852 - protein, 9 aa, Artificial Sequence
  Synthetic peptide
VQSKCGSKD

SEQ ID NO: 853 - protein, 8 aa, Artificial Sequence
  Synthetic peptide
QSKCGSKD

SEQ ID NO: 854 - protein, 7 aa, Artificial Sequence
  Synthetic peptide
SKCGSKD

SEQ ID NO: 855 - protein, 6 aa, Artificial Sequence
  Synthetic peptide
KCGSKD

SEQ ID NO: 856 - protein, 5 aa, Artificial Sequence
  Synthetic peptide
CGSKD

SEQ ID NO: 857 - protein, 4 aa, Artificial Sequence
  Synthetic peptide
GSKD

SEQ ID NO: 858 - protein, 3 aa, Artificial Sequence
  Synthetic peptide
SKD

SEQ ID NO: 859 - protein, 10 aa, Artificial Sequence
  Synthetic peptide
VQSKCGSKDN

SEQ ID NO: 860 - protein, 9 aa, Artificial Sequence
  Synthetic peptide
QSKCGSKDN

SEQ ID NO: 861 - protein, 8 aa, Artificial Sequence
  Synthetic peptide
SKCGSKDN

SEQ ID NO: 862 - protein, 7 aa, Artificial Sequence
  Synthetic peptide
KCGSKDN

SEQ ID NO: 863 - protein, 6 aa, Artificial Sequence
  Synthetic peptide
CGSKDN

SEQ ID NO: 864 - protein, 5 aa, Artificial Sequence
  Synthetic peptide
GSKDN

SEQ ID NO: 865 - protein, 4 aa, Artificial Sequence
  Synthetic peptide
SKDN

SEQ ID NO: 866 - protein, 10 aa, Artificial Sequence
  Synthetic peptide
QSKCGSKDNI

SEQ ID NO: 867 - protein, 9 aa, Artificial Sequence
  Synthetic peptide
SKCGSKDNI

SEQ ID NO: 868 - protein, 8 aa, Artificial Sequence
  Synthetic peptide
KCGSKDNI

SEQ ID NO: 869 - protein, 7 aa, Artificial Sequence
  Synthetic peptide
CGSKDNI

SEQ ID NO: 870 - protein, 6 aa, Artificial Sequence
  Synthetic peptide
GSKDNI

SEQ ID NO: 871 - protein, 5 aa, Artificial Sequence
  Synthetic peptide
SKDNI

SEQ ID NO: 872 - protein, 10 aa, Artificial Sequence
  Synthetic peptide
SKVTSKCGSL

SEQ ID NO: 873 - protein, 9 aa, Artificial Sequence
  Synthetic peptide
KVTSKCGSL

SEQ ID NO: 874 - protein, 8 aa, Artificial Sequence
  Synthetic peptide
VTSKCGSL

SEQ ID NO: 875 - protein, 7 aa, Artificial Sequence
  Synthetic peptide
TSKCGSL

SEQ ID NO: 876 - protein, 6 aa, Artificial Sequence
  Synthetic peptide
SKCGSL

SEQ ID NO: 877 - protein, 5 aa, Artificial Sequence
  Synthetic peptide
KCGSL

SEQ ID NO: 878 - protein, 4 aa, Artificial Sequence
  Synthetic peptide
CGSL

SEQ ID NO: 879 - protein, 3 aa, Artificial Sequence
  Synthetic peptide
GSL

SEQ ID NO: 880 - protein, 10 aa, Artificial Sequence
  Synthetic peptide
KVTSKCGSLG

SEQ ID NO: 881 - protein, 9 aa, Artificial Sequence
  Synthetic peptide
VTSKCGSLG

SEQ ID NO: 882 - protein, 8 aa, Artificial Sequence
  Synthetic peptide
TSKCGSLG

SEQ ID NO: 883 - protein, 7 aa, Artificial Sequence
  Synthetic peptide
SKCGSLG

SEQ ID NO: 884 - protein, 6 aa, Artificial Sequence
  Synthetic peptide
KCGSLG

SEQ ID NO: 885 - protein, 5 aa, Artificial Sequence
  Synthetic peptide
CGSLG

SEQ ID NO: 886 - protein, 4 aa, Artificial Sequence
  Synthetic peptide
GSLG

SEQ ID NO: 887 - protein, 3 aa, Artificial Sequence
  Synthetic peptide
SLG

SEQ ID NO: 888 - protein, 10 aa, Artificial Sequence
  Synthetic peptide
VTSKCGSLGN

SEQ ID NO: 889 - protein, 9 aa, Artificial Sequence
  Synthetic peptide
TSKCGSLGN

SEQ ID NO: 890 - protein, 8 aa, Artificial Sequence
  Synthetic peptide
SKCGSLGN

SEQ ID NO: 891 - protein, 7 aa, Artificial Sequence
  Synthetic peptide
KCGSLGN

SEQ ID NO: 892 - protein, 6 aa, Artificial Sequence
  Synthetic peptide
CGSLGN

SEQ ID NO: 893 - protein, 5 aa, Artificial Sequence
  Synthetic peptide
GSLGN

SEQ ID NO: 894 - protein, 4 aa, Artificial Sequence
  Synthetic peptide
SLGN

SEQ ID NO: 895 - protein, 10 aa, Artificial Sequence
  Synthetic peptide
TSKCGSLGNI

SEQ ID NO: 896 - protein, 9 aa, Artificial Sequence
  Synthetic peptide
SKCGSLGNI

SEQ ID NO: 897 - protein, 8 aa, Artificial Sequence
  Synthetic peptide
KCGSLGNI

SEQ ID NO: 898 - protein, 7 aa, Artificial Sequence
  Synthetic peptide
CGSLGNI

SEQ ID NO: 899 - protein, 6 aa, Artificial Sequence
  Synthetic peptide
GSLGNI

SEQ ID NO: 900 - protein, 5 aa, Artificial Sequence
  Synthetic peptide
SLGNI

SEQ ID NO: 901 - protein, 10 aa, Artificial Sequence
  Synthetic peptide
SKCGSLGNIH

SEQ ID NO: 902 - protein, 9 aa, Artificial Sequence
  Synthetic peptide
KCGSLGNIH

SEQ ID NO: 903 - protein, 8 aa, Artificial Sequence
  Synthetic peptide
CGSLGNIH

SEQ ID NO: 904 - protein, 7 aa, Artificial Sequence
  Synthetic peptide
GSLGNIH

SEQ ID NO: 905 - protein, 6 aa, Artificial Sequence
  Synthetic peptide
SLGNIH

SEQ ID NO: 906 - protein, 10 aa, Artificial Sequence
  Synthetic peptide
KCGSLGNIHH

SEQ ID NO: 907 - protein, 9 aa, Artificial Sequence
  Synthetic peptide
CGSLGNIHH

SEQ ID NO: 908 - protein, 8 aa, Artificial Sequence
  Synthetic peptide
GSLGNIHH

SEQ ID NO: 909 - protein, 7 aa, Artificial Sequence
  Synthetic peptide
SLGNIHH

SEQ ID NO: 910 - protein, 10 aa, Artificial Sequence
  Synthetic peptide
CGSLGNIHHK

SEQ ID NO: 911 - protein, 9 aa, Artificial Sequence
  Synthetic peptide
GSLGNIHHK

SEQ ID NO: 912 - protein, 8 aa, Artificial Sequence
  Synthetic peptide
SLGNIHHK

SEQ ID NO: 913 - protein, 10 aa, Artificial Sequence
  Synthetic peptide
GSLGNIHHKP

SEQ ID NO: 914 - protein, 9 aa, Artificial Sequence
  Synthetic peptide
SLGNIHHKP

SEQ ID NO: 915 - protein, 10 aa, Artificial Sequence
  Synthetic peptide
SLGNIHHKPG

SEQ ID NO: 916 - protein, 10 aa, Artificial Sequence
  Synthetic peptide
DRVQSKIGSL

SEQ ID NO: 917 - protein, 9 aa, Artificial Sequence
  Synthetic peptide
RVQSKIGSL

SEQ ID NO: 918 - protein, 8 aa, Artificial Sequence
  Synthetic peptide
VQSKIGSL

SEQ ID NO: 919 - protein, 7 aa, Artificial Sequence
  Synthetic peptide
QSKIGSL

SEQ ID NO: 920 - protein, 6 aa, Artificial Sequence
  Synthetic peptide
SKIGSL

SEQ ID NO: 921 - protein, 5 aa, Artificial Sequence
  Synthetic peptide
KIGSL

SEQ ID NO: 922 - protein, 4 aa, Artificial Sequence
  Synthetic peptide
IGSL

SEQ ID NO: 923 - protein, 10 aa, Artificial Sequence
  Synthetic peptide
RVQSKIGSLD

SEQ ID NO: 924 - protein, 9 aa, Artificial Sequence
  Synthetic peptide
VQSKIGSLD

SEQ ID NO: 925 - protein, 8 aa, Artificial Sequence
  Synthetic peptide
QSKIGSLD

SEQ ID NO: 926 - protein, 7 aa, Artificial Sequence
  Synthetic peptide
SKIGSLD

SEQ ID NO: 927 - protein, 6 aa, Artificial Sequence
  Synthetic peptide
KIGSLD

SEQ ID NO: 928 - protein, 5 aa, Artificial Sequence
  Synthetic peptide
IGSLD

SEQ ID NO: 929 - protein, 4 aa, Artificial Sequence
  Synthetic peptide
GSLD

SEQ ID NO: 930 - protein, 3 aa, Artificial Sequence
  Synthetic peptide
SLD

SEQ ID NO: 931 - protein, 10 aa, Artificial Sequence
  Synthetic peptide
VQSKIGSLDN

SEQ ID NO: 932 - protein, 9 aa, Artificial Sequence
  Synthetic peptide
QSKIGSLDN

SEQ ID NO: 933 - protein, 8 aa, Artificial Sequence
  Synthetic peptide
SKIGSLDN

SEQ ID NO: 934 - protein, 7 aa, Artificial Sequence
  Synthetic peptide
KIGSLDN

SEQ ID NO: 935 - protein, 6 aa, Artificial Sequence
  Synthetic peptide
IGSLDN

SEQ ID NO: 936 - protein, 5 aa, Artificial Sequence
  Synthetic peptide
GSLDN

SEQ ID NO: 937 - protein, 4 aa, Artificial Sequence
  Synthetic peptide
SLDN

SEQ ID NO: 938 - protein, 10 aa, Artificial Sequence
  Synthetic peptide
QSKIGSLDNI

SEQ ID NO: 939 - protein, 9 aa, Artificial Sequence
  Synthetic peptide
SKIGSLDNI

SEQ ID NO: 940 - protein, 8 aa, Artificial Sequence
  Synthetic peptide
KIGSLDNI

SEQ ID NO: 941 - protein, 7 aa, Artificial Sequence
  Synthetic peptide
IGSLDNI

SEQ ID NO: 942 - protein, 6 aa, Artificial Sequence
  Synthetic peptide
GSLDNI

SEQ ID NO: 943 - protein, 10 aa, Artificial Sequence
  Synthetic peptide
SKIGSLDNIT

SEQ ID NO: 944 - protein, 9 aa, Artificial Sequence
  Synthetic peptide
KIGSLDNIT

SEQ ID NO: 945 - protein, 8 aa, Artificial Sequence
  Synthetic peptide
IGSLDNIT

SEQ ID NO: 946 - protein, 7 aa, Artificial Sequence
  Synthetic peptide
GSLDNIT

SEQ ID NO: 947 - protein, 6 aa, Artificial Sequence
  Synthetic peptide
SLDNIT

SEQ ID NO: 948 - protein, 10 aa, Artificial Sequence
  Synthetic peptide
KIGSLDNITH

SEQ ID NO: 949 - protein, 9 aa, Artificial Sequence
  Synthetic peptide
IGSLDNITH

SEQ ID NO: 950 - protein, 8 aa, Artificial Sequence
  Synthetic peptide
GSLDNITH

SEQ ID NO: 951 - protein, 7 aa, Artificial Sequence
  Synthetic peptide
SLDNITH

SEQ ID NO: 952 - protein, 10 aa, Artificial Sequence
  Synthetic peptide
IGSLDNITHV

SEQ ID NO: 953 - protein, 9 aa, Artificial Sequence
  Synthetic peptide
GSLDNITHV

SEQ ID NO: 954 - protein, 8 aa, Artificial Sequence
  Synthetic peptide
SLDNITHV

SEQ ID NO: 955 - protein, 10 aa, Artificial Sequence
  Synthetic peptide
GSLDNITHVP

SEQ ID NO: 956 - protein, 9 aa, Artificial Sequence
  Synthetic peptide
SLDNITHVP

SEQ ID NO: 957 - protein, 10 aa, Artificial Sequence
  Synthetic peptide
SLDNITHVPG

SEQ ID NO: 958 - protein, 8 aa, Artificial Sequence
  Synthetic peptide
PDLKNVKS

SEQ ID NO: 959 - protein, 8 aa, Artificial Sequence
  Synthetic peptide
DLKNVKSK

SEQ ID NO: 960 - protein, 8 aa, Artificial Sequence
  Synthetic peptide
LKNVKSKI

SEQ ID NO: 961 - protein, 8 aa, Artificial Sequence
  Synthetic peptide
KNVKSKIG

SEQ ID NO: 962 - protein, 8 aa, Artificial Sequence
  Synthetic peptide
NVKSKIGS

SEQ ID NO: 963 - protein, 8 aa, Artificial Sequence
  Synthetic peptide
DLSNVQSK

SEQ ID NO: 964 - protein, 8 aa, Artificial Sequence
  Synthetic peptide
LSNVQSKC

SEQ ID NO: 965 - protein, 8 aa, Artificial Sequence
  Synthetic peptide
SNVQSKCG

SEQ ID NO: 966 - protein, 8 aa, Artificial Sequence
  Synthetic peptide
NVQSKCGS

SEQ ID NO: 967 - protein, 8 aa, Artificial Sequence
  Synthetic peptide
VDLSKVTS

SEQ ID NO: 968 - protein, 8 aa, Artificial Sequence
  Synthetic peptide
DLSKVTSK

SEQ ID NO: 969 - protein, 8 aa, Artificial Sequence
  Synthetic peptide
LSKVTSKC

SEQ ID NO: 970 - protein, 8 aa, Artificial Sequence
  Synthetic peptide
SKVTSKCG

SEQ ID NO: 971 - protein, 8 aa, Artificial Sequence
  Synthetic peptide
KVTSKCGS

SEQ ID NO: 972 - protein, 8 aa, Artificial Sequence
  Synthetic peptide
LDFKDRVQ

SEQ ID NO: 973 - protein, 8 aa, Artificial Sequence
  Synthetic peptide
DFKDRVQS

SEQ ID NO: 974 - protein, 8 aa, Artificial Sequence
  Synthetic peptide
FKDRVQSK

SEQ ID NO: 975 - protein, 8 aa, Artificial Sequence
  Synthetic peptide
KDRVQSKI

SEQ ID NO: 976 - protein, 8 aa, Artificial Sequence
  Synthetic peptide
DRVQSKIG

SEQ ID NO: 977 - protein, 8 aa, Artificial Sequence
  Synthetic peptide
RVQSKIGS

SEQ ID NO: 978 - protein, 11 aa, Artificial Sequence
  Synthetic peptide
SKIGSTENLKH

SEQ ID NO: 979 - protein, 11 aa, Artificial Sequence
  Synthetic peptide
SKIGSTENIKH

SEQ ID NO: 980 - protein, 11 aa, Artificial Sequence
  Synthetic peptide
SKIGSKDNLKH

SEQ ID NO: 981 - protein, 11 aa, Artificial Sequence
  Synthetic peptide
SKIGSKENIKH

SEQ ID NO: 982 - protein, 11 aa, Artificial Sequence
  Synthetic peptide
SKIGSLENLKH

SEQ ID NO: 983 - protein, 11 aa, Artificial Sequence
  Synthetic peptide
SKIGSLENIKH

SEQ ID NO: 984 - protein, 11 aa, Artificial Sequence
  Synthetic peptide
SKIGSTDNLKH

SEQ ID NO: 985 - protein, 11 aa, Artificial Sequence
  Synthetic peptide
SKIGSTDNIKH

SEQ ID NO: 986 - protein, 11 aa, Artificial Sequence
  Synthetic peptide
SKIGSKDNIKH

SEQ ID NO: 987 - protein, 11 aa, Artificial Sequence
  Synthetic peptide
SKIGSLDNLKH

SEQ ID NO: 988 - protein, 11 aa, Artificial Sequence
  Synthetic peptide
SKIGSLDNIKH

SEQ ID NO: 989 - protein, 11 aa, Artificial Sequence
  Synthetic peptide
SKIGSTGNLKH

SEQ ID NO: 990 - protein, 11 aa, Artificial Sequence
  Synthetic peptide
SKIGSTGNIKH

SEQ ID NO: 991 - protein, 11 aa, Artificial Sequence
  Synthetic peptide
SKIGSKGNLKH

SEQ ID NO: 992 - protein, 11 aa, Artificial Sequence
  Synthetic peptide
SKIGSKGNIKH

SEQ ID NO: 993 - protein, 11 aa, Artificial Sequence
  Synthetic peptide
SKIGSLGNLKH

SEQ ID NO: 994 - protein, 11 aa, Artificial Sequence
  Synthetic peptide
SKIGSLGNIKH

SEQ ID NO: 995 - protein, 10 aa, Artificial Sequence
  Synthetic peptide
  MISC_FEATURE (2)..(2): Xaa is I or C
  MISC_FEATURE (3)..(3): Xaa is G
  MISC_FEATURE (5)..(5): Xaa is T or K or L
  MISC_FEATURE (6)..(6): Xaa is E or D or G
  MISC_FEATURE (8)..(8): Xaa is L or I
  MISC_FEATURE (9)..(9): Xaa is K or H or T
KXXSXXNXXH

SEQ ID NO: 996 - protein, 6 aa, Artificial Sequence
  Synthetic peptide
  MISC_FEATURE (1)..(1): Xaa can be Q or E
  MISC_FEATURE (6)..(6): Xaa can be S or P
XIVYKX

SEQ ID NO: 997 - protein, 6 aa, Artificial Sequence
  Synthetic peptide
QIVYKS

SEQ ID NO: 998 - protein, 6 aa, Artificial Sequence
  Synthetic peptide
EIVYKS

SEQ ID NO: 999 - protein, 6 aa, Artificial Sequence
  Synthetic peptide
EIVYKP

SEQ ID NO: 1000 - protein, 8 aa, Artificial Sequence
  Synthetic peptide
CNIKHVPG

SEQ ID NO: 1001 - skipped

SEQ ID NO: 1002 - protein, 10 aa, Artificial Sequence
  Synthetic peptide
VHHQKLVFFA

SEQ ID NO: 1003 - protein, 9 aa, Artificial Sequence
  Synthetic peptide
VHHQKLVFF

SEQ ID NO: 1004 - protein, 8 aa, Artificial Sequence
  Synthetic peptide
VHHQKLVF

SEQ ID NO: 1005 - protein, 7 aa, Artificial Sequence
  Synthetic peptide
VHHQKLV

SEQ ID NO: 1006 - protein, 6 aa, Artificial Sequence
  Synthetic peptide
VHHQKL

SEQ ID NO: 1007 - protein, 10 aa, Artificial Sequence
  Synthetic peptide
HHQKLVFFAE

SEQ ID NO: 1008 - protein, 9 aa, Artificial Sequence
  Synthetic peptide
HHQKLVFFA

SEQ ID NO: 1009 - protein, 8 aa, Artificial Sequence
  Synthetic peptide
HHQKLVFF

SEQ ID NO: 1010 - protein, 7 aa, Artificial Sequence
  Synthetic peptide
HHQKLVF

SEQ ID NO: 1011 - protein, 6 aa, Artificial Sequence
  Synthetic peptide
HHQKLV

SEQ ID NO: 1012 - protein, 5 aa, Artificial Sequence
  Synthetic peptide
HHQKL

SEQ ID NO: 1013 - protein, 10 aa, Artificial Sequence
  Synthetic peptide
HQKLVFFAED

SEQ ID NO: 1014 - protein, 9 aa, Artificial Sequence
  Synthetic peptide
HQKLVFFAE

SEQ ID NO: 1015 - protein, 8 aa, Artificial Sequence
  Synthetic peptide
HQKLVFFA

SEQ ID NO: 1016 - protein, 7 aa, Artificial Sequence
  Synthetic peptide
HQKLVFF

SEQ ID NO: 1017 - protein, 6 aa, Artificial Sequence
  Synthetic peptide
HQKLVF

SEQ ID NO: 1018 - protein, 5 aa, Artificial Sequence
  Synthetic peptide
HQKLV

SEQ ID NO: 1019 - protein, 4 aa, Artificial Sequence
  Synthetic peptide
HQKL

SEQ ID NO: 1020 - protein, 10 aa, Artificial Sequence
  Synthetic peptide
QKLVFFAEDV

SEQ ID NO: 1021 - protein, 9 aa, Artificial Sequence
  Synthetic peptide
QKLVFFAED

SEQ ID NO: 1022 - protein, 8 aa, Artificial Sequence
  Synthetic peptide
QKLVFFAE

SEQ ID NO: 1023 - protein, 7 aa, Artificial Sequence
  Synthetic peptide
QKLVFFA

SEQ ID NO: 1024 - protein, 6 aa, Artificial Sequence
  Synthetic peptide
QKLVFF

SEQ ID NO: 1025 - protein, 5 aa, Artificial Sequence
  Synthetic peptide
QKLVF

SEQ ID NO: 1026 - protein, 4 aa, Artificial Sequence
  Synthetic peptide
QKLV

SEQ ID NO: 1027 - protein, 3 aa, Artificial Sequence
  Synthetic peptide
QKL

SEQ ID NO: 1028 - protein, 10 aa, Artificial Sequence
  Synthetic peptide
KLVFFAEDVG

SEQ ID NO: 1029 - protein, 9 aa, Artificial Sequence
  Synthetic peptide
KLVFFAEDV

SEQ ID NO: 1030 - protein, 8 aa, Artificial Sequence
  Synthetic peptide
KLVFFAED

SEQ ID NO: 1031 - protein, 7 aa, Artificial Sequence
  Synthetic peptide
KLVFFAE

SEQ ID NO: 1032 - protein, 6 aa, Artificial Sequence
  Synthetic peptide
KLVFFA

SEQ ID NO: 1033 - protein, 5 aa, Artificial Sequence
  Synthetic peptide
KLVFF

SEQ ID NO: 1034 - protein, 4 aa, Artificial Sequence
  Synthetic peptide
KLVF

SEQ ID NO: 1035 - protein, 4 aa, Artificial Sequence
  Synthetic peptide
KLVF

SEQ ID NO: 1036 - protein, 9 aa, Artificial Sequence
  Synthetic peptide
LVFFAEDVG

SEQ ID NO: 1037 - protein, 8 aa, Artificial Sequence
  Synthetic peptide
LVFFAEDV

SEQ ID NO: 1038 - protein, 7 aa, Artificial Sequence
  Synthetic peptide
LVFFAED

SEQ ID NO: 1039 - protein, 6 aa, Artificial Sequence
  Synthetic peptide
LVFFAE

SEQ ID NO: 1040 - protein, 5 aa, Artificial Sequence
  Synthetic peptide
LVFFA

SEQ ID NO: 1041 - protein, 4 aa, Artificial Sequence
  Synthetic peptide
LVFF

SEQ ID NO: 1042 - protein, 3 aa, Artificial Sequence
  Synthetic peptide
LVF

SEQ ID NO: 1043 - protein, 8 aa, Artificial Sequence
  Synthetic peptide
VFFAEDVG

SEQ ID NO: 1044 - protein, 7 aa, Artificial Sequence
  Synthetic peptide
VFFAEDV

SEQ ID NO: 1045 - protein, 6 aa, Artificial Sequence
  Synthetic peptide
VFFAED

SEQ ID NO: 1046 - protein, 5 aa, Artificial Sequence
  Synthetic peptide
VFFAE

SEQ ID NO: 1047 - protein, 4 aa, Artificial Sequence
  Synthetic peptide
VFFA

SEQ ID NO: 1048 - protein, 3 aa, Artificial Sequence
  Synthetic peptide
VFF

SEQ ID NO: 1049 - protein, 7 aa, Artificial Sequence
  Synthetic peptide
FFAEDVG

SEQ ID NO: 1050 - protein, 6 aa, Artificial Sequence
  Synthetic peptide
FFAEDV

SEQ ID NO: 1051 - protein, 5 aa, Artificial Sequence
  Synthetic peptide
FFAED

SEQ ID NO: 1052 - protein, 4 aa, Artificial Sequence
  Synthetic peptide
FFAE

SEQ ID NO: 1053 - protein, 3 aa, Artificial Sequence
  Synthetic peptide
FFA

SEQ ID NO: 1054 - protein, 6 aa, Artificial Sequence
  Synthetic peptide
FAEDVG

SEQ ID NO: 1055 - protein, 5 aa, Artificial Sequence
  Synthetic peptide
FAEDV

SEQ ID NO: 1056 - protein, 4 aa, Artificial Sequence
  Synthetic peptide
FAED

SEQ ID NO: 1057 - protein, 3 aa, Artificial Sequence
  Synthetic peptide
FAE

SEQ ID NO: 1058 - protein, 30 aa, Artificial Sequence
  Synthetic peptide
DAEFRHDRRDPDNEAYERRENLKHQPGGGC

SEQ ID NO: 1059 - protein, 30 aa, Artificial Sequence
  Synthetic peptide
DAEFRHDRRENLKHQPGRRDPDNEAYEGGC

SEQ ID NO: 1060 - protein, 29 aa, Artificial Sequence
  Synthetic peptide
DAEFRHDRRPDNEAYERRENLKHQPGGGC

SEQ ID NO: 1061 - protein, 29 aa, Artificial Sequence
  Synthetic peptide
DAEFRHDRRENLKHQPGRRPDNEAYEGGC

SEQ ID NO: 1062 - protein, 33 aa, Artificial Sequence
  linker
DAEFRHDRRSKIGSKDNIKHRRDPDNEAYEGGC

SEQ ID NO: 1063 - protein, 33 aa, Artificial Sequence
  linker
DAEFRHDRRDPDNEAYERRSKIGSKDNIKHGGC

SEQ ID NO: 1064 - protein, 4 aa, Artificial Sequence
  Linker
GGGS

SEQ ID NO: 1065 - protein, 5 aa, Artificial Sequence
  Linker
GGGGS